SEQUENCE LISTING (20 sequences)

SEQ ID NO: 1 - protein, 949 aa, Artificial Sequence
  Description of Artificial Sequence Synthetic polypeptide
  ARMAN 1
MRDSITAPRYSSALAARIKEFNSAFKLGIDLGTKTGGVALVKDNKVLLAKTFLDYHKQTLEERRIHRRNRRSRLARRKRIARLRSWILRQKIYGKQLPDPYKIKKMQLPNGVRKGENWIDLVVSGRDLSPEAFVRAITLIFQKRGQRYEEVAKEIEEMSYKEFSTHIKALTSVTEEEFTALAAEIERRQDVVDTDKEAERYTQLSELLSKVSESKSESKDRAQRKEDLGKVVNAFCSAHRIEDKDKWCKELMKLLDRPVRHARFLNKVLIRCNICDRATPKKSRPDVRELLYFDTVRNFLKAGRVEQNPDVISYYKKIYMDAEVIRVKILNKEKLTDEDKKQKRKLASELNRYKNKEYVTDAQKKMQEQLKTLLFMKLTGRSRYCMAHLKERAAGKDVEEGLHGVVQKRHDRNIAQRNHDLRVINLIESLLFDQNKSLSDAIRKNGLMYVTIEAPEPKTKHAKKGAAVVRDPRKLKEKLFDDQNGVCIYTGLQLDKLEISKYEKDHIFPDSRDGPSIRDNLVLTTKEINSDKGDRTPWEWMHDNPEKWKAFERRVAEFYKKGRINERKRELLLNKGTEYPGDNPTELARGGARVNNFITEFNDRLKTHGVQELQTIFERNKPIVQVVRGEETQRLRRQWNALNQNFIPLKDRAMSFNHAEDAAIAASMPPKFWREQIYRTAWHFGPSGNERPDFALAELAPQWNDFFMTKGGPIIAVLGKTKYSWKHSIIDDTIYKPFSKSAYYVGIYKKPNAITSNAIKVLRPKLLNGEHTMSKNAKYYHQKIGNERFLMKSQKGGSIITVKPHDGPEKVLQISPTYECAVLTKHDGKIIVKFKPIKPLRDMYARGVIKAMDKELETSLSSMSKHAKYKELHTHDIIYLPATKKHVDGYFIITKLSAKHGIKALPESMVKVKYTQIGSENNSEVKLTKPKPEITLDSEDITNIYNFTR

SEQ ID NO: 2 - DNA, 2851 bp, Artificial Sequence
  Description of Artificial Sequence Synthetic polynucleotide
  ARMAN 1
atgagagactctattactgcacctagatacagctccgctcttgccgccagaataaaggagtttaattctgctttcaagttaggaatcgacctaggaacaaaaaccggcggcgtagcactggtaaaagacaacaaagtgctgctcgctaagacattcctcgattaccataaacaaacactggaggaaaggaggatccatagaagaaacagaaggagcaggctagccaggcggaagaggattgctcggctgcgatcatggatactcagacagaagatttatggcaagcagcttcctgacccatacaaaatcaaaaaaatgcagttgcctaatggtgtacgaaaaggggaaaactggattgacctggtagtttctggacgggacctttcaccagaagccttcgtgcgtgcaataactctgatattccaaaagagagggcaaagatatgaagaagtggccaaagagatagaagaaatgagttacaaggaatttagtactcacataaaagccctgacatccgttactgaagaagaatttactgctctggcagcagagatagaacggaggcaggatgtggttgacacagacaaggaggccgaacgctatacccaattgtctgagttgctctccaaggtctcagaaagcaaatctgaatctaaagacagagcgcagcgtaaggaggatctcggaaaggtggtgaacgctttctgcagtgctcatcgtatcgaagacaaggataaatggtgtaaagaacttatgaaattactagacagaccagtcagacacgctaggttccttaacaaagtactgatacgttgcaatatctgcgatagggcaacccctaagaaatccagacctgacgtgagggaactgctatattttgacacagtaagaaacttcttgaaggctggaagagtggagcaaaacccagacgttattagttactataaaaaaatttatatggatgcagaagtaatcagggtcaaaattctgaataaggaaaagctgactgatgaggacaaaaagcaaaagaggaaattagcgagcgaacttaacaggtacaaaaacaaagaatacgtgactgatgcgcagaagaagatgcaagagcaacttaagacattgctgttcatgaagctgacaggcaggtctagatactgcatggctcatcttaaggaaagggcagcaggcaaagatgtagaagaaggacttcatggcgttgtgcagaaaagacacgacaggaacatagcacagcgcaatcacgacttacgtgtgattaatcttattgagagtctgcttttcgaccaaaacaaatcgctctccgatgcaataaggaagaacgggttaatgtatgttactattgaggctccagagccaaagactaagcacgcaaagaaaggcgcagctgtggtaagggatcccagaaagttgaaggagaagttgtttgatgatcaaaacggcgtttgcatatatacgggcttgcagttagacaaattagagataagtaaatacgagaaggaccatatctttccagattcaagggatggaccatctatcagggacaatcttgtactcactacaaaagagataaattcagacaaaggcgataggaccccatgggaatggatgcatgataacccagaaaaatggaaagcgttcgagagaagagtcgcagaattctataagaaaggcagaataaatgagaggaaaagagaactcctattaaacaaaggcactgaataccctggcgataacccgactgagctggcgcggggaggcgcccgtgttaacaactttattactgaatttaatgaccgcctcaaaacgcatggagtccaggaactgcagaccatctttgagcgtaacaaaccaatagtgcaggtagtcaggggtgaagaaacgcagcgtctgcgcagacaatggaatgcactaaaccagaatttcataccactaaaggacagggcaatgtcgttcaaccacgctgaagacgcagccatagcagcaagcatgccaccaaaattctggagggagcagatataccgtactgcgtggcactttggacctagtggaaatgagagaccggactttgctttggcagaattggcgccacaatggaatgacttctttatgactaagggcggtccaataatagcagtgctgggcaaaacgaagtatagttggaagcacagcataattgatgacactatatacaagccattcagcaaaagtgcttactatgttgggatatacaaaaagccgaacgccatcacgtccaatgctataaaagtcttaaggccaaaactcttaaatggcgaacatacaatgtctaagaatgcaaagtattatcatcagaagattggtaatgagcgcttcctcatgaaatctcagaaaggtggatcgataattacagtaaaaccacacgacggaccggaaaaagtgcttcaaatcagccctacatatgaatgcgcagtccttactaagcatgacggtaaaataatagtcaaatttaaaccaataaagccgctacgggacatgtatgcccgcggtgtgattaaagccatggacaaagagcttgaaacaagcctctctagcatgagtaaacacgctaagtacaaggagttacacactcatgatatcatatatctgcctgctacaaagaagcacgtagatggctacttcataataaccaaactaagtgcgaaacatggcataaaagcactccccgaaagcatggttaaagtcaagtatactcaaattgggagtgaaaacaatagtgaagtgaagcttaccaaaccaaaaccagagataactttggatagtgaagatattacaaacatatataatttcacccgctaag

SEQ ID NO: 3 - protein, 967 aa, Artificial Sequence
  Description of Artificial Sequence Synthetic polypeptide
  ARMAN 4
MLGSSRYLRYNLTSFEGKEPFLIMGYYKEYNKELSSKAQKEFNDQISEFNSYYKLGIDLGDKTGIAIVKGNKIILAKTLIDLHSQKLDKRREARRNRRTRLSRKKRLARLRSWVMRQKVGNQRLPDPYKIMHDNKYWSIYNKSNSANKKNWIDLLIHSNSLSADDFVRGLTIIFRKRGYLAFKYLSRLSDKEFEKYIDNLKPPISKYEYDEDLEELSSRVENGEIEEKKFEGLKNKLDKIDKESKDFQVKQREEVKKELEDLVDLFAKSVDNKIDKARWKRELNNLLDKKVRKIRFDNRFILKCKIKGCNKNTPKKEKVRDFELKMVLNNARSDYQISDEDLNSFRNEVINIFQKKENLKKGELKGVTIEDLRKQLNKTFNKAKIKKGIREQIRSIVFEKISGRSKFCKEHLKEFSEKPAPSDRINYGVNSAREQHDFRVLNFIDKKIFKDKLIDPSKLRYITIESPEPETEKLEKGQISEKSFETLKEKLAKETGGIDIYTGEKLKKDFEIEHIFPRARMGPSIRENEVASNLETNKEKADRTPWEWFGQDEKRWSEFEKRVNSLYSKKKISERKREILLNKSNEYPGLNPTELSRIPSTLSDFVESIRKMFVKYGYEEPQTLVQKGKPIIQVVRGRDTQALRWRWHALDSNIIPEKDRKSSFNHAEDAVIAACMPPYYLRQKIFREEAKIKRKVSNKEKEVTRPDMPTKKIAPNWSEFMKTRNEPVIEVIGKVKPSWKNSIMDQTFYKYLLKPFKDNLIKIPNVKNTYKWIGVNGQTDSLSLPSKVLSISNKKVDSSTVLLVHDKKGGKRNWVPKSIGGLLVYITPKDGPKRIVQVKPATQGLLIYRNEDGRVDAVREFINPVIEMYNNGKLAFVEKENEEELLKYFNLLEKGQKFERIRRYDMITYNSKFYYVTKINKNHRVTIQEESKIKAESDKVKSSSGKEYTRKETEELSLQKLAELISI

SEQ ID NO: 4 - DNA, 2906 bp, Artificial Sequence
  Description of Artificial Sequence Synthetic polynucleotide
  ARMAN 4
atgttaggctccagcaggtacctccgttataacctaacctcgtttgaaggcaaggagccatttttaataatgggatattacaaagagtataataaggaattaagttccaaagctcaaaaagaatttaatgatcaaatttctgaatttaattcgtattacaaactaggtatagatctcggagataaaacaggaattgcaatcgtaaagggcaacaaaataatcctagcaaaaacactaattgatttgcattcccaaaaattagataaaagaagggaagctagaagaaatagaagaactcggctttccagaaagaaaaggcttgcgagattaagatcgtgggtaatgcgtcagaaagttggcaatcaaagacttcccgatccatataaaataatgcatgacaataagtactggtctatatataataagagtaattctgcaaataaaaagaattggatagatctgttaatccacagtaactctttatcagcagacgattttgttagaggcttaactataattttcagaaaaagaggctatttagcatttaagtatctttcaaggttaagcgataaggaatttgaaaaatacatagataacttaaaaccacctataagcaaatacgagtatgatgaggatttagaagaattatcaagcagggttgaaaatggggaaatagaggaaaagaaattcgaaggcttaaagaataagctagataaaatagacaaagaatctaaagactttcaagtaaagcaaagagaagaagtaaaaaaggaactggaagacttagttgatttgtttgctaaatcagttgataataaaatagataaagctaggtggaaaagggagctaaataatttattggataagaaagtaaggaaaatacggtttgacaaccgctttattttgaagtgcaaaattaagggctgtaacaagaatactccaaagaaagagaaggtcagagattttgaattgaagatggttttaaataatgctagaagcgattatcagatttctgatgaggatttaaactcttttagaaatgaagtaataaatatatttcaaaagaaggaaaacttaaagaaaggagagctgaaaggagttactattgaagatttgagaaagcagcttaataaaacttttaataaagccaagattaaaaaagggataagggagcagataaggtctatcgtgtttgaaaaaattagtggaaggagtaaattctgcaaagaacatctaaaagaattttctgagaagccggctccttctgacaggattaattatggggttaattcagcaagagaacaacatgattttagagtcttaaatttcatagataaaaaaatattcaaagataagttgatagatccctcaaaattgaggtatataactattgaatctccagaaccagaaacagagaagttggaaaaaggtcaaatatcagagaagagcttcgaaacattgaaagaaaaattggctaaagaaacaggtggtattgatatatacactggtgaaaaattaaagaaagactttgaaatagagcacatattcccaagagcaaggatggggccttctataagggaaaacgaagtagcatcaaatctggaaacaaataaggaaaaggccgatagaactccttgggaatggtttgggcaagatgaaaaaagatggtcagagtttgagaaaagagttaattctctttatagtaaaaagaaaatatcagagagaaaaagagaaattttgttaaataagagtaatgaatatccgggattaaaccctacagaactaagtagaatacctagtacgctgagcgacttcgttgagagtataagaaaaatgtttgttaagtatggctatgaagagcctcaaactttggttcaaaaaggaaaaccgataatacaagttgttagaggcagagacacacaagctttgaggtggagatggcatgcattagatagtaatataataccagaaaaggacaggaaaagttcatttaatcacgctgaagatgcagttattgccgcctgtatgccaccttactatctcaggcaaaaaatatttagagaagaagcaaaaataaaaagaaaagtaagcaataaggaaaaggaagttacacggcctgacatgcctactaaaaagatagctccgaactggtcggaatttatgaaaactagaaatgagccggttattgaagtaataggaaaagttaagccaagctggaaaaacagcataatggatcaaacattttataaatatcttttgaagccatttaaagataacctgataaaaatacccaacgttaaaaatacatacaagtggataggagttaatggacaaactgattcattatccctcccgagtaaggtcttatctatctctaataaaaaggttgattcttctacagttcttcttgtgcatgataagaagggtggtaagcggaattgggtacctaaaagtatagggggtttgttggtatatataactcctaaagacgggccgaaaagaatagttcaagtaaagccagcaactcagggtttgttaatatatagaaatgaagatggcagagtagatgctgtaagagagttcataaatccagtgatagaaatgtataataatggcaaattggcatttgtagaaaaagaaaatgaagaagagcttttgaaatattttaatttgctggaaaaaggtcaaaaatttgaaagaataagacggtatgatatgataacctacaatagtaaattttactatgtaacaaaaataaacaagaatcacagagttactatacaagaagagtctaagataaaagcagaatcagacaaagttaagtcctcttcaggcaaagagtatactcgtaaggaaaccgaggaattatcacttcaaaaattagcggaattaattagtatataaaa

SEQ ID NO: 5 - protein, 978 aa, Artificial Sequence
  Description of Artificial Sequence Synthetic polypeptide
  CasX.1
MQEIKRINKIRRRLVKDSNTKKAGKTGPMKTLLVRVMTPDLRERLENLRKKPENIPQPISNTSRANLNKLLTDYTEMKKAILHVYWEEFQKDPVGLMSRVAQPAPKNIDQRKLIPVKDGNERLTSSGFACSQCCQPLYVYKLEQVNDKGKPHTNYFGRCNVSEHERLILLSPHKPEANDELVTYSLGKFGQRALDFYSIHVTRESNHPVKPLEQIGGNSCASGPVGKALSDACMGAVASFLTKYQDIILEHQKVIKKNEKRLANLKDIASANGLAFPKITLPPQPHTKEGIEAYNNVVAQIVIWVNLNLWQKLKIGRDEAKPLQRLKGFPSFPLVERQANEVDWWDMVCNVKKLINEKKEDGKVFWQNLAGYKRQEALLPYLSSEEDRKKGKKFARYQFGDLLLHLEKKHGEDWGKVYDEAWERIDKKVEGLSKHIKLEEERRSEDAQSKAALTDWLRAKASFVIEGLKEADKDEFCRCELKLQKWYGDLRGKPFAIEAENSILDISGFSKQYNCAFIWQKDGVKKLNLYLIINYFKGGKLRFKKIKPEAFEANRFYTVINKKSGEIVPMEVNFNFDDPNLIILPLAFGKRQGREFIWNDLLSLETGSLKLANGRVIEKTLYNRRTRQDEPALFVALTFERREVLDSSNIKPMNLIGIDRGENIPAVIALTDPEGCPLSRFKDSLGNPTHILRIGESYKEKQRTIQAAKEVEQRRAGGYSRKYASKAKNLADDMVRNTARDLLYYAVTQDAMLIFENLSRGFGRQGKRTFMAERQYTRMEDWLTAKLAYEGLPSKTYLSKTLAQYTSKTCSNCGFTITSADYDRVLEKLKKTATGWMTTINGKELKVEGQITYYNRYKRQNVVKDLSVELDRLSEESVNNDISSWTKGRSGEALSLLKKRFSHRPVQEKFVCLNCGFETHADEQAALNIARSWLFLRSQEYKKYQTNKTTGNTDKRAFVETWQSFYRKKLKEVWKPAV

SEQ ID NO: 6 - DNA, 5495 bp, Artificial Sequence
  Description of Artificial Sequence Synthetic polynucleotide
  CasX.1
atgcttcttatttatcggagatatcttcaaacaccatcaacatggcaatggtgaaccattaatattctttgatgcttcttatttatcggagatatcttcaaacattgcccattttacaggcatatcttctggctctttgatgcttcttatttatcggagatatcttcaaacgtaatgtattgagaaagacatcaagattagataactttgatgcttcttatttatcggagatatcttcaaacacagaaacctgcaaagattgtatatatataagctttgatgcttcttatttatcggagatatcttcaaacgatacgtattttagcccgtctatttggggattaactttgatgcttcttatttatcggagatatcttcaaaccccgcatatccagatttttcaatgacttctggaaattgtattttcaatattttacaagttgcggaggatacctttaataatttagcagagttacgcactgtaaacctgttcttctcacaaaaagctttaacatcagattttcaaagaacttcttatgtaatttataagaatctaaaaaaacagctctgggtttgcatccagaactctccgataaataagcgctttacccatacgacatagtcgctggtgatggctctcaaagtaatgagataaaagcgccagtaataatttactattcacaaatcctttcgtcaagcttaaaatcaatcaaagaccatatccccttcattccaaatagcagcgcttccgtacctttctatccgttcatatatctcctctgagagaggataaattaccagacttatagagccatccataaatcctttttctttaaggttgagctttagatcagcccaccttgcttttgaaaggttaaactcaaagacagaatattgaatccgaacaccataggcttccagaagtttaactaaccgtgccctgaccttatcatcttcaatatcataacaaatgagatgtcgcattttaaagctctataggcttataacattccctatcatcttgaatatgctggctaaacaacctaacctgccgctcaactgcgtgctgatacgttattgattggataagtaaattggttttctgctcatctaccttaaagaattgatgccattttttgattacttttggataggcatccttattcagccaaacacctttttggtcagtttctttcctgaaatcgtctgtatccacttcccttctatttatcaaattgatcacaaaacggtcagccaacggccgccactcctccagaagatcgcatattaaagagggacgaccataatagacgtcatgcaagtaaccaaaggccgggtcaaaaccgacgagtaatgcagtcgaatgtatttcgttgaacaggagggtgtagataaggctcatcatggcgttgatttcatcctcaggaggtctcttggtacggcgcacaaaaacaaagcttggatgctttaagatagccgaaaaattgccataatactgccttgttgttgcgccttctattccacgcaaggtctctaaatcagtgacggcgttgatttcggtacactcgattctcaaaccaagtctatatttatcaagtaatgattgctggtttttgatcttaccggcaacgatactttttgcaatttcaagttttttgtggggatcaaaatgcttatgaatttgcgcccgacgaataaacagatttttgacgggttcaaattgaaggctcccttgatattcccatctgccgctaaagaaatgtatcggtatagattattctctgcaaaggctaataacacggctatcgagggtaacccggccaactaccacgatatcttttaccttcattgcgggaatcttctgccccttctcttcattgtccttttttatgagaaatgcccgaccacgacaatccaaaatgaattcatcacccgtgagatagagggttatcctgtcggttatagcggtcatcagtaagccttttatttttctaaccaagtattgaaggaagacacgattcactatactggcactgcggacacctatggtcatcaaccttgggaaacctgcttatatcaaaggacaagaagcagtctcgcagatttgtaacaacttctacacaacgcactttcagggttttatctataacaatttctttccgtctccgtgtttcacagaaaaatatttcaccaactggtatattgacattatacatctcttcaaggcaaattgcctgtaacccaatctgaacgtggaagttctcaaaatcccttaccttccctgtctttgtttcgataggaatcggtatcccatccctccactcgataaggtctgcccggcctgccaaaccgagcttattgctgtaaagatacacgcctgttacctgcttacaatcagggcagcttctctgcgatgatttatccaccgccctgtgcgcgtgtatggcctctgtaaagtggatgctcttagccatattacgccgttctccaacaaaggcataccatgcattgcgcggacaatagattgactccattaccgtgctgatgtgcaatatcagacggctggtttccatacttctttgagcttctttctgtaaaaggattgccatgtttcaacaaatgcccttttgtcagtatttccggtcgttttattggtttgatacttcttatattcttgagaacggagaaagagccacgaccttgcaatattcagtgctgcttgttcgtctgcatgggtttcaaaaccacagttcaggcaaacaaacttttcctgcaccggcctgtgactaaatctcttttttagcagagataaagcttcaccactgcggccttttgtccaactagaaatatcattatttaccgactcttccgaaagtctatccagctctacagagaggtcttttaccacattctgccttttataccggttatagtatgttatctgtccttcaacttttaactcttttccattgattgtagtcatccatccagtagccgtcttcttgagcttttcgagcaccctgtcataatctgcacttgtgattgtaaaaccacaattagaacatgtctttgaggtatactgtgccagagtctttgaaagataggtttttgatggcagaccttcataggcaagctttgcagtcagccagtcttccatcctcgtgtactgcctttccgccataaaagtcctcttgccttgtctaccaaaaccgcgggaaagattttcaaaaatgagcattgcatcttgagtaacagcataatataagaggtcacgagctgtatttcttaccatatcgtccgccagattcttcgcctttgatgcatattttctcgaatatccgcctgcccgcctttgttcaacttctttagcagcctgaatagtccgttgtttttccttataactttctcctattcgcaaaatatgcgttggattgcccaatgaatctttgaatcttgacaaggggcatccttccgggtctgttaatgctatgactgccgggatattttctccccggtctattcctatcagattcatcggttttatattcgatgagtcaagcacctctcttctttcaaatgtcagggcaacaaaaagtgctggttcatcctgtctcgtccttctgttatagagcgttttttcaataaccctgccattggcgagtttcaatgaacccgtctcaaggctcaataggtcgttccagataaactccctcccctgcctttttccaaaggccaaaggcagaattatcaaattcgggtcatcaaaattgaagttgacctccataggcacaatctcaccgctttttttattaattactgtataaaacctatttgcttcaaaagcttctggcttgatttttttgaagcgtagcttaccacctttgaagtaatttattattaaataaagatttaacttctttacgccgtctttctgccatataaatgcacaattatactgtttagaaaatccgcttatatctaaaatgctgttctctgcttctatagcaaatggttttcctctcaaatctccataccacttttgaagctttaactcacacctgcaaaactcatccttatcagcttctttgagcccttcaataacaaaagaggcctttgccctgagccaatcagtgagggcagcctttgattgagcatcttcagaccttctttcttcctccaactttatgtgcttactcagaccttcaacttttttatctattctttcccatgcctcatcataaactttgccccaatcttcaccgtgtttcttttcaaggtgaagcaaaaggtcaccaaactgataacgcgcaaacttttttccttttttacggtcttcttcagacgaaagatatggaagcaaggcttcctgccttttatatccagcaagattttgccagaagaccttcccgtcctctttcttttcgttaatcaactttttgacattacagaccatatcccaccaatcaacctcattcgcctggcgttcaacaagagggaaggacggaaaacccttaagccgctgtaagggctttgcctcatccctgccaattttgagtttctgccaaagattcaggtttacccagatcactatctgagcaacaacattgttataagcttcaatcccttcttttgtatgcggttgcggtggaagagtgattttaggaaatgcaagcccgtttgcacttgctatatcctttagatttgccaatctcttttcgtttttttttataaccttttggtgttcgaggatgatgtcctggtactttgtaaggaaactggctactgctcccatacaggcatcagataaagccttaccaacgggaccacttgcgcagctattgccaccgatctgttctagcggctttacaggatggttcgattctcttgttacgtggattgaataaaagtccaatgccctttgaccgaacttccccaacgaatacgttactagctcgtcatttgcctccggtttatgcggcgagagcaatatcaaacgttcatgctcggagacattacaacggccaaagtaatttgtatggggcttacccttgtcattcacttgttcaagcttataaacatagaggggttgacagcactgagaacaggcaaatccagaacttgttagtctctcatttccgtccttcaccggaatcaattttctctgatcaatattcttgggcgctggttgtgcaaccctgctcatcaatccgacagggtctttttggaactcttcccaataaacatgcaggattgctttcttcatttccgtatagtcagtgaggagtttatttaaatttgcacgtgaagtatttgaaatgggctgaggaatgttttccggctttttgcgaagattctctaacctttctctcaggtcaggtgtcataacccgaacgagcaaggttttcatagggccggttttgccggcttttttcgtgttgctatcctttaccaatctccttcgtattttatttatcctttttatttcctgcatcttt

SEQ ID NO: 7 - protein, 986 aa, Artificial Sequence
  Description of Artificial Sequence Synthetic polypeptide
  CasX.1 deltaproteobacteria
MEKRINKIRKKLSADNATKPVSRSGPMKTLLVRVMTDDLKKRLEKRRKKPEVMPQVISNNAANNLRMLLDDYTKMKEAILQVYWQEFKDDHVGLMCKFAQPASKKIDQNKLKPEMDEKGNLTTAGFACSQCGQPLFVYKLEQVSEKGKAYTNYFGRCNVAEHEKLILLAQLKPEKDSDEAVTYSLGKFGQRALDFYSIHVTKESTHPVKPLAQIAGNRYASGPVGKALSDACMGTIASFLSKYQDIIIEHQKVVKGNQKRLESLRELAGKENLEYPSVTLPPQPHTKEGVDAYNEVIARVRMWVNLNLWQKLKLSRDDAKPLLRLKGFPSFPVVERRENEVDWWNTINEVKKLIDAKRDMGRVFWSGVTAEKRNTILEGYNYLPNENDHKKREGSLENPKKPAKRQFGDLLLYLEKKYAGDWGKVFDEAWERIDKKIAGLTSHIEREEARNAEDAQSKAVLTDWLRAKASFVLERLKEMDEKEFYACEIQLQKWYGDLRGNPFAVEAENRVVDISGFSIGSDGHSIQYRNLLAWKYLENGKREFYLLMNYGKKGRIRFTDGTDIKKSGKWQGLLYGGGKAKVIDLTFDPDDEQLIILPLAFGTRQGREFIWNDLLSLETGLIKLANGRVIEKTIYNKKIGRDEPALFVALTFERREVVDPSNIKPVNLIGVDRGENIPAVIALTDPEGCPLPEFKDSSGGPTDILRIGEGYKEKQRAIQAAKEVEQRRAGGYSRKFASKSRNLADDMVRNSARDLFYHAVTHDAVLVFENLSRGFGRQGKRTFMTERQYTKMEDWLTAKLAYEGLTSKTYLSKTLAQYTSKTCSNCGFTITTADYDGMLVRLKKTSDGWATTLNNKELKAEGQITYYNRYKRQTVEKELSAELDRLSEESGNNDISKWTKGRRDEALFLLKKRFSHRPVQEQFVCLDCGHEVHADEQAALNIARSWLFLNSNSTEFKSYKSGKQPFVGAWQAFYKRRLKEVWKPNA

SEQ ID NO: 8 - DNA, 2962 bp, Artificial Sequence
  Description of Artificial Sequence Synthetic polynucleotide
  CasX.1 deltaproteobacteria
atggaaaagagaataaacaagatacgaaagaaactatcggccgataatgccacaaagcctgtgagcaggagcggccccatgaaaacactccttgtccgggtcatgacggacgacttgaaaaaaagactggagaagcgtcggaaaaagccggaagttatgccgcaggttatttcaaataacgcagcaaacaatcttagaatgctccttgatgactatacaaagatgaaggaggcgatactacaagtttactggcaggaatttaaggacgaccatgtgggcttgatgtgcaaatttgcccagcctgcttccaaaaaaattgaccagaacaaactaaaaccggaaatggatgaaaaaggaaatctaacaactgccggttttgcatgttctcaatgcggtcagccgctatttgtttataagcttgaacaggtgagtgaaaaaggcaaggcttatacaaattacttcggccggtgtaatgtggccgagcatgagaaattgattcttcttgctcaattaaaacctgaaaaagacagtgacgaagcagtgacatactcccttggcaaattcggccagagggcattggacttttattcaatccacgtaacaaaagaatccacccatccagtaaagcccctggcacagattgcgggcaaccgctatgcaagcggacctgttggcaaggccctttccgatgcctgtatgggcactatagccagttttctttcgaaatatcaagacatcatcatagaacatcaaaaggttgtgaagggtaatcaaaagaggttagagagtctcagggaattggcagggaaagaaaatcttgagtacccatcggttacactgccgccgcagccgcatacgaaagaaggggttgacgcttataacgaagttattgcaagggtacgtatgtgggttaatcttaatctgtggcaaaagctgaagctcagccgtgatgacgcaaaaccgctactgcggctaaaaggattcccatctttccctgttgtggagcggcgtgaaaacgaagttgactggtggaatacgattaatgaagtaaaaaaactgattgacgctaaacgagatatgggacgggtattctggagcggcgttaccgcagaaaagagaaataccatccttgaaggatacaactatctgccaaatgagaatgaccataaaaagagagagggcagtttggaaaaccctaagaagcctgccaaacgccagtttggagacctcttgctgtatcttgaaaagaaatatgccggagactggggaaaggtcttcgatgaggcatgggagaggatagataagaaaatagccggactcacaagccatatagagcgcgaagaagcaagaaacgcggaagacgctcaatccaaagccgtacttacagactggctaagggcaaaggcatcatttgttcttgaaagactgaaggaaatggatgaaaaggaattctatgcgtgtgaaatccaacttcaaaaatggtatggcgatcttcgaggcaacccgtttgccgttgaagctgagaatagagttgttgatataagcgggttttctatcggaagcgatggccattcaatccaatacagaaatctccttgcctggaaatatctggagaacggcaagcgtgaattctatctgttaatgaattatggcaagaaagggcgcatcagatttacagatggaacagatattaaaaagagcggcaaatggcagggactattatatggcggtggcaaggcaaaggttattgatctgactttcgaccccgatgatgaacagttgataatcctgccgctggcctttggcacaaggcaaggccgcgagtttatctggaacgatttgctgagtcttgaaacaggcctgataaagctcgcaaacggaagagttatcgaaaaaacaatctataacaaaaaaatagggcgggatgaaccggctctattcgttgccttaacatttgagcgccgggaagttgttgatccatcaaatataaagcctgtaaaccttataggcgttgaccgcggcgaaaacatcccggcggttattgcattgacagaccctgaaggttgtcctttaccggaattcaaggattcatcagggggcccaacagacatcctgcgaataggagaaggatataaggaaaagcagagggctattcaggcagcaaaggaggtagagcaaaggcgggctggcggttattcacggaagtttgcatccaagtcgaggaacctggcggacgacatggtgagaaattcagcgcgagaccttttttaccatgccgttacccacgatgccgtccttgtctttgaaaacctgagcaggggttttggaaggcagggcaaaaggaccttcatgacggaaagacaatatacaaagatggaagactggctgacagcgaagctcgcatacgaaggtcttacgtcaaaaacctacctttcaaagacgctggcgcaatatacgtcaaaaacatgctccaactgcgggtttactataacgactgccgattatgacgggatgttggtaaggcttaaaaagacttctgatggatgggcaactaccctcaacaacaaagaattaaaagccgaaggccagataacgtattataaccggtataaaaggcaaaccgtggaaaaagaactctccgcagagcttgacaggctttcagaagagtcgggcaataatgatatttctaagtggaccaagggtcgccgggacgaggcattatttttgttaaagaaaagattcagccatcggcctgttcaggaacagtttgtttgcctcgattgcggccatgaagtccacgccgatgaacaggcagccttgaatattgcaaggtcatggctttttctaaactcaaattcaacagaattcaaaagttataaatcgggtaaacagcccttcgttggtgcttggcaggccttttacaaaaggaggcttaaagaggtatggaagcccaacgcctgat

SEQ ID NO: 9 - protein, 1125 aa, Artificial Sequence
  Description of Artificial Sequence Synthetic polypeptide
  CasY.1
MRKKLFKGYILHNKRLVYTGKAAIRSIKYPLVAPNKTALNNLSEKIIYDYEHLFGPLNVASYARNSNRYSLVDFWIDSLRAGVIWQSKSTSLIDLISKLEGSKSPSEKIFEQIDFELKNKLDKEQFKDIILLNTGIRSSSNVRSLRGRFLKCFKEEFRDTEEVIACVDKWSKDLIVEGKSILVSKQFLYWEEEFGIKIFPHFKDNHDLPKLTFFVEPSLEFSPHLPLANCLERLKKFDISRESLLGLDNNFSAFSNYFNELFNLLSRGEIKKIVTAVLAVSKSWENEPELEKRLHFLSEKAKLLGYPKLTSSWADYRMIIGGKIKSWHSNYTEQLIKVREDLKKHQIALDKLQEDLKKVVDSSLREQIEAQREALLPLLDTMLKEKDFSDDLELYRFILSDFKSLLNGSYQRYIQTEEERKEDRDVTKKYKDLYSNLRNIPRFFGESKKEQFNKFINKSLPTIDVGLKILEDIRNALETVSVRKPPSITEEYVTKQLEKLSRKYKINAFNSNRFKQITEQVLRKYNNGELPKISEVFYRYPRESHVAIRILPVKISNPRKDISYLLDKYQISPDWKNSNPGEVVDLIEIYKLTLGWLLSCNKDFSMDFSSYDLKLFPEAASLIKNFGSCLSGYYLSKMIFNCITSEIKGMITLYTRDKFVVRYVTQMIGSNQKFPLLCLVGEKQTKNFSRNWGVLIEEKGDLGEEKNQEKCLIFKDKTDFAKAKEVEIFKNNIWRIRTSKYQIQFLNRLFKKTKEWDLMNLVLSEPSLVLEEEWGVSWDKDKLLPLLKKEKSCEERLYYSLPLNLVPATDYKEQSAEIEQRNTYLGLDVGEFGVAYAVVRIVRDRIELLSWGFLKDPALRKIRERVQDMKKKQVMAVFSSSSTAVARVREMAIHSLRNQIHSIALAYKAKIIYEISISNFETGGNRMAKIYRSIKVSDVYRESGADTLVSEMIWGKKNKQMGNHISSYATSYTCCNCARTPFELVIDNDKEYEKGGDEFIFNVGDEKKVRGFLQKSLLGKTIKGKEVLKSIKEYARPPIREVLLEGEDVEQLLKRRGNSYIYRCPFCGYKTDADIQAALNIACRGYISDNAKDAVKEGERKLDYILEVRKLWEKNGAVLRSAKFL

SEQ ID NO: 10 - DNA, 3380 bp, Artificial Sequence
  Description of Artificial Sequence Synthetic polynucleotide
  CasY.1
atgcgcaaaaaattgtttaagggttacattttacataataagaggcttgtatatacaggtaaagctgcaatacgttctattaaatatccattagtcgctccaaataaaacagccttaaacaatttatcagaaaagataatttatgattatgagcatttattcggacctttaaatgtggctagctatgcaagaaattcaaacaggtacagccttgtggatttttggatagatagcttgcgagcaggtgtaatttggcaaagcaaaagtacttcgctaattgatttgataagtaagctagaaggatctaaatccccatcagaaaagatatttgaacaaatagattttgagctaaaaaataagttggataaagagcaattcaaagatattattcttcttaatacaggaattcgttctagcagtaatgttcgcagtttgagggggcgctttctaaagtgttttaaagaggaatttagagataccgaagaggttatcgcctgtgtagataaatggagcaaggaccttatcgtagagggtaaaagtatactagtgagtaaacagtttctttattgggaagaagagtttggtattaaaatttttcctcattttaaagataatcacgatttaccaaaactaactttttttgtggagccttccttggaatttagtccgcacctccctttagccaactgtcttgagcgtttgaaaaaattcgatatttcgcgtgaaagtttgctcgggttagacaataatttttcggccttttctaattatttcaatgagctttttaacttattgtccaggggggagattaaaaagattgtaacagctgtccttgctgtttctaaatcgtgggagaatgagccagaattggaaaagcgcttacattttttgagtgagaaggcaaagttattagggtaccctaagcttacttcttcgtgggcggattatagaatgattattggcggaaaaattaaatcttggcattctaactataccgaacaattaataaaagttagagaggacttaaagaaacatcaaatcgcccttgataaattacaggaagatttaaaaaaagtagtagatagctctttaagagaacaaatagaagctcaacgagaagctttgcttcctttgcttgataccatgttaaaagaaaaagatttttccgatgatttagagctttacagatttatcttgtcagattttaagagtttgttaaatgggtcttatcaaagatatattcaaacagaagaggagagaaaggaggacagagatgttaccaaaaaatataaagatttatatagtaatttgcgcaacatacctagattttttggggaaagtaaaaaggaacaattcaataaatttataaataaatctctcccgaccatagatgttggtttaaaaatacttgaggatattcgtaatgctctagaaactgtaagtgttcgcaaacccccttcaataacagaagagtatgtaacaaagcaacttgagaagttaagtagaaagtacaaaattaacgcctttaattcaaacagatttaaacaaataactgaacaggtgctcagaaaatataataacggagaactaccaaagatctcggaggttttttatagatacccgagagaatctcatgtggctataagaatattacctgttaaaataagcaatccaagaaaggatatatcttatcttctcgacaaatatcaaattagccccgactggaaaaacagtaacccaggagaagttgtagatttgatagagatatataaattgacattgggttggctcttgagttgtaacaaggatttttcgatggatttttcatcgtatgacttgaaactcttcccagaagccgcttccctcataaaaaattttggctcttgcttgagtggttactatttaagcaaaatgatatttaattgcataaccagtgaaataaaggggatgattactttatatactagagacaagtttgttgttagatatgttacacaaatgataggtagcaatcagaaatttcctttgttatgtttggtgggagagaaacagactaaaaacttttctcgcaactggggtgtattgatagaagagaagggagatttgggggaggaaaaaaaccaggaaaaatgtttgatatttaaggataaaacagattttgctaaagctaaagaagtagaaatttttaaaaataatatttggcgtatcagaacctctaagtaccaaatccaatttttgaataggctttttaagaaaaccaaagaatgggatttaatgaatcttgtattgagcgagcctagcttagtattggaggaggaatggggtgtttcgtgggataaagataaacttttacctttactgaagaaagaaaaatcttgcgaagaaagattatattactcacttccccttaacttggtgcctgccacagattataaggagcaatctgcagaaatagagcaaaggaatacatatttgggtttggatgttggagaatttggtgttgcctatgcagtggtaagaatagtaagggacagaatagagcttctgtcctggggattccttaaggacccagctcttcgaaaaataagagagcgtgtacaggatatgaagaaaaagcaggtaatggcagtattttctagctcttccacagctgtcgcgcgagtacgagaaatggctatacactctttaagaaatcaaattcatagcattgctttggcgtataaagcaaagataatttatgagatatctataagcaattttgagacaggtggtaatagaatggctaaaatataccgatctataaaggtttcagatgtttatagggagagtggtgcggataccctagtttcagagatgatctggggcaaaaagaataagcaaatgggaaaccatatatcttcctatgcgacaagttacacttgttgcaattgtgcaagaaccccttttgaacttgttatagataatgacaaggaatatgaaaagggaggcgacgaatttatttttaatgttggcgatgaaaagaaggtaagggggtttttacaaaagagtctgttaggaaaaacaattaaagggaaggaagtgttgaagtctataaaagagtacgcaaggccgcctataagggaagtcttgcttgaaggagaagatgtagagcagttgttgaagaggagaggaaatagctatatttatagatgccctttttgtggatataaaactgatgcggatattcaagcggcgttgaatatagcttgtaggggatatatttcggataacgcaaaggatgctgtgaaggaaggagaaagaaaattagattacattttggaagttagaaaattgtgggagaagaatggagctgttttgagaagcgccaaatttttatagtt

SEQ ID NO: 11 - protein, 1226 aa, Artificial Sequence
  Description of Artificial Sequence Synthetic polypeptide
  CasY.2
MQKVRKTLSEVHKNPYGTKVRNAKTGYSLQIERLSYTGKEGMRSFKIPLENKNKEVFDEFVKKIRNDYISQVGLLNLSDWYEHYQEKQEHYSLADFWLDSLRAGVIFAHKETEIKNLISKIRGDKSIVDKFNASIKKKHADLYALVDIKALYDFLTSDARRGLKTEEEFFNSKRNTLFPKFRKKDNKAVDLWVKKFIGLDNKDKLNFTKKFIGFDPNPQIKYDHTFFFHQDINFDLERITTPKELISTYKKFLGKNKDLYGSDETTEDQLKMVLGFHNNHGAFSKYFNASLEAFRGRDNSLVEQIINNSPYWNSHRKELEKRIIFLQVQSKKIKETELGKPHEYLASFGGKFESWVSNYLRQEEEVKRQLFGYEENKKGQKKFIVGNKQELDKIIRGTDEYEIKAISKETIGLTQKCLKLLEQLKDSVDDYTLSLYRQLIVELRIRLNVEFQETYPELIGKSEKDKEKDAKNKRADKRYPQIFKDIKLIPNFLGETKQMVYKKFIRSADILYEGINFIDQIDKQITQNLLPCFKNDKERIEFTEKQFETLRRKYYLMNSSRFHHVIEGIINNRKLIEMKKRENSELKTFSDSKFVLSKLFLKKGKKYENEVYYTFYINPKARDQRRIKIVLDINGNNSVGILQDLVQKLKPKWDDIIKKNDMGELIDAIEIEKVRLGILIALYCEHKFKIKKELLSLDLFASAYQYLELEDDPEELSGTNLGRFLQSLVCSEIKGAINKISRTEYIERYTVQPMNTEKNYPLLINKEGKATWHIAAKDDLSKKKGGGTVAMNQKIGKNFFGKQDYKTVFMLQDKRFDLLTSKYHLQFLSKTLDTGGGSWWKNKNIDLNLSSYSFIFEQKVKVEWDLTNLDHPIKIKPSENSDDRRLFVSIPFVIKPKQTKRKDLQTRVNYMGIDIGEYGLAWTIINIDLKNKKINKISKQGFIYEPLTHKVRDYVATIKDNQVRGTFGMPDTKLARLRENAITSLRNQVHDIAMRYDAKPVYEFEISNFETGSNKVKVIYDSVKRADIGRGQNNTEADNTEVNLVWGKTSKQFGSQIGAYATSYICSFCGYSPYYEFENSKSGDEEGARDNLYQMKKLSRPSLEDFLQGNPVYKTFRDFDKYKNDQRLQKTGDKDGEWKTHRGNTAIYACQKCRHISDADIQASYWIALKQVVRDFYKDKEMDGDLIQGDNKDKRKVNELNRLIGVHKDVPIINKNLITSLDINLL

SEQ ID NO: 12 - DNA, 2869 bp, Artificial Sequence
  Description of Artificial Sequence Synthetic polynucleotide
  CasY.2
atggtattaggttttcataataatcacggcgctttttctaagtatttcaacgcgagcttggaagcttttagggggagagacaactccttggttgaacaaataattaataattctccttactggaatagccatcggaaagaattggaaaagagaatcatttttttgcaagttcagtctaaaaaaataaaagagaccgaactgggaaagcctcacgagtatcttgcgagttttggcgggaagtttgaatcttgggtttcaaactatttacgtcaggaagaagaggtcaaacgtcaactttttggttatgaggagaataaaaaaggccagaaaaaatttatcgtgggcaacaaacaagagctagataaaatcatcagagggacagatgagtatgagattaaagcgatttctaaggaaaccattggacttactcagaaatgtttaaaattacttgaacaactaaaagatagtgtcgatgattatacacttagcctatatcggcaactcatagtcgaattgagaatcagactgaatgttgaattccaagaaacttatccggaattaatcggtaagagtgagaaagataaagaaaaagatgcgaaaaataaacgggcagacaagcgttacccgcaaatttttaaggatataaaattaatccccaattttctcggtgaaacgaaacaaatggtatataagaaatttattcgttccgctgacatcctttatgaaggaataaattttatcgaccagatcgataaacagattactcaaaatttgttgccttgttttaagaacgacaaggaacggattgaatttaccgaaaaacaatttgaaactttacggcgaaaatactatctgatgaatagttcccgttttcaccatgttattgaaggaataatcaataataggaaacttattgaaatgaaaaagagagaaaatagcgagttgaaaactttctccgatagtaagtttgttttatctaagctttttcttaaaaaaggcaaaaaatatgaaaatgaggtctattatactttttatataaatccgaaagctcgtgaccagcgacggataaaaattgttcttgatataaatgggaacaattcagtcggaattttacaagatcttgtccaaaagttgaaaccaaaatgggacgacatcataaagaaaaatgatatgggagaattaatcgatgcaatcgagattgagaaagtccggctcggcatcttgatagcgttatactgtgagcataaattcaaaattaaaaaagaactcttgtcattagatttgtttgccagtgcctatcaatatctagaattggaagatgaccctgaagaactttctgggacaaacctaggtcggtttttacaatccttggtctgctccgaaattaaaggtgcgattaataaaataagcaggacagaatatatagagcggtatactgtccagccgatgaatacggagaaaaactatcctttactcatcaataaggagggaaaagccacttggcatattgctgctaaggatgacttgtccaagaagaagggtgggggcactgtcgctatgaatcaaaaaatcggcaagaatttttttgggaaacaagattataaaactgtgtttatgcttcaggataagcggtttgatctactaacctcaaagtatcacttgcagtttttatctaaaactcttgatactggtggagggtcttggtggaaaaacaaaaatattgatttaaatttaagctcttattctttcattttcgaacaaaaagtaaaagtcgaatgggatttaaccaatcttgaccatcctataaagattaagcctagcgagaacagtgatgatagaaggcttttcgtatccattccttttgttattaaaccgaaacagacaaaaagaaaggatttgcaaactcgagtcaattatatggggattgatatcggagaatatggtttggcttggacaattattaatattgatttaaagaataaaaaaataaataagatttcaaaacaaggtttcatctatgagccgttgacacataaagtgcgcgattatgttgctaccattaaagataatcaggttagaggaacttttggcatgcctgatacgaaactagccagattgcgagaaaatgccattaccagcttgcgcaatcaagtgcatgatattgctatgcgctatgacgccaaaccggtatatgaatttgaaatttccaattttgaaacggggtctaataaagtgaaagtaatttatgattcggttaagcgagctgatatcggccgaggccagaataataccgaagcagacaatactgaggttaatcttgtctgggggaagacaagcaaacaatttggcagtcaaatcggcgcttatgcgacaagttacatctgttcattttgtggttattctccatattatgaatttgaaaattctaagtcgggagatgaagaaggggctagagataatctatatcagatgaagaaattgagtcgcccctctcttgaagatttcctccaaggaaatccggtttataagacatttagggattttgataagtataaaaacgatcaacggttgcaaaagacgggtgataaagatggtgaatggaaaacacacagagggaatactgcaatatacgcctgtcaaaagtgtagacatatctctgatgcggatatccaagcatcatattggattgctttgaagcaagttgtaagagatttttataaagacaaagagatggatggtgatttgattcaaggagataataaagacaagagaaaagtaaacgagcttaatagacttattggagtacataaagatgtgcctataataaataaaaatttaataacatcactcgacataaacttactataga

SEQ ID NO: 13 - protein, 1200 aa, Artificial Sequence
  Description of Artificial Sequence Synthetic polypeptide
  CasY.3
MKAKKSFYNQKRKFGKRGYRLHDERIAYSGGIGSMRSIKYELKDSYGIAGLRNRIADATISDNKWLYGNINLNDYLEWRSSKTDKQIEDGDRESSLLGFWLEALRLGFVFSKQSHAPNDFNETALQDLFETLDDDLKHVLDRKKWCDFIKIGTPKTNDQGRLKKQIKNLLKGNKREEIEKTLNESDDELKEKINRIADVFAKNKSDKYTIFKLDKPNTEKYPRINDVQVAFFCHPDFEEITERDRTKTLDLIINRFNKRYEITENKKDDKTSNRMALYSLNQGYIPRVLNDLFLFVKDNEDDFSQFLSDLENFFSFSNEQIKIIKERLKKLKKYAEPIPGKPQLADKWDDYASDFGGKLESWYSNRIEKLKKIPESVSDLRNNLEKIRNVLKKQNNASKILELSQKIIEYIRDYGVSFEKPEIIKFSWINKTKDGQKKVFYVAKMADREFIEKLDLWMADLRSQLNEYNQDNKVSFKKKGKKIEELGVLDFALNKAKKNKSTKNENGWQQKLSESIQSAPLFFGEGNRVRNEEVYNLKDLLFSEIKNVENILMSSEAEDLKNIKIEYKEDGAKKGNYVLNVLARFYARFNEDGYGGWNKVKTVLENIAREAGTDFSKYGNNNNRNAGRFYLNGRERQVFTLIKFEKSITVEKILELVKLPSLLDEAYRDLVNENKNHKLRDVIQLSKTIMALVLSHSDKEKQIGGNYIHSKLSGYNALISKRDFISRYSVQTTNGTQCKLAIGKGKSKKGNEIDRYFYAFQFFKNDDSKINLKVIKNNSHKNIDFNDNENKINALQVYSSNYQIQFLDWFFEKHQGKKTSLEVGGSFTIAEKSLTIDWSGSNPRVGFKRSDTEEKRVFVSQPFTLIPDDEDKERRKERMIKTKNRFIGIDIGEYGLAWSLIEVDNGDKNNRGIRQLESGFITDNQQQVLKKNVKSWRQNQIRQTFTSPDTKIARLRESLIGSYKNQLESLMVAKKANLSFEYEVSGFEVGGKRVAKIYDSIKRGSVRKKDNNSQNDQSWGKKGINEWSFETTAAGTSQFCTHCKRWSSLAIVDIEEYELKDYNDNLFKVKINDGEVRLLGKKGWRSGEKIKGKELFGPVKDAMRPNVDGLGMKIVKRKYLKLDLRDWVSRYGNMAIFICPYVDCHHISHADKQAAFNIAVRGYLKSVNPDRAIKHGDKGLSRDFLCQEEGKLNFEQIGLL

SEQ ID NO: 14 - DNA, 3604 bp, Artificial Sequence
  Description of Artificial Sequence Synthetic polynucleotide
  CasY.3
atgaaagctaaaaaaagtttttataatcaaaagcggaagttcggtaaaagaggttatcgtcttcacgatgaacgtatcgcgtattcaggagggattggatcgatgcgatctattaaatatgaattgaaggattcgtatggaattgctgggcttcgtaatcgaatcgctgacgcaactatttctgataataagtggctgtacgggaatataaatctaaatgattatttagagtggcgatcttcaaagactgacaaacagattgaagacggagaccgagaatcatcactcctgggtttttggctggaagcgttacgactgggattcgtgttttcaaaacaatctcatgctccgaatgattttaacgagaccgctctacaagatttgtttgaaactcttgatgatgatttgaaacatgttcttgataggaaaaaatggtgtgactttatcaagataggaacacctaagacaaatgaccaaggtcgtttaaaaaaacaaatcaagaatttgttaaaaggaaacaagagagaggaaattgaaaaaactctcaatgaatcagacgatgaattgaaagagaaaataaacagaattgccgatgtttttgcaaaaaataagtctgataaatacacaattttcaaattagataaacccaatacggaaaaataccccagaatcaacgatgttcaggtggcgtttttttgtcatcccgattttgaggaaattacagaacgagatagaacaaagactctagatctgatcattaatcggtttaataagagatatgaaattaccgaaaataaaaaagatgacaaaacttcaaacaggatggccttgtattccttgaaccagggctatattcctcgcgtcctgaatgatttattcttgtttgtcaaagacaatgaggatgattttagtcagtttttatctgatttggagaatttcttctctttttccaacgaacaaattaaaataataaaggaaaggttaaaaaaacttaaaaaatatgctgaaccaattcccggaaagccgcaacttgctgataaatgggacgattatgcttctgattttggcggtaaattggaaagctggtactccaatcgaatagagaaattaaagaagattccggaaagcgtttccgatctgcggaataatttggaaaagatacgcaatgttttaaaaaaacaaaataatgcatctaaaatcctggagttatctcaaaagatcattgaatacatcagagattatggagtttcttttgaaaagccggagataattaagttcagctggataaataagacgaaggatggtcagaaaaaagttttctatgttgcgaaaatggcggatagagaattcatagaaaagcttgatttatggatggctgatttacgcagtcaattaaatgaatacaatcaagataataaagtttctttcaaaaagaaaggtaaaaaaatagaagagctcggtgtcttggattttgctcttaataaagcgaaaaaaaataaaagtacaaaaaatgaaaatggctggcaacaaaaattgtcagaatctattcaatctgccccgttattttttggcgaagggaatcgtgtacgaaatgaagaagtttataatttgaaggaccttctgttttcagaaatcaagaatgttgaaaatattttaatgagctcggaagcggaagacttaaaaaatataaaaattgaatataaagaagatggcgcgaaaaaagggaactatgtcttgaatgtcttggctagattttacgcgagattcaatgaggatggctatggtggttggaacaaagtaaaaaccgttttggaaaatattgcccgagaggcggggactgatttttcaaaatatggaaataataacaatagaaatgccggcagattttatctaaacggccgcgaacgacaagtttttactctaatcaagtttgaaaaaagtatcacggtggaaaaaatacttgaattggtaaaattacctagcctacttgatgaagcgtatagagatttagtcaacgaaaataaaaatcataaattacgcgacgtaattcaattgagcaagacaattatggctctggttttatctcattctgataaagaaaaacaaattggaggaaattatatccatagtaaattgagcggatacaatgcgcttatttcaaagcgagattttatctcgcggtatagcgtgcaaacgaccaacggaactcaatgtaaattagccataggaaaaggcaaaagcaaaaaaggtaatgaaattgacaggtatttctacgcttttcaattttttaagaatgacgacagcaaaattaatttaaaggtaatcaaaaataattcgcataaaaacatcgatttcaacgacaatgaaaataaaattaacgcattgcaagtgtattcatcaaactatcagattcaattcttagactggttttttgaaaaacatcaagggaagaaaacatcgctcgaggtcggcggatcttttaccatcgccgaaaagagtttgacaatagactggtcggggagtaatccgagagtcggttttaaaagaagcgacacggaagaaaagagggtttttgtctcgcaaccatttacattaataccagacgatgaagacaaagagcgtcgtaaagaaagaatgataaagacgaaaaaccgttttatcggtatcgatatcggtgaatatggtctggcttggagtctaatcgaagtggacaatggagataaaaataatagaggaattagacaacttgagagcggttttattacagacaatcagcagcaagtcttaaagaaaaacgtaaaatcctggaggcaaaaccaaattcgtcaaacgtttacttcaccagacacaaaaattgctcgtcttcgtgaaagtttgatcggaagttacaaaaatcaactggaaagtctgatggttgctaaaaaagcaaatcttagttttgaatacgaagtttccgggtttgaagttgggggaaagagggttgcaaaaatatacgatagtataaagcgtgggtcggtgcgtaaaaaggataataactcacaaaatgatcaaagttggggtaaaaagggaattaatgagtggtcattcgagacgacggctgccggaacatcgcaattttgtactcattgcaagcggtggagcagtttagcgatagtagatattgaagaatatgaattaaaagattacaacgataatttatttaaggtaaaaattaatgatggtgaagttcgtctccttggtaagaaaggttggagatccggcgaaaagatcaaagggaaagaattatttggtcccgtcaaagacgcaatgcgcccaaatgttgacggactagggatgaaaattgtaaaaagaaaatatctaaaacttgatctccgcgattgggtttcaagatatgggaatatggctattttcatctgtccttatgtcgattgccaccatatctctcatgcggataaacaagctgcttttaatattgccgtgcgagggtatttgaaaagcgttaatcctgacagagcaataaaacacggagataaaggtttgtctagggactttttgtgccaagaagagggtaagcttaattttgaacaaatagggttattatgaa

SEQ ID NO: 15 - protein, 1210 aa, Artificial Sequence
  Description of Artificial Sequence Synthetic polypeptide
  CasY.4
MSKRHPRISGVKGYRLHAQRLEYTGKSGAMRTIKYPLYSSPSGGRTVPREIVSAINDDYVGLYGLSNFDDLYNAEKRNEEKVYSVLDFWYDCVQYGAVFSYTAPGLLKNVAEVRGGSYELTKTLKGSHLYDELQIDKVIKFLNKKEISRANGSLDKLKKDIIDCFKAEYRERHKDQCNKLADDIKNAKKDAGASLGERQKKLFRDFFGISEQSENDKPSFTNPLNLTCCLLPFDTVNNNRNRGEVLFNKLKEYAQKLDKNEGSLEMWEYIGIGNSGTAFSNFLGEGFLGRLRENKITELKKAMMDITDAWRGQEQEEELEKRLRILAALTIKLREPKFDNHWGGYRSDINGKLSSWLQNYINQTVKIKEDLKGHKKDLKKAKEMINRFGESDTKEEAVVSSLLESIEKIVPDDSADDEKPDIPAIAIYRRFLSDGRLTLNRFVQREDVQEALIKERLEAEKKKKPKKRKKKSDAEDEKETIDFKELFPHLAKPLKLVPNFYGDSKRELYKKYKNAAIYTDALWKAVEKIYKSAFSSSLKNSFFDTDFDKDFFIKRLQKIFSVYRRFNTDKWKPIVKNSFAPYCDIVSLAENEVLYKPKQSRSRKSAAIDKNRVRLPSTENIAKAGIALARELSVAGFDWKDLLKKEEHEEYIDLIELHKTALALLLAVTETQLDISALDFVENGTVKDFMKTRDGNLVLEGRFLEMFSQSIVFSELRGLAGLMSRKEFITRSAIQTMNGKQAELLYIPHEFQSAKITTPKEMSRAFLDLAPAEFATSLEPESLSEKSLLKLKQMRYYPHYFGYELTRTGQGIDGGVAENALRLEKSPVKKREIKCKQYKTLGRGQNKIVLYVRSSYYQTQFLEWFLHRPKNVQTDVAVSGSFLIDEKKVKTRWNYDALTVALEPVSGSERVFVSQPFTIFPEKSAEEEGQRYLGIDIGEYGIAYTALEITGDSAKILDQNFISDPQLKTLREEVKGLKLDQRRGTFAMPSTKIARIRESLVHSLRNRIHHLALKHKAKIVYELEVSRFEEGKQKIKKVYATLKKADVYSEIDADKNLQTTVWGKLAVASEISASYTSQFCGACKKLWRAEMQVDETITTQELIGTVRVIKGGTLIDAIKDFMRPPIFDENDTPFPKYRDFCDKHHISKKMRGNSCLFICPFCRANADADIQASQTIALLRYVKEEKKVEDYFERFRKLKNIKVLGQMKKI

SEQ ID NO: 16 - DNA, 3636 bp, Artificial Sequence
  Description of Artificial Sequence Synthetic polynucleotide
  CasY.4
atgagtaagcgacatcctagaattagcggcgtaaaagggtaccgtttgcatgcgcaacggctggaatataccggcaaaagtggggcaatgcgaacgattaaatatcctctttattcatctccgagcggtggaagaacggttccgcgcgagatagtttcagcaatcaatgatgattatgtagggctgtacggtttgagtaattttgacgatctgtataatgcggaaaagcgcaacgaagaaaaggtctactcggttttagatttttggtacgactgcgtccaatacggcgcggttttttcgtatacagcgccgggtcttttgaaaaatgttgccgaagttcgcgggggaagctacgaacttacaaaaacgcttaaagggagccatttatatgatgaattgcaaattgataaagtaattaaatttttgaataaaaaagaaatttcgcgagcaaacggatcgcttgataaactgaagaaagacatcattgattgcttcaaagcagaatatcgggaacgacataaagatcaatgcaataaactggctgatgatattaaaaatgcaaaaaaagacgcgggagcttctttaggggagcgtcaaaaaaaattatttcgcgatttttttggaatttcagagcagtctgaaaatgataaaccgtcttttactaatccgctaaacttaacctgctgtttattgccttttgacacagtgaataacaacagaaaccgcggcgaagttttgtttaacaagctcaaggaatatgctcaaaaattggataaaaacgaagggtcgcttgaaatgtgggaatatattggcatcgggaacagcggcactgccttttctaattttttaggagaagggtttttgggcagattgcgcgagaataaaattacagagctgaaaaaagccatgatggatattacagatgcatggcgtgggcaggaacaggaagaagagttagaaaaacgtctgcggatacttgccgcgcttaccataaaattgcgcgagccgaaatttgacaaccactggggagggtatcgcagtgatataaacggcaaattatctagctggcttcagaattacataaatcaaacagtcaaaatcaaagaggacttaaagggacacaaaaaggacctgaaaaaagcgaaagagatgataaataggtttggggaaagcgacacaaaggaagaggcggttgtttcatctttgcttgaaagcattgaaaaaattgttcctgatgatagcgctgatgacgagaaacccgatattccagctattgctatctatcgccgctttctttcggatggacgattaacattgaatcgctttgtccaaagagaagatgtgcaagaggcgctgataaaagaaagattggaagcggagaaaaagaaaaaaccgaaaaagcgaaaaaagaaaagtgacgctgaagatgaaaaagaaacaattgacttcaaggagttatttcctcatcttgccaaaccattaaaattggtgccaaacttttacggcgacagtaagcgtgagctgtacaagaaatataagaacgccgctatttatacagatgctctgtggaaagcagtggaaaaaatatacaaaagcgcgttctcgtcgtctctaaaaaattcattttttgatacagattttgataaagatttttttattaagcggcttcagaaaattttttcggtttatcgtcggtttaatacagacaaatggaaaccgattgtgaaaaactctttcgcgccctattgcgacatcgtctcacttgcggagaatgaagttttgtataaaccgaaacagtcgcgcagtagaaaatctgccgcgattgataaaaacagagtgcgtctcccttccactgaaaatatcgcaaaagctggcattgccctcgcgcgggagctttcagtcgcaggatttgactggaaagatttgttaaaaaaagaggagcatgaagaatacattgatctcatagaattgcacaaaaccgcgcttgcgcttcttcttgccgtaacagaaacacagcttgacataagcgcgttggattttgtagaaaatgggacggtcaaggattttatgaaaacgcgggacggcaatctggttttggaagggcgtttccttgaaatgttctcgcagtcaattgtgttttcagaattgcgcgggcttgcgggtttaatgagccgcaaggaatttatcactcgctccgcgattcaaactatgaacggcaaacaggcggagcttctctacattccgcatgaattccaatcggcaaaaattacaacgccaaaggaaatgagcagggcgtttcttgaccttgcgcccgcggaatttgctacatcgcttgagccagaatcgctttcggagaagtcattattgaaattgaagcagatgcggtactatccgcattattttggatatgagcttacgcgaacaggacaggggattgatggtggagtcgcggaaaatgcgttacgacttgagaagtcgccagtaaaaaaacgagagataaaatgcaaacagtataaaactttgggacgcggacaaaataaaatagtgttatatgtccgcagttcttattatcagacgcaatttttggaatggtttttgcatcggccgaaaaacgttcaaaccgatgttgcggttagcggttcgtttcttatcgacgaaaagaaagtaaaaactcgctggaattatgacgcgcttacagtcgcgcttgaaccagtttccggaagcgagcgggtctttgtctcacagccgtttactatttttccggaaaaaagcgcagaggaagaaggacagaggtatcttggcatagacatcggcgaatacggcattgcgtatactgcgcttgagataactggcgacagtgcaaagattcttgatcaaaattttatttcagacccccagcttaaaactctgcgcgaggaggtcaaaggattaaaacttgaccaaaggcgcgggacatttgccatgccaagcacgaaaatcgcccgcatccgcgaaagccttgtgcatagtttgcggaaccgcatacatcatcttgcgttaaagcacaaagcaaagattgtgtatgaattggaagtgtcgcgttttgaagagggaaagcaaaaaattaagaaagtctacgctacgttaaaaaaagcggatgtgtattcagaaattgacgcggataaaaatttacaaacgacagtatggggaaaattggccgttgcaagcgaaatcagcgcaagctatacaagccagttttgtggtgcgtgtaaaaaattgtggcgggcggaaatgcaggttgacgaaacaattacaacccaagaactaatcggcacagttagagtcataaaagggggcactcttattgacgcgataaaggattttatgcgcccgccgatttttgacgaaaatgacactccatttccaaaatatagagacttttgcgacaagcatcacatttccaaaaaaatgcgtggaaacagctgtttgttcatttgtccattctgccgcgcaaacgcggatgctgatattcaagcaagccaaacaattgcgcttttaaggtatgttaaggaagagaaaaaggtagaggactactttgaacgatttagaaagctaaaaaacattaaagtgctcggacagatgaagaaaatatgatag

SEQ ID NO: 17 - protein, 1192 aa, Artificial Sequence
  Description of Artificial Sequence Synthetic polypeptide
  CasY.5
MAESKQMQCRKCGASMKYEVIGLGKKSCRYMCPDCGNHTSARKIQNKKKRDKKYGSASKAQSQRIAVAGALYPDKKVQTIKTYKYPADLNGEVHDRGVAEKIEQAIQEDEIGLLGPSSEYACWIASQKQSEPYSVVDFWFDAVCAGGVFAYSGARLLSTVLQLSGEESVLRAALASSPFVDDINLAQAEKFLAVSRRTGQDKLGKRIGECFAEGRLEALGIKDRMREFVQAIDVAQTAGQRFAAKLKIFGISQMPEAKQWNNDSGLTVCILPDYYVPEENRADQLVVLLRRLREIAYCMGIEDEAGFEHLGIDPGALSNFSNGNPKRGFLGRLLNNDIIALANNMSAMTPYWEGRKGELIERLAWLKHRAEGLYLKEPHFGNSWADHRSRIFSRIAGWLSGCAGKLKIAKDQISGVRTDLFLLKRLLDAVPQSAPSPDFIASISALDRFLEAAESSQDPAEQVRALYAFHLNAPAVRSIANKAVQRSDSQEWLIKELDAVDHLEFNKAFPFFSDTGKKKKKGANSNGAPSEEEYTETESIQQPEDAEQEVNGQEGNGASKNQKKFQRIPRFFGEGSRSEYRILTEAPQYFDMFCNNMRAIFMQLESQPRKAPRDFKCFLQNRLQKLYKQTFLNARSNKCRALLESVLISWGEFYTYGANEKKFRLRHEASERSSDPDYVVQQALEIARRLFLFGFEWRDCSAGERVDLVEIHKKAISFLLAITQAEVSVGSYNWLGNSTVSRYLSVAGTDTLYGTQLEEFLNATVLSQMRGLAIRLSSQELKDGFDVQLESSCQDNLQHLLVYRASRDLAACKRATCPAELDPKILVLPAGAFIASVMKMIERGDEPLAGAYLRHRPHSFGWQIRVRGVAEVGMDQGTALAFQKPTESEPFKIKPFSAQYGPVLWLNSSSYSQSQYLDGFLSQPKNWSMRVLPQAGSVRVEQRVALIWNLQAGKMRLERSGARAFFMPVPFSFRPSGSGDEAVLAPNRYLGLFPHSGGIEYAVVDVLDSAGFKILERGTIAVNGFSQKRGERQEEAHREKQRRGISDIGRKKPVQAEVDAANELHRKYTDVATRLGCRIVVQWAPQPKPGTAPTAQTVYARAVRTEAPRSGNQEDHARMKSSWGYTWSTYWEKRKPEDILGISTQVYWTGGIGESCPAVAVALLGHIRATSTQTEWEKEEVVFGRLKKFFPS

SEQ ID NO: 18 - DNA, 4560 bp, Artificial Sequence
  Description of Artificial Sequence Synthetic polynucleotide
  CasY.5
accaaccacctattgcgtctttttcgctcattttagcaaaagtggctgtctagacatacaggtggaaaggtgagagtaaagacatggcctgaatagcgtcctcgtcctcgtctagacatacaggtggaaaggtgagagtaaagaccggagcactcatcctctcactctattttgtctagacatacaggtggaaaggtgagagtaaagacaaaccgtgccacactaaaccgatgagtctagacatacaggtggaaaggtgagagtaaagactcaagtaactacctgttctttcacaagtctagacatacaggtggaaaggtgagagtaaagactcaagtaactacctgttctttcacaagtctagacctgcaggtggtaaggtgagagtaaagactcaagtaactacctgttctttcacaagtctagacctgcaggtggtaaggtgagagtaaagacttttatcctcctctctatgcttctgagtctagacatttaggtggaaaggtgagagtaaagacttgtggagatccatgaacttcggcagtctagacctgcaggtggaaaggtgagagtaaagacgtccttcacacgatcttcctctgttagtctaggcctgcaggtggaaaggtgagagtaaagacgcataagcgtaattgaagctctctccggtccagaccttgtcgcgcttgtgttgcgacaaaggcggagtccgcaataagttctttttacaatgttttttccataaaaccgatacaatcaagtatcggttttgctttttttatgaaaatatgttatgctatgtgctcaaataaaaatatcaataaaatagcgtttttttgataatttatcgctaaaattatacataatcacgcaacattgccattctcacacaggagaaaagtcatggcagaaagcaagcagatgcaatgccgcaagtgcggcgcaagcatgaagtatgaagtaattggattgggcaagaagtcatgcagatatatgtgcccagattgcggcaatcacaccagcgcgcgcaagattcagaacaagaaaaagcgcgacaaaaagtatggatccgcaagcaaagcgcagagccagaggatagctgtggctggcgcgctttatccagacaaaaaagtgcagaccataaagacctacaaatacccagcggatctgaatggcgaagttcatgacagaggcgtcgcagagaagattgagcaggcgattcaggaagatgagatcggcctgcttggcccgtccagcgaatacgcttgctggattgcttcacaaaaacaaagcgagccgtattcagttgtagatttttggtttgacgcggtgtgcgcaggcggagtattcgcgtattctggcgcgcgcctgctttccacagtcctccagttgagtggcgaggaaagcgttttgcgcgctgctttagcatctagcccgtttgtagatgacattaatttggcgcaagcggaaaagttcctagccgttagccggcgcacaggccaagataagctaggcaagcgcattggagaatgtttcgcggaaggccggcttgaagcgcttggcatcaaagatcgcatgcgcgaattcgtgcaagcgattgatgtggcccaaaccgcgggccagcggttcgcggccaagctaaagatattcggcatcagtcagatgcctgaagccaagcaatggaacaatgattccgggctcactgtatgtattttgccggattattatgtcccggaagaaaaccgcgcggaccagctggttgttttgcttcggcgcttacgcgagatcgcgtattgcatgggaattgaggatgaagcaggatttgagcatctaggcattgaccctggcgctctttccaatttttccaatggcaatccaaagcgaggatttctcggccgcctgctcaataatgacattatagcgctggcaaacaacatgtcagccatgacgccgtattgggaaggcagaaaaggcgagttgattgagcgccttgcatggcttaaacatcgcgctgaaggattgtatttgaaagagccacatttcggcaactcctgggcagaccaccgcagcaggattttcagtcgcattgcgggctggctttccggatgcgcgggcaagctcaagattgccaaggatcagatttcaggcgtgcgtacggatttgtttctgctcaagcgccttctggatgcggtaccgcaaagcgcgccgtcgccggactttattgcttccatcagcgcgctggatcggtttttggaagcggcagaaagcagccaggatccggcagaacaggtacgcgctttgtacgcgtttcatctgaacgcgcctgcggtccgatccatcgccaacaaggcggtacagaggtctgattcccaggagtggcttatcaaggaactggatgctgtagatcaccttgaattcaacaaagcatttccgtttttttcggatacaggaaagaaaaagaagaaaggagcgaatagcaacggagcgccttctgaagaagaatacacggaaacagaatccattcaacaaccagaagatgcagagcaggaagtgaatggtcaagaaggaaatggcgcttcaaagaaccagaaaaagtttcagcgcattcctcgatttttcggggaagggtcaaggagtgagtatcgaattttaacagaagcgccgcaatattttgacatgttctgcaataatatgcgcgcgatctttatgcagctagagagtcagccgcgcaaggcgcctcgtgatttcaaatgctttctgcagaatcgtttgcagaagctttacaagcaaacctttctcaatgctcgcagtaataaatgccgcgcgcttctggaatccgtccttatttcatggggagaattttatacttatggcgcgaatgaaaagaagtttcgtctgcgccatgaagcgagcgagcgcagctcggatccggactatgtggttcagcaggcattggaaatcgcgcgccggcttttcttgttcggatttgagtggcgcgattgctctgctggagagcgcgtggatttggttgaaatccacaaaaaagcaatctcatttttgcttgcaatcactcaggccgaggtttcagttggttcctataactggcttgggaatagcaccgtgagccggtatctttcggttgctggcacagacacattgtacggcactcaactggaggagtttttgaacgccacagtgctttcacagatgcgtgggctggcgattcggctttcatctcaggagttaaaagacggatttgatgttcagttggagagttcgtgccaggacaatctccagcatctgctggtgtatcgcgcttcgcgcgacttggctgcgtgcaaacgcgctacatgcccggctgaattggatccgaaaattcttgttctgccggctggtgcgtttatcgcgagcgtaatgaaaatgattgagcgtggcgatgaaccattagcaggcgcgtatttgcgtcatcggccgcattcattcggctggcagatacgggttcgtggagtggcggaagtaggcatggatcagggcacagcgctagcattccagaagccgactgaatcagagccgtttaaaataaagccgttttccgctcaatacggcccagtactttggcttaattcttcatcctatagccagagccagtatctggatggatttttaagccagccaaagaattggtctatgcgggtgctacctcaagccggatcagtgcgcgtggaacagcgcgttgctctgatatggaatttgcaggcaggcaagatgcggctggagcgctctggagcgcgcgcgtttttcatgccagtgccattcagcttcaggccgtctggttcaggagatgaagcagtattggcgccgaatcggtacttgggactttttccgcattccggaggaatagaatacgcggtggtggatgtattagattccgcgggtttcaaaattcttgagcgcggtacgattgcggtaaatggcttttcccagaagcgcggcgaacgccaagaggaggcacacagagaaaaacagagacgcggaatttctgatataggccgcaagaagccggtgcaagctgaagttgacgcagccaatgaattgcaccgcaaatacaccgatgttgccactcgtttagggtgcagaattgtggttcagtgggcgccccagccaaagccgggcacagcgccgaccgcgcaaacagtatacgcgcgcgcagtgcggaccgaagcgccgcgatctggaaatcaagaggatcatgctcgtatgaaatcctcttggggatatacctggagcacctattgggagaagcgcaaaccagaggatattttgggcatctcaacccaagtatactggaccggcggtataggcgagtcatgtcccgcagtcgcggttgcgcttttggggcacattagggcaacatccactcaaactgaatgggaaaaagaggaggttgtattcggtcgactgaagaagttctttccaagctagacgatctttttaaaaactgggctgctggctatcgtatggtcagtagctcttatttttttacttgatatatggtattat

SEQ ID NO: 19 - protein, 1287 aa, Artificial Sequence
  Description of Artificial Sequence Synthetic polypeptide
  CasY.6
MKRILNSLKVAALRLLFRGKGSELVKTVKYPLVSPVQGAVEELAEAIRHDNLHLFGQKEIVDLMEKDEGTQVYSVVDFWLDTLRLGMFFSPSANALKITLGKFNSDQVSPFRKVLEQSPFFLAGRLKVEPAERILSVEIRKIGKRENRVENYAADVETCFIGQLSSDEKQSIQKLANDIWDSKDHEEQRMLKADFFAIPLIKDPKAVTEEDPENETAGKQKPLELCVCLVPELYTRGFGSIADFLVQRLTLLRDKMSTDTAEDCLEYVGIEEEKGNGMNSLLGTFLKNLQGDGFEQIFQFMLGSYVGWQGKEDVLRERLDLLAEKVKRLPKPKFAGEWSGHRMFLHGQLKSWSSNFFRLFNETRELLESIKSDIQHATMLISYVEEKGGYHPQLLSQYRKLMEQLPALRTKVLDPEIEMTHMSEAVRSYIMIHKSVAGFLPDLLESLDRDKDREFLLSIFPRIPKIDKKTKEIVAWELPGEPEEGYLFTANNLFRNFLENPKHVPRFMAERIPEDWTRLRSAPVWFDGMVKQWQKVVNQLVESPGALYQFNESFLRQRLQAMLTVYKRDLQTEKFLKLLADVCRPLVDFFGLGGNDIIFKSCQDPRKQWQTVIPLSVPADVYTACEGLAIRLRETLGFEWKNLKGHEREDFLRLHQLLGNLLFWIRDAKLVVKLEDWMNNPCVQEYVEARKAIDLPLEIFGFEVPIFLNGYLFSELRQLELLLRRKSVMTSYSVKTTGSPNRLFQLVYLPLNPSDPEKKNSNNFQERLDTPTGLSRRFLDLTLDAFAGKLLTDPVTQELKTMAGFYDHLFGFKLPCKLAAMSNHPGSSSKMVVLAKPKKGVASNIGFEPIPDPAHPVFRVRSSWPELKYLEGLLYLPEDTPLTIELAETSVSCQSVSSVAFDLKNLTTILGRVGEFRVTADQPFKLTPIIPEKEESFIGKTYLGLDAGERSGVGFAIVTVDGDGYEVQRLGVHEDTQLMALQQVASKSLKEPVFQPLRKGTFRQQERIRKSLRGCYWNFYHALMIKYRAKVVHEESVGSSGLVGQWLRAFQKDLKKADVLPKKGGKNGVDKKKRESSAQDTLWGGAFSKKEEQQIAFEVQAAGSSQFCLKCGWWFQLGMREVNRVQESGVVLDWNRSIVTFLIESSGEKVYGFSPQQLEKGFRPDIETFKKMVRDFMRPPMFDRKGRPAAAYERFVLGRRHRRYRFDKVFEERFGRSALFICPRVGCGNFDHSSEQSAVVLALIGYIADKEGMSGKKLVYVRLAELMAEWKLKKLERSRVEEQSSAQ

SEQ ID NO: 20 - DNA, 3864 bp, Artificial Sequence
  Description of Artificial Sequence Synthetic polynucleotide
  CasY.6
atgaagagaattctgaacagtctgaaagttgctgccttgagacttctgtttcgaggcaaaggttctgaattagtgaagacagtcaaatatccattggtttccccggttcaaggcgcggttgaagaacttgctgaagcaattcggcacgacaacctgcacctttttgggcagaaggaaatagtggatcttatggagaaagacgaaggaacccaggtgtattcggttgtggatttttggttggataccctgcgtttagggatgtttttctcaccatcagcgaatgcgttgaaaatcacgctgggaaaattcaattctgatcaggtttcaccttttcgtaaggttttggagcagtcacctttttttcttgcgggtcgcttgaaggttgaacctgcggaaaggatactttctgttgaaatcagaaagattggtaaaagagaaaacagagttgagaactatgccgccgatgtggagacatgcttcattggtcagctttcttcagatgagaaacagagtatccagaagctggcaaatgatatctgggatagcaaggatcatgaggaacagagaatgttgaaggcggatttttttgctatacctcttataaaagaccccaaagctgtcacagaagaagatcctgaaaatgaaacggcgggaaaacagaaaccgcttgaattatgtgtttgtcttgttcctgagttgtatacccgaggtttcggctccattgctgattttctggttcagcgacttaccttgctgcgtgacaaaatgagtaccgacacggcggaagattgcctcgagtatgttggcattgaggaagaaaaaggcaatggaatgaattccttgctcggcacttttttgaagaacctgcagggtgatggttttgaacagatttttcagtttatgcttgggtcttatgttggctggcaggggaaggaagatgtactgcgcgaacgattggatttgctggccgaaaaagtcaaaagattaccaaagccaaaatttgccggagaatggagtggtcatcgtatgtttctccatggtcagctgaaaagctggtcgtcgaatttcttccgtctttttaatgagacgcgggaacttctggaaagtatcaagagtgatattcaacatgccaccatgctcattagctatgtggaagagaaaggaggctatcatccacagctgttgagtcagtatcggaagttaatggaacaattaccggcgttgcggactaaggttttggatcctgagattgagatgacgcatatgtccgaggctgttcgaagttacattatgatacacaagtctgtagcgggatttctgccggatttactcgagtctttggatcgagataaggatagggaatttttgctttccatctttcctcgtattccaaagatagataagaagacgaaagagatcgttgcatgggagctaccgggcgagccagaggaaggctatttgttcacagcaaacaaccttttccggaattttcttgagaatccgaaacatgtgccacgatttatggcagagaggattcccgaggattggacgcgtttgcgctcggcccctgtgtggtttgatgggatggtgaagcaatggcagaaggtggtgaatcagttggttgaatctccaggcgccctttatcagttcaatgaaagttttttgcgtcaaagactgcaagcaatgcttacggtctataagcgggatctccagactgagaagtttctgaagctgctggctgatgtctgtcgtccactcgttgattttttcggacttggaggaaatgatattatcttcaagtcatgtcaggatccaagaaagcaatggcagactgttattccactcagtgtcccagcggatgtttatacagcatgtgaaggcttggctattcgtctccgcgaaactcttggattcgaatggaaaaatctgaaaggacacgagcgggaagattttttacggctgcatcagttgctgggaaatctgctgttctggatcagggatgcgaaacttgtcgtgaagctggaagactggatgaacaatccttgtgttcaggagtatgtggaagcacgaaaagccattgatcttcccttggagattttcggatttgaggtgccgatttttctcaatggctatctcttttcggaactgcgccagctggaattgttgctgaggcgtaagtcggtgatgacgtcttacagcgtcaaaacgacaggctcgccaaataggctcttccagttggtttacctacctctaaacccttcagatccggaaaagaaaaattccaacaactttcaggagcgcctcgatacacctaccggtttgtcgcgtcgttttctggatcttacgctggatgcatttgctggcaaactcttgacggatccggtaactcaggaactgaagacgatggccggtttttacgatcatctctttggcttcaagttgccgtgtaaactggcggcgatgagtaaccatccaggatcctcttccaaaatggtggttctggcaaaaccaaagaagggtgttgctagtaacatcggctttgaacctattcccgatcctgctcatcctgtgttccgggtgagaagttcctggccggagttgaagtacctggaggggttgttgtatcttcccgaagatacaccactgaccattgaactggcggaaacgtcggtcagttgtcagtctgtgagttcagtcgctttcgatttgaagaatctgacgactatcttgggtcgtgttggtgaattcagggtgacggcagatcaacctttcaagctgacgcccattattcctgagaaagaggaatccttcatcgggaagacctacctcggtcttgatgctggagagcgatctggcgttggtttcgcgattgtgacggttgacggcgatgggtatgaggtgcagaggttgggtgtgcatgaagatactcagcttatggcgcttcagcaagtcgccagcaagtctcttaaggagccggttttccagccactccgtaagggcacatttcgtcagcaggagcgcattcgcaaaagcctccgcggttgctactggaatttctatcatgcattgatgatcaagtaccgagctaaagttgtgcatgaggaatcggtgggttcatccggtctggtggggcagtggctgcgtgcatttcagaaggatctcaaaaaggctgatgttctgcccaagaagggtggaaaaaatggtgtagacaaaaaaaagagagaaagcagcgctcaggataccttatggggaggagctttctcgaagaaggaagagcagcagatagcctttgaggttcaggcagctggatcaagccagttttgtctgaagtgtggttggtggtttcagttggggatgcgggaagtaaatcgtgtgcaggagagtggcgtggtgctggactggaaccggtccattgtaaccttcctcatcgaatcctcaggagaaaaggtatatggtttcagtcctcagcaactggaaaaaggctttcgtcctgacatcgaaacgttcaaaaaaatggtaagggattttatgagaccccccatgtttgatcgcaaaggtcggccggccgcggcgtatgaaagattcgtactgggacgtcgtcaccgtcgttatcgctttgataaagtttttgaagagagatttggtcgcagtgctcttttcatctgcccgcgggtcgggtgtgggaatttcgatcactccagtgagcagtcagccgttgtccttgcccttattggttacattgctgataaggaagggatgagtggtaagaagcttgtttatgtgaggctggctgaacttatggctgagtggaagctgaagaaactggagagatcaagggtggaagaacagagctcggcacaataa

(19) United States
(12) Patent Application Publication
(10) Pub. No.: US 2022/0290177 A1
(43) Pub. Date: Sep. 15, 2022

(54) COMPOSITIONS AND METHODS FOR EXCISION WITH SINGLE GRNA

(71) Applicant: Excision BioTherapeutics Inc, Philadelphia, PA (US)

(72) Inventor: Thomas MALCOLM, Andover, NJ (US)

(21) Appl. No.: 17/200,574

(22) Filed: Mar. 12, 2021

Related U.S. Application Data
(63) Continuation of application No. PCT/US2019/050507, filed on Sep. 11, 2019.
(60) Provisional application No. 62/730,901, filed on Sep. 13, 2018.

(51) Int. Cl. C12N 15/86; C12N 9/22; C12N 15/11; A61K 31/7125; A61K 31/7105; A61K 38/46

(52) U.S. Cl. CPC C12N 15/86; C12N 9/22; C12N 15/11; A61K 31/7125; A61K 31/7105; A61K 38/465; C12N 2310/20; C12N 2800/80; C12N 2750/14143

(57) ABSTRACT

A method of excising undesired DNA or RNA from cells, by administering a composition including a vector encoding at least one gene editor and at least one gRNA to an individual, and excising the DNA or RNA from cells, wherein cut repair is made by microhomology-mediated end joining (MMEJ).

### CROSS-REFERENCES

This application is a continuation of International Application No. PCT/US2019/050507, filed Sep. 11, 2019, which claims the benefit of U.S. Provisional Patent Application No. 62/730,901, filed Sep. 13, 2018, each of which is incorporated by reference herein in its entirety.

### SEQUENCE LISTINGS

This application incorporates by reference a Sequence Listing submitted with this application as text file entitled 56852-733_301_SL.txt, created on Jan. 20, 2022 and having a size of 146,390 bytes.

### BACKGROUND OF THE INVENTION

### 1. Technical Field

The present invention relates methods of excision of DNA and RNA. More specifically, the present invention relates to compositions and treatments for excising viruses or cancer from infected cells and inactivating viruses within the cells.

### 2. Background Art

Gene editing allows DNA or RNA to be inserted, deleted, or replaced in an organism's genome by the use of nucleases. There are several types of nucleases currently used, including meganucleases, zinc finger nucleases, transcription activator-like effector-based nucleases (TALENs), and clustered regularly interspaced short palindromic repeats (CRISPR)-Cas nucleases. These nucleases can create site-specific double strand breaks of the DNA in order to edit the DNA.

Meganucleases have very long recognition sequences and are very specific to DNA. While meganucleases are less toxic than other gene editors, they are expensive to construct, as not many are known and mutagenesis must be used to create variants that recognize specific sequences.

Both zinc-finger and TALEN nucleases are non-specific for DNA but can be linked to DNA sequence recognizing peptides. However, each of these nucleases can produce off-target effects and cytotoxicity and require time to create the DNA sequence recognizing peptides.

CRISPR-Cas nucleases are derived from prokaryotic systems and can use the Cas9 nuclease, the Cpf1 nuclease, or other Cas nucleases for DNA editing. CRISPR is an adaptive immune system found in many microbial organisms. While the CRISPR system was not well understood, it was found that there were genes associated to the CRISPR regions that coded for exonucleases and/or helicases, called CRISPR-associated proteins (Cas). Several different types of Cas proteins were found, some using multi-protein complexes (Type I), some using singe effector proteins with a universal tracrRNA and crRNA specific for a target DNA sequence (Type II), and some found in archea (Type III). Cas9 (a Type II Cas protein) was discovered when the bacteria Streptococcus thermophilus was being studied and an unusual CRISPR locus was found (Bolotin, et al. 2005). It was also found that the spacers share a common sequence at one end (the protospacer adjacent motif PAM) and is used for target sequence recognition. Cas9 was not found with a screen but by examining a specific bacteria.

U.S. patent application Ser. No. 14/838,057 to Khalili, et al. discloses a method of inactivating a proviral DNA integrated into the genome of a host cell latently infected with a retrovirus, by treating the host cell with a composition comprising a Clustered Regularly Interspaced Short Palindromic Repeat (CRISPR)-associated endonuclease, and two or more different guide RNAs (gRNAs), wherein each of the at least two gRNAs is complementary to a different target nucleic acid sequence in a long terminal repeat (LTR) of the proviral DNA; and inactivating the proviral DNA. A composition is also provided for inactivating proviral DNA. Delivery of the CRISPR-associated endonuclease and gRNAs can be by various expression vectors, such as plasmid vectors, lentiviral vectors, adenoviral vectors, or adeno-associated virus vectors.

Viruses replicate by one of two cycles, either the lytic cycle or the lysogenic cycle. In the lytic cycle, first the virus penetrates a host cell and releases its own nucleic acid. Next, the host cell's metabolic machinery is used to replicate the viral nucleic acid and accumulate the virus within the host cell. Once enough virions are produced within the host cell, the host cell bursts (lysis) and the virions go on to infect additional cells. Lytic viruses can integrate viral DNA into the host genome as well as be non-integrated where lysis does not occur over the period of the infection of the cell.

Lytic viruses include John Cunningham virus (JCV), hepatitis A, hepatitis C, and various herpesviruses. In the lysogenic cycle, virion DNA is integrated into the host cell, and when the host cell reproduces, the virion DNA is copied into the resulting cells from cell division. In the lysogenic cycle, the host cell does not burst. Lysogenic viruses include hepatitis B, Zika virus, and HIV.

While the methods and compositions described above are useful in treating lysogenic viruses that have been integrated into the genome of a host cell, gene editing systems are not able to effectively treat lytic viruses. Treating a lytic virus will result in inefficient clearance of the virus if solely using this system unless inhibitor drugs are available to suppress viral expression, as in the case of HIV. Most viruses presently lack targeted inhibitor drugs. In particular, the CRISPR-associated nuclease cannot access viral nucleic acid that is contained within the virion (that is, protected by capsid or envelope proteins for example).

Researchers from the Broad Institute of MIT and Harvard, Mass. Institute of Technology, the National Institutes of Health, Rutgers University New Brunswick and the Skolkovo Institute of Science and Technology have characterized a new CRISPR system that targets RNA, rather than DNA. This approach has the potential to open an additional avenue in cellular manipulation relating to editing RNA. Whereas DNA editing makes permanent changes to the genome of a cell, the CRISPR-based RNA-targeting approach can allow temporary changes that can be adjusted up or down, and with greater specificity and functionality than existing methods for RNA interference. Specifically, it can address RNA embedded viral infections and resulting disease. The study reports the identification and functional characterization of C2c2, an RNA-guided enzyme capable of targeting and degrading RNA.

The findings reveal that C2c2—the first naturally-occurring CRISPR system that targets only RNA to have been identified, discovered by this collaborative group in October 2015—helps protect bacteria against viral infection. They demonstrate that C2c2 can be programmed to cleave particular RNA sequences in bacterial cells, which would make it an important addition to the molecular biology toolbox. The RNA-focused action of C2c2 complements the CRISPR-Cas9 system, which targets DNA, the genomic blueprint for cellular identity and function. The ability to target only RNA, which helps carry out the genomic instructions, offers the ability to specifically manipulate RNA in a high-throughput manner—and manipulate gene function more broadly. This has the potential to accelerate progress to understand, treat and prevent disease. Other compositions can be used to target RNA, such as siRNA/miRNA/shRNA/RNAi which do not use a nuclease-based mechanism, and therefore one or more are utilized for the degradative silencing on viral RNA transcripts (non-coding or coding).

When a CRISPR enzyme makes a cut in a DNA strand (a double strand break), the resulting cut is repaired by a general repair pathway, usually the non-homologous end joining (NHEJ) mechanism. Until now, the use of single gRNAs to deactivate viral genomes has been plagued with the insertion of random base pairs or deletions through the NHEJ mechanism. This results in a population of virus that is indeed deactivated, but in a high number of cases, the NHEJ will lead to base insertions that allow for viral escape and even in some cases viruses that become hyper-activated. For example, Wang, et al. (Molecular Therapy, Vol. 24, Issue 3, March 2016) shows that CRISPR-Cas9 can be used for cleavage of HIV proviral DNA in infected cells with treatment of Cas9 and an anti-HIV gRNA, but the virus rapidly and consistently escaped inhibition due to nucleotide insertions, deletions, and substitutions due to NHEJ DNA repair.

Ata, et al. (Plos Genet 2018 Sep. 12; 14(9):e1007652) use a method of microhomology-mediated end joining (MMEJ) as a viable solution for improving somatic sequence homogeneity in vivo, capable of generating a single predictable allele at high rates (56%˜86% of the entire mutant allele pool). Ata, et al. found that whereas somatic mosaicism hinders efficient recreation of knockout mutant allele at base pair resolution via the standard NHEJ-based approach, FO founders transmitted the identical MMEJ allele of interest at high rates.

There remains a need for compositions and methods of excising viruses and cancers using single gRNAs that do not induce cells that escape inhibition.

### SUMMARY OF THE INVENTION

The present invention provides for a method of excising undesired DNA or RNA from cells, by administering a composition including a vector encoding at least one gene editor and at least one gRNA to an individual, and excising the undesired DNA or RNA from cells, wherein cut repair is made by microhomology-mediated end joining (MMEJ).

### DESCRIPTION OF THE DRAWINGS

Other advantages of the present invention are readily appreciated as the same becomes better understood by reference to the following detailed description when considered in connection with the accompanying drawings wherein:

FIG. 1 is a picture of lytic and lysogenic virus within a cell and at which point CRISPR Cas9 can be used and at which point RNA targeting systems can be used; and

FIG. 2 is a chart of various Archaea Cas9 effectors, CasY.1-CasY.6 effectors, and CasX effectors of the present invention.

### DETAILED DESCRIPTION OF THE INVENTION

The present invention is generally directed to methods for treating lysogenic and lytic viruses as well as cancer in cells with various gene editing systems and enzyme effectors with at least one gRNA with cut repair by MMEJ. The compositions can treat both lysogenic viruses and lytic viruses, or optionally viruses that use both methods of replication.

The term “vector” includes cloning and expression vectors, as well as viral vectors and integrating vectors. An “expression vector” is a vector that includes a regulatory region. Vectors are also further described below.

The term “lentiviral vector” includes both integrating and non-integrating lentiviral vectors.

Viruses replicate by one of two cycles, either the lytic cycle or the lysogenic cycle. In the lytic cycle, first the virus penetrates a host cell and releases its own nucleic acid. Next, the host cell's metabolic machinery is used to replicate the viral nucleic acid and accumulate the virus within the host cell. Once enough virions are produced within the host cell, the host cell bursts (lysis) and the virions go on to infect additional cells. Lytic viruses can integrate viral DNA into the host genome as well as be non-integrated where lysis does not occur over the period of the infection of the cell. Viruses such as lambda phage can switch between lytic and lysogenic cycles.

“Lysogenic virus” as used herein, refers to a virus that replicates by the lysogenic cycle (i.e. does not cause the host cell to burst and integrates viral nucleic acid into the host cell DNA). The lysogenic virus can mainly replicate by the lysogenic cycle but sometimes replicate by the lytic cycle. In the lysogenic cycle, virion DNA is integrated into the host cell, and when the host cell reproduces, the virion DNA is copied into the resulting cells from cell division. In the lysogenic cycle, the host cell does not burst.

“Lytic virus” as used herein refers to a virus that replicates by the lytic cycle (i.e. causes the host cell to burst after an accumulation of virus within the cell). The lytic virus can mainly replicate by the lytic cycle but sometimes replicate by the lysogenic cycle.

“gRNA” as used herein refers to guide RNA. The gRNAs in the CRISPR Cas9 systems and other CRISPR nucleases herein are used for the excision of viral genome segments and hence the crippling disruption of the virus' capability to replicate/produce protein. This is accomplished by using two or more specifically designed gRNAs to avoid the issues seen with single gRNAs such as viral escape or mutations. The gRNA can be a sequence complimentary to a coding or a non-coding sequence and can be tailored to the particular virus to be targeted. The gRNA can be a sequence complimentary to a protein coding sequence, for example, a sequence encoding one or more viral structural proteins, (e.g., gag, pol, env and tat). The gRNA sequence can be a sense or anti-sense sequence. It should be understood that when a gene editor composition is administered herein, preferably this includes two or more gRNA.

“Nucleic acid” as used herein, refers to both RNA and DNA, including cDNA, genomic DNA, synthetic DNA, and DNA (or RNA) containing nucleic acid analogs, any of which may encode a polypeptide of the invention and all of which are encompassed by the invention. Polynucleotides can have essentially any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, short hairpin RNA (shRNA), interfering RNA (RNAi), ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. In the context of the present invention, nucleic acids can encode a fragment of a naturally occurring Cas9 or a biologically active variant thereof and at least two gRNAs where in the gRNAs are complementary to a sequence in a virus.

An “isolated” nucleic acid can be, for example, a naturally-occurring DNA molecule or a fragment thereof, provided that at least one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among many (e.g., dozens, or hundreds to millions) of other nucleic acids within, for example, cDNA libraries or genomic libraries, or gel slices containing a genomic DNA restriction digest, is not an isolated nucleic acid.

Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring portion of a Cas9-encoding DNA (in accordance with, for example, the formula above).

The present invention provides for a method of excising undesired DNA or RNA from cells, by administering a composition including a vector encoding at least one gene editor and at least one gRNA to an individual and excising the undesired DNA or RNA from cells.

A single gRNA can be used along with the MMEJ method described above of deactivation (single use) or multiple gRNAs can be used with the MMEJ method for excision of genomes. Much of the current work in this area has been focused on trying to get single gRNAs to work in the context of viruses, by 1) targeting of structured regions (such as TAR in HIV-Cullen paper), by 2) changing the structure of Cas9 to eliminate NHEJ (which has not been fruitful), or by 3) attaching deaminase (or deaminase-like) domains to dCas9 to make basepair substitutions. Each of these current methods have issues. In method 1, targeting of structured portions of viral genomes such as TAR may reduce viral escape, but HIV can still activate through the NFkB pathway, therefore two or more gRNAs would have to be used. Other viruses may have similar issues. In method 2, Cas9 engineering in this case is labor intensive and it is unlikely results will be obtainable soon. In method 3, adding another domain to make point mutations without cutting, is really no better than excising and there will be delivery issues due to the size of the deaminase/dcas9 complex. The present invention solves these issues by using the MMEJ method with single or multiple gRNAs to prevent off-target effects.

In MMEJ, 5-25 base pair microhomologous sequences are used to align the broken strands of DNA before joining. MMEJ uses a Ku protein and DNA-PK independent repair mechanism, and repair occurs during the S-phase of the cell cycle, as opposed to the G0/G1 and early S-phases in NHEJ and late S to G2-phase for homologous recombinational repair (HRR). MMEJ works by ligating the mismatched hanging strands of DNA, removing overhanging nucleotides, and filling in the missing base pairs. When a break occurs, a homology of 5-25 complementary base pairs on both strands is identified and used as a basis for which to align the strands with mismatched ends. Once aligned, any overhanging bases (flaps) and mismatched bases on the strands are removed and any missing nucleotides are inserted.

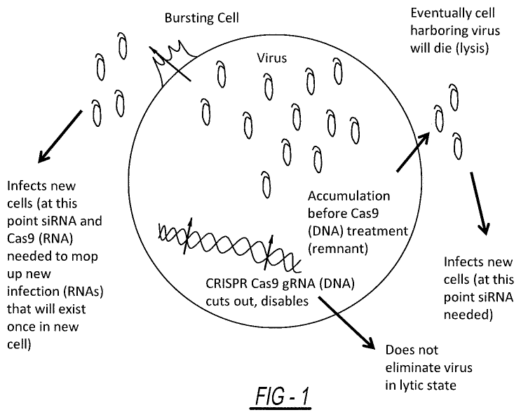

Preferably, the composition includes isolated nucleic acid encoding a CRISPR-associated endonuclease (such as Cas9 or any other gene editor described below) and one or more gRNAs that are complementary to a target sequence in a virus or cancer. Each gRNA can be complimentary to a different sequence within the virus. The composition removes the replication critical segment of the viral genome (DNA) (or RNA using RNA editors such as C2c2) within the genome itself and translation products using RNA editors such as C2c2. When lytic viruses are targeted, the composition can also include small interfering RNA (siRNA)/microRNA (miRNA), short hairpin RNA, and interfering RNA (RNAi) (for RNA interference) that target critical RNAs (viral mRNA) that translate (non-coding or coding) viral proteins involved with the formation of viral proteins and/or virions. As shown in FIG. 1, lytic and lysogenic viruses need to be treated in different ways. While CRISPR Cas9 is usually used to target DNA, this gene editing system can be designed to target RNA within the virus instead in order to target lytic viruses. For example, Nelles, et al. (Cell, Volume 165, Issue 2, p. 488-496, Apr. 7, 2016) shows that RNA-targeting Cas9 was able to bind mRNAs.

Most preferably, an entire viral genome can be excised from the host cell infected with virus. Viral or cancer DNA or RNA can be excised, depending on the type of virus. Alternatively, additions, deletions, or mutations can be made in the genome of the virus. The composition can optionally include other CRISPR or gene editing systems that target DNA. The gRNAs are designed to be the most optimal in safety to provide no off-target effects and no viral escape. The composition can treat any virus in the tables below that are indicated as having a lysogenic replication cycle, lytic replication cycle, or both and is especially useful for retroviruses. The composition can be delivered by a vector or any other method as described below.

The undesired DNA or RNA can also be in any cancer cell or pre-cancerous cell, especially virus-induced cancer. The cancer cells targeted can be associated with adenoid cystic carcinoma, adrenal gland tumors, amyloidosis, anal cancer, appendix cancer, astrocytoma, ataxia-telangiectasia, attenuated familial adenomatous polyposis, Beckwith-Wiedermann Syndrome, bile duct cancer, Birt-Hogg-Dube Syndrome, bladder cancer, bone cancer, brain stem glioma, brain tumors, breast cancer, carcinoid tumors, Carney complex, central nervous system tumors, cervical cancer, colorectal cancer, Cowden syndrome, craniopharyngioma, desmoplastic infantile ganglioglioma, endocrine tumors, ependymoma, esophageal cancer, Ewing sarcoma, eye cancer, eyelid cancer, fallopian tube cancer, familial adenomatous polyposis, familial malignant melanoma, familial non-VHL clear cell renal cell carcinoma, gallbladder cancer, Gardner Syndrome, gastrointestinal stromal tumor, germ cell tumor, gestational trophoblastic disease, head and neck cancer, diffuse gastric cancer, leiomyomatosis and renal cell cancer, mixed polyposis syndrome, pancreatitis, papillary renal cell carcinoma, HIV and AIDS-related cancer, islet cell tumors, juvenile polyposis syndrome, kidney cancer, lacrimal gland tumor, laryngeal and hypopharyngeal cancer, acute lymphoblastic leukemia, acute lymphocytic leukemia, acute myeloid leukemia, B-cell prolymphocytic leukemia, hairy cell leukemia, chronic lymphocytic leukemia, chronic myeloid leukemia, chronic T-cell lymphocytic leukemia, eosinophilic leukemia, Li-Fraumeni Syndrome, liver cancer, lung cancer, Hodgkin lymphoma, Non-Hodgkin lymphoma, Lynch Syndrome, mastocytosis, medulloblastoma, melanoma, meningioma, mesothelioma, Muir-Torre Syndrome, multiple endocrine neoplasia type 1, multiple endocrine neoplasia type 2, multiple myeloma, myelodysplastic syndromes, MYH-associated polyposis, nasal cavity and paranasal sinus cancer, nasopharyngeal cancer, neuroblastoma, neuroendocrine tumors, neurofibromatosis type 1, neurofibromatosis type 2, nevoid basal cell carcinoma syndrome, oral and oropharyngeal cancer, osteosarcoma, ovarian cancer, pancreatic cancer, parathyroid cancer, penile cancer, Peutz-Jeghers Syndrome, pituitary gland tumors, pleuropulmonary blastoma, prostate cancer, retinoblastoma, rhabdomyosarcoma, salivary gland cancer, sarcoma, alveolar soft part and cardiac sarcoma, Kaposi sarcoma, skin cancer, small bowel cancer, stomach cancer, testicular cancer, thymoma, thyroid cancer, tuberous sclerosis syndrome, Turcot Syndrome, unknown primary, uterine cancer, vaginal cancer, Von Hippel-Lindau Syndrome, Wilms tumors, or Xeroderma pigmentosum.

There are many different gene editors (CRISPR systems or others) and enzyme effectors that can be used with the methods and compositions of the present invention to target either DNA or RNA in viruses. These include Argonaute proteins, RNase P RNA, C2c1, C2c2, C2c3, various Cas9 enzymes, Cpf1, TevCas9, Archaea Cas9, CasY.1-CasY.6 effectors, and CasX effectors. Any other composition that targets RNA such as siRNA/miRNA/shRNAs/RNAi can also be used. Each of these are further described below.

“Argonaute protein” as used herein, refers to proteins of the PIWI protein superfamily that contain a PIWI (P element-induced wimpy testis) domain, a MID (middle) domain, a PAZ (Piwi-Argonaute-Zwille) domain and an N-terminal domain. Argonaute proteins are capable of binding small RNAs, such as microRNAs, small interfering RNAs (siRNAs), and Piwi-interacting RNAs. Argonaute proteins can be guided to target sequences with these RNAs in order to cleave mRNA, inhibit translation, or induce mRNA degradation in the target sequence. There are several different human Argonaute proteins, including AGO1, AGO2, AGO3, and AGO4 that associate with small RNAs. AGO2 has slicer ability, i.e. acts as an endonuclease. Argonaute proteins can be used for gene editing. Endonucleases from the Argonaute protein family (from Natronobacterium gregoryi Argonaute) also use oligonucleotides as guides to degrade invasive genomes. Work by Gao et al has shown that the Natronobacterium gregoryi Argonaute (NgAgo) is a DNA-guided endonuclease suitable for genome editing in human cells. NgAgo binds 5′ phosphorylated single-stranded guide DNA (gDNA) of ˜24 nucleotides, efficiently creates site-specific DNA double-strand breaks when loaded with the gDNA. The NgAgo-gDNA system does not require a protospacer-adjacent motif (PAM), as does Cas9, and preliminary characterization suggests a low tolerance to guide-target mismatches and high efficiency in editing (G+C)-rich genomic targets. The Argonaute protein endonucleases used in the present invention can also be Rhodobacter sphaeroides Argonaute (RsArgo). RsArgo can provide stable interaction with target DNA strands and guide RNA, as it is able to maintain base-pairing in the 3′-region of the guide RNA between the N-terminal and PIWI domains. RsArgo is also able to specifically recognize the 5′ base-U of guide RNA, and the duplex-recognition loop of the PAZ domain with guide RNA can be important in DNA silencing activity. Other prokaryotic Argonaute proteins (pAgos) can also be used in DNA interference and cleavage. The Argonaute proteins can be derived from Arabidopsis thaliana, D. melanogaster, Aquifex aeolicus, Thermus thermophiles, Pyrococcus furiosus, Thermus thermophilus JL-18, Thermus thermophilus strain HB27, Aquifex aeolicus strain VF5, Archaeoglobus fulgidus, Anoxybacillus flavithermus, Halogeometricum borinquense, Microsystis aeruginosa, Clostridium bartlettii, Halorubrum lacusprofundi, Thermosynechococcus elongatus, and Synechococcus elongatus. Argonaute proteins can also be used that are endo-nucleolytically inactive but post-translational modifications can be made to the conserved catalytic residues in order to activate them as endonucleases.

Human WRN is a RecQ helicase encoded by the Werner syndrome gene. It is implicated in genome maintenance, including replication, recombination, excision repair and DNA damage response. These genetic processes and expression of WRN are concomitantly upregulated in many types of cancers. Therefore, it has been proposed that targeted destruction of this helicase could be useful for elimination of cancer cells. Reports have applied the external guide sequence (EGS) approach in directing an RNase P RNA to efficiently cleave the WRN mRNA in cultured human cell lines, thus abolishing translation and activity of this distinctive 3′-5′ DNA helicase-nuclease.

The Class 2 type VI-A CRISPR/Cas effector “C2c2” demonstrates an RNA-guided RNase function. C2c2 from the bacterium Leptotrichia shahii provides interference against RNA phage. In vitro biochemical analysis show that C2c2 is guided by a single crRNA and can be programmed to cleave ssRNA targets carrying complementary protospacers. In bacteria, C2c2 can be programmed to knock down specific mRNAs. Cleavage is mediated by catalytic residues in the two conserved HEPN domains, mutations in which generate catalytically inactive RNA-binding proteins. The RNA-focused action of C2c2 complements the CRISPR-Cas9 system, which targets DNA, the genomic blueprint for cellular identity and function. The ability to target only RNA, which helps carry out the genomic instructions, offers the ability to specifically manipulate RNA in a high-throughput manner—and manipulate gene function more broadly. These results demonstrate the capability of C2c2 as a new RNA-targeting tools. C2c2 is preferably in a cloaked form.

Another Class 2 type V-B CRISPR/Cas effector “C2c1” can also be used in the present invention for editing DNA. C2c1 contains RuvC-like endonuclease domains related distantly to Cpf1 (described below). C2c1 can target and cleave both strands of target DNA site-specifically. According to Yang, et al. (PAM-Dependent Target DNA Recognition and Cleavage by C2c1 CRISPR-Cas Endonuclease, Cell, 2016 Dec. 15; 167(7):1814-1828)), a crystal structure confirms Alicyclobacillus acidoterrestris C2c1 (AacC2c1) binds to sgRNA as a binary complex and targets DNAs as ternary complexes, thereby capturing catalytically competent conformations of AacC2c1 with both target and non-target DNA strands independently positioned within a single RuvC catalytic pocket. Yang, et al. confirms that C2c1-mediated cleavage results in a staggered seven-nucleotide break of target DNA, crRNA adopts a pre-ordered five-nucleotide A-form seed sequence in the binary complex, with release of an inserted tryptophan, facilitating zippering up of 20-bp guide RNA:target DNA heteroduplex on ternary complex formation, and that the PAM-interacting cleft adopts a “locked” conformation on ternary complex formation. C2c1 is preferably in a cloaked form.

C2c3 is a gene editor effector of type V-C that is distantly related to C2c1, and also contains RuvC-like nuclease domains. C2c3 is also similar to the CasY.1-CasY.6 group described below. C2c3 is preferably in a cloaked form.

“CRISPR Cas9” as used herein refers to Clustered Regularly Interspaced Short Palindromic Repeat (CRISPR)-associated endonuclease Cas9. In bacteria the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately. Any of the Cas9 endonucleases are preferably in cloaked form.

CRISPR/Cpf1 is a DNA-editing technology analogous to the CRISPR/Cas9 system, characterized in 2015 by Feng Zhang's group from the Broad Institute and MIT. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. CRISPR/Cpf1 could have multiple applications, including treatment of genetic illnesses and degenerative conditions. As referenced above, Argonaute is another potential gene editing system. Cpf1 is preferably in cloaked form.

A CRISPR/TevCas9 system can also be used. In some cases it has been shown that once CRISPR/Cas9 cuts DNA in one spot, DNA repair systems in the cells of an organism will repair the site of the cut. The TevCas9 enzyme was developed to cut DNA at two sites of the target so that it is harder for the cells' DNA repair systems to repair the cuts (Wolfs, et al., Biasing genome-editing events toward precise length deletions with an RNA-guided TevCas9 dual nuclease, PNAS, doi:10.1073). The TevCas9 nuclease is a fusion of a I-Tevi nuclease domain to Cas9. TevCas9 is preferably in a cloaked form.

The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Alternatively, the wild type Streptococcus pyrogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GI:669193757; KM099232.1 GI:669193761; or KM099233.1 GI:669193765. Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765 or Cas9 amino acid sequence of PX330 or PX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine (2R,3S)-2-amino-3-methyl pentanoic acid and L-cyclopentyl glycine (S)-2-amino-2-cyclopentyl acetic acid. For other examples, one can consult textbooks or the worldwide web (a site is currently maintained by the California Institute of Technology and displays structures of non-natural amino acids that have been successfully incorporated into functional proteins). The Cas-9 can also be any shown in TABLE 1 below.

TABLE 1

| [symbol] riant No. | [symbol] r Alanine Substitution Mutants (compared to WT Cas9) | [symbol] sted* |
|---|---|---|
| | Cas9 N497A, R661A, Q695A, Q926A | S |
| | Cas9 N497A, R661A, Q695A, Q926A + D1135E | S |
| | Cas9 N497A, R661A, Q695A, Q926A + L169A | S |
| | Cas9 N497A, R661A, Q695A, Q926A + Y450A | S |
| | Cas9 N497A, R661A, Q695A, Q926A + M495A | [symbol] dicted |
| | Cas9 N497A, R661A, Q695A, Q926A + M694A | [symbol] dicted |
| | Cas9 N497A, R661A, Q695A, Q926A + H698A | [symbol] dicted |
| | Cas9 N497A, R661A, Q695A, Q926A + D1135E + L169A | [symbol] dicted |
| | Cas9 N497A, R661A, Q695A, O926A + D1135E + Y450A | [symbol] dicted |
| | Cas9 N497A, R661A, Q695A, Q926A + D1135E + M495A | [symbol] dicted |
| | Cas9 N497A, R661A, Q695A, Q926A + D1135E + M694A | [symbol] dicted |
| | Cas9 N497A, R661A, Q695A, Q926A + D1135E + M698A | [symbol] dicted |
| | [symbol] ee Alanine Substitution Mutants (compared to WT Cas9) | [symbol] sted* |
| | Cas9 R661A, Q695A, Q926A | (on target only) |
| | Cas9 R661A, Q695A, Q926A + D1135E | [symbol] dicted |
| | Cas9 R661A, Q695A, Q926A + L169A | [symbol] dicted |
| | Cas9 R661A, Q695A, Q926A + Y450A | [symbol] dicted |
| | Cas9 R661A, Q695A, Q926A + M495A | [symbol] dicted |
| | Cas9 R661A, Q695A, Q926A + M694A | [symbol] dicted |
| | Cas9 R661A, Q695A, Q926A + H698A | [symbol] dicted |
| | Cas9 R661A, Q695A, Q926A + D1135E + L169A | [symbol] dicted |
| | Cas9 R661A, Q695A, Q926A + D1135E + Y450A | [symbol] dicted |
| | Cas9 R661A, Q695A, Q926A + D1135E + M495A | [symbol] dicted |
| | Cas9 R661A, Q695A, Q926A + D1135E + M694A | [symbol] dicted |

[symbol]

indicates data missing or illegible when filed

Although the RNA-guided endonuclease Cas9 has emerged as a versatile genome-editing platform, some have reported that the size of the commonly used Cas9 from Streptococcus pyogenes (SpCas9) limits its utility for basic research and therapeutic applications that use the highly versatile adeno-associated virus (AAV) delivery vehicle. Accordingly, the six smaller Cas9 orthologues have been used and reports have shown that Cas9 from Staphylococcus aureus (SaCas9) can edit the genome with efficiencies similar to those of SpCas9, while being more than 1 kilobase shorter. SaCas9 is 1053 bp, whereas SpCas9 is 1358 bp.

The Cas9 nuclease sequence, or any of the gene editor effector sequences described herein, can be a mutated sequence. For example the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. In general, mutations of the gene editor effector sequence can minimize or prevent off-targeting.

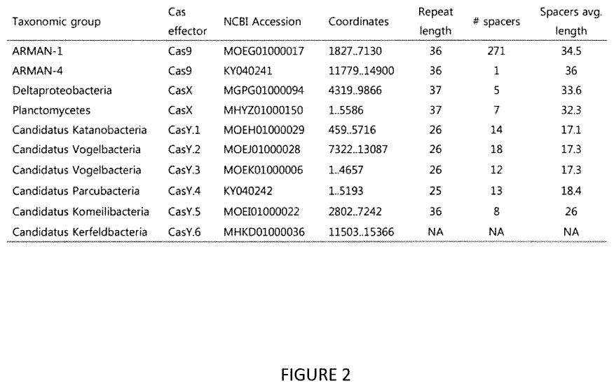

The gene editor effector can also be Archaea Cas9. The size of Archaea Cas9 is 950aa ARMAN 1 and 967aa ARMAN 4. The Archaea Cas9 can be derived from ARMAN-1 (Candidatus Micrarchaeum acidiphilum ARMAN-1) or ARMAN-4 (Candidatus Parvarchaeum acidiphilum ARMAN-4). Two examples of Archaea Cas9 are provided in FIG. 2, derived from ARMAN-1 and ARMAN-4. The sequences for ARMAN 1 and ARMAN 4 are below. Preferably, the Archaea Cas9 is cloaked.

ARMAN 1 amino acid sequence 950 aa
(SEQ ID NO: 1):
MRDSITAPRYSSALAARIKEENSAFKLGIDLGTKTGGVALVKDNKVLL
AKTFLDYHKQTLEERRIHRRNRRSRLARRKRIARLRSWILRQKIYGKQ
LPDPYKIKKMQLPNGVRKGENWIDLVVSGRDLSPEAFVRAITLIFQKR
GQRYEEVAKEIEEMSYKEFSTHIKALTSVTEEEFTALAAEIERRQDVV
DTDKEAERYTQLSELLSKVSESKSESKDRAQRKEDLGKVVNAFCSAHR
IEDKDKWCKELMKLLDRPVRHARFLNKVLIRCNICDRATPKKSRPDVR
ELLYFDTVRNFLKAGRVEQNPDVISYYKKIYMDAEVIRVKILNKEKLT
DEDKKQKRKLASELNRYKNKEYVTDAQKKMQEQLKTLLFMKLTGRSRY
CMAHLKERAAGKDVEEGLHGVVQKRHDRNIAQRNHDLRVINLIESLLF
DQNKSLSDAIRKNGLMYVTIEAPEPKTKHAKKGAAVVRDPRKLKEKLF
DDQNGVCIYTGLQLDKLEISKYEKDHIFPDSRDGPSIRDNLVLTTKEI
NSDKGDRTPWEWMHDNPEKWKAFERRVAEFYKKGRINERKRELLLNKG
TEYPGDNPTELARGGARVNNFITEFNDRLKTHGVQELQTIFERNKPIV
QVVRGEETQRLRRQWNALNQNFIPLKDRAMSFNHAEDAAIAASMPPKF
WREQIYRTAWHFGPSGNERPDFALAELAPQWNDFFMTKGGPIIAVLGK
TKYSWKHSIIDDTIYKPFSKSAYYVGIYKKPNAITSNAIKVLRPKLLN
GEHTMSKNAKYYHQKIGNERFLMKSQKGGSIITVKPHDGPEKVLQISP
TYECAVLIKHDGKIIVKFKPIKPLRDMYARGVIKAMDKELETSLSSMS
KHAKYKELHTHDIIYLPATKKHVDGYFIITKLSAKHGIKALPESMVKV
KYTQIGSENNSEVKLTKPKPEITLDSEDITNIYNFTR
ARMAN 1 nucleic acid sequence
(SEQ ID NO: 2):
atga gagactctat tactgcacct agatacagct ccgctcttgc
cgccagaata aaggagttta attctgcttt caagttagga
atcgacctag gaacaaaaac cggcggcgta gcactggtaa
aagacaacaa agtgctgctc gctaagacat tcctcgatta
ccataaacaa acactggagg aaaggaggat ccatagaaga
aacagaagga gcaggctagc caggcggaag aggattgctc
ggctgcgatc atggatactc agacagaaga tttatggcaa
gcagcttcct gacccataca aaatcaaaaa aatgcagttg
cctaatggtg tacgaaaagg ggaaaactgg attgacctgg
tagtttctgg acgggacctt tcaccagaag ccttcgtgcg
tgcaataact ctgatattcc aaaagagagg gcaaagatat
gaagaagtgg ccaaagagat agaagaaatg agttacaagg
aatttagtac tcacataaaa gccctgacat ccgttactga
agaagaattt actgctctgg cagcagagat agaacggagg
caggatgtgg ttgacacaga caaggaggcc gaacgctata
cccaattgtc tgagttgctc tccaaggtct cagaaagcaa
atctgaatct aaagacagag cgcagcgtaa ggaggatctc
ggaaaggtgg tgaacgcttt ctgcagtgct catcgtatcg
aagacaagga taaatggtgt aaagaactta tgaaattact
agacagacca gtcagacacg ctaggttcct taacaaagta
ctgatacgtt gcaatatctg cgatagggca acccctaaga
aatccagacc tgacgtgagg gaactgctat attttgacac
agtaagaaac ttcttgaagg ctggaagagt ggagcaaaac
ccagacgtta ttagttacta taaaaaaatt tatatggatg
cagaagtaat cagggtcaaa attctgaata aggaaaagct
gactgatgag gacaaaaagc aaaagaggaa attagcgagc
gaacttaaca ggtacaaaaa caaagaatac gtgactgatg
cgcagaagaa gatgcaagag caacttaaga cattgctgtt
catgaagctg acaggcaggt ctagatactg catggctcat
cttaaggaaa gggcagcagg caaagatgta gaagaaggac
ttcatggcgt tgtgcagaaa agacacgaca ggaacatagc
acagcgcaat cacgacttac gtgtgattaa tcttattgag
agtctgcttt tcgaccaaaa caaatcgctc tccgatgcaa
taaggaagaa cgggttaatg tatgttacta ttgaggctcc
agagccaaag actaagcacg caaagaaagg cgcagctgtg
gtaagggatc ccagaaagtt gaaggagaag ttgtttgatg
atcaaaacgg cgtttgcata tatacgggct tgcagttaga
caaattagag ataagtaaat acgagaagga ccatatcttt
ccagattcaa gggatggacc atctatcagg gacaatcttg
tactcactac aaaagagata aattcagaca aaggcgatag
gaccccatgg gaatggatgc atgataaccc agaaaaatgg
aaagcgttcg agagaagagt cgcagaattc tataagaaag
gcagaataaa tgagaggaaa agagaactcc tattaaacaa
aggcactgaa taccctggcg ataacccgac tgagctggcg
cggggaggcg cccgtgttaa caactttatt actgaattta
atgaccgcct caaaacgcat ggagtccagg aactgcagac
catctttgag cgtaacaaac caatagtgca ggtagtcagg
ggtgaagaaa cgcagcgtct gcgcagacaa tggaatgcac
taaaccagaa tttcatacca ctaaaggaca gggcaatgtc
gttcaaccac gctgaagacg cagccatagc agcaagcatg
ccaccaaaat tctggaggga gcagatatac cgtactgcgt
ggcactttgg acctagtgga aatgagagac cggactttgc
tttggcagaa ttggcgccac aatggaatga cttctttatg
actaagggcg gtccaataat agcagtgctg ggcaaaacga
agtatagttg gaagcacagc ataattgatg acactatata
caagccattc agcaaaagtg cttactatgt tgggatatac
aaaaagccga acgccatcac gtccaatgct ataaaagtct
taaggccaaa actcttaaat ggcgaacata caatgtctaa
gaatgcaaag tattatcatc agaagattgg taatgagcgc
ttcctcatga aatctcagaa aggtggatcg ataattacag
taaaaccaca cgacggaccg gaaaaagtgc ttcaaatcag
ccctacatat gaatgcgcag tccttactaa gcatgacggt
aaaataatag tcaaatttaa accaataaag ccgctacggg
acatgtatgc ccgcggtgtg attaaagcca tggacaaaga
gcttgaaaca agcctctcta gcatgagtaa acacgctaag
tacaaggagt tacacactca tgatatcata tatctgcctg
ctacaaagaa gcacgtagat ggctacttca taataaccaa
actaagtgcg aaacatggca taaaagcact ccccgaaagc
atggttaaag tcaagtatac tcaaattggg agtgaaaaca
atagtgaagt gaagcttacc aaaccaaaac cagagataac
tttggatagt gaagatatta caaacatata taatttcacc
cgctaag
ARMAN 4 amino acid sequence 967 aa
(SEQ ID NO: 3):
MLGSSRYLRYNLTSFEGKEPFLIMGYYKEYNKELSSKAQKEFNDQISE
ENSYYKLGIDLGDKTGIAIVKGNKIILAKTLIDLHSQKLDKRREARRN
RRTRLSRKKRLARLRSWVMRQKVGNQRLPDPYKIMHDNKYVVSIYNKS
NSANKKNWIDLLIHSNSLSADDFVRGLTIIFRKRGYLAFKYLSRLSDK
EFEKYIDNLKPPISKYEYDEDLEELSSRVENGEIEEKKFEGLKNKLDK
IDKESKDFQVKQREEVKKELEDLVDLFAKSVDNKIDKARWKRELNNLL
DKKVRKIRFDNRFILKCKIKGCNKNTPKKEKVRDFELKMVLNNARSDY
QISDEDLNSFRNEVINIFQKKENLKKGELKGVTIEDLRKQLNKTFNKA
KIKKGIREQIRSIVFEKISGRSKFCKEHLKEFSEKPAPSDRINYGVNS
AREQHDFRVLNFIDKKIFKDKLIDPSKLRYITIESPEPETEKLEKGQI
SEKSFETLKEKLAKETGGIDIVTGEKLKKDFEIEHIFPRARMGPSIRE
NEVASNLETNKEKADRTPVVEWFGQDEKRWSEFEKRVNSLYSKKKISE
RKREILLNKSNEYPGLNPTELSRIPSTLSDFVESIRKMFVKYGYEEPQ
TLVQKGKPIIQVVRGRDTQALRWRWHALDSNIIPEKDRKSSFNHAEDA
VIAACMPPYYLRQKIFREEAKIKRKVSNKEKEVTRPDMPTKKIAPNWS
EFMKTRNEPVIEVIGKVKPSWKNSIMDQTFYKYLLKPFKDNLIKIPNV
KNTYKWIGVNGQTDSLSLPSKVLSISNKKVDSSTVLLVHDKKGGKRNW
VPKSIGGLLVYITPKDGPKRIVQVKPATQGWYRNEDGRVDAVREFINP
VIEMYNNGKLAFVEKENEEELLKYFNLLEKGQKFERIRRYDMITYNSK
FYYVTKINKNHRVTIQEESKIKAESDKVKSSSGKEYTRKETEELSLQK
LAELISI
ARMAN 4 nucleic acid sequence
(SEQ ID NO: 4):
at gttaggctcc agcaggtacc tccgttataa cctaacctcg
tttgaaggca aggagccatt tttaataatg ggatattaca
aagagtataa taaggaatta agttccaaag ctcaaaaaga
atttaatgat caaatttctg aatttaattc gtattacaaa
ctaggtatag atctcggaga taaaacagga attgcaatcg
taaagggcaa caaaataatc ctagcaaaaa cactaattga
tttgcattcc caaaaattag ataaaagaag ggaagctaga
agaaatagaa gaactcggct ttccagaaag aaaaggcttg
cgagattaag atcgtgggta atgcgtcaga aagttggcaa
tcaaagactt cccgatccat ataaaataat gcatgacaat
aagtactggt ctatatataa taagagtaat tctgcaaata
aaaagaattg gatagatctg ttaatccaca gtaactcttt
atcagcagac gattttgtta gaggcttaac tataattttc
agaaaaagag gctatttagc atttaagtat ctttcaaggt
taagcgataa ggaatttgaa aaatacatag ataacttaaa
accacctata agcaaatacg agtatgatga ggatttagaa
gaattatcaa gcagggttga aaatggggaa atagaggaaa
agaaattcga aggcttaaag aataagctag ataaaataga
caaagaatct aaagactttc aagtaaagca aagagaagaa
gtaaaaaagg aactggaaga cttagttgat ttgtttgcta
aatcagttga taataaaata gataaagcta ggtggaaaag
ggagctaaat aatttattgg ataagaaagt aaggaaaata
cggtttgaca accgctttat tttgaagtgc aaaattaagg
gctgtaacaa gaatactcca aagaaagaga aggtcagaga
ttttgaattg aagatggttt taaataatgc tagaagcgat
tatcagattt ctgatgagga tttaaactct tttagaaatg
aagtaataaa tatatttcaa aagaaggaaa acttaaagaa
aggagagctg aaaggagtta ctattgaaga tttgagaaag
cagcttaata aaacttttaa taaagccaag attaaaaaag
ggataaggga gcagataagg tctatcgtgt ttgaaaaaat
tagtggaagg agtaaattct gcaaagaaca tctaaaagaa
ttttctgaga agccggctcc ttctgacagg attaattatg
gggttaattc agcaagagaa caacatgatt ttagagtctt
aaatttcata gataaaaaaa tattcaaaga taagttgata
gatccctcaa aattgaggta tataactatt gaatctccag
aaccagaaac agagaagttg gaaaaaggtc aaatatcaga
gaagagcttc gaaacattga aagaaaaatt ggctaaagaa
acaggtggta ttgatatata cactggtgaa aaattaaaga
aagactttga aatagagcac atattcccaa gagcaaggat
ggggccttct ataagggaaa acgaagtagc atcaaatctg
gaaacaaata aggaaaaggc cgatagaact ccttgggaat
ggtttgggca agatgaaaaa agatggtcag agtttgagaa
aagagttaat tctctttata gtaaaaagaa aatatcagag
agaaaaagag aaattttgtt aaataagagt aatgaatatc
cgggattaaa ccctacagaa ctaagtagaa tacctagtac
gctgagcgac ttcgttgaga gtataagaaa aatgtttgtt
aagtatggct atgaagagcc tcaaactttg gttcaaaaag
gaaaaccgat aatacaagtt gttagaggca gagacacaca
agctttgagg tggagatggc atgcattaga tagtaatata
ataccagaaa aggacaggaa aagttcattt aatcacgctg
aagatgcagt tattgccgcc tgtatgccac cttactatct
caggcaaaaa atatttagag aagaagcaaa aataaaaaga
aaagtaagca ataaggaaaa ggaagttaca cggcctgaca
tgcctactaa aaagatagct ccgaactggt cggaatttat
gaaaactaga aatgagccgg ttattgaagt aataggaaaa
gttaagccaa gctggaaaaa cagcataatg gatcaaacat
tttataaata tcttttgaag ccatttaaag ataacctgat
aaaaataccc aacgttaaaa atacatacaa gtggatagga
gttaatggac aaactgattc attatcectc ccgagtaagg
tcttatctat ctctaataaa aaggttgatt cttctacagt
tcttcttgtg catgataaga agggtggtaa gcggaattgg
gtacctaaaa gtataggggg tttgttggta tatataactc
ctaaagacgg gccgaaaaga atagttcaag taaagccagc
aactcagggt ttgttaatat atagaaatga agatggcaga
gtagatgctg taagagagtt cataaatcca gtgatagaaa
tgtataataa tggcaaattg gcatttgtag aaaaagaaaa
tgaagaagag cttttgaaat attttaattt gctggaaaaa
ggtcaaaaat ttgaaagaat aagacggtat gatatgataa
cctacaatag taaattttac tatgtaacaa aaataaacaa
gaatcacaga gttactatac aagaagagtc taagataaaa
gcagaatcag acaaagttaa gtcctcttca ggcaaagagt
atactcgtaa ggaaaccgag gaattatcac ttcaaaaatt
agcggaatta attagtatat aaaa

The gene editor effector can also be CasX, examples of which are shown in FIG. 2. CasX has a TTC PAM at the 5′ end (similar to Cpf1). The TTC PAM can have limitations in viral genomes that are GC rich, but not so much in those that are GC poor. The size of CasX (986 bp), smaller than other type V proteins, provides the potential for four gRNA plus one siRNA in a delivery plasmid. CasX can be derived from Deltaproteobacteria or Planctomycetes. The sequences for these CasX effectors are below. CasX is preferably in a cloaked form.

CasX.1 Planctomycetes amino acid sequence 978 aa
(SEQ ID NO: 5):
MQEIKRINKIRRRLVKDSNTKKAGKTGPMKTLLVRVMTPDLRERLENL
RKKPENIPQPISNTSRANLNKLLTDYTEMKKAILHVYWEEFQKDPVGL
MSRVAQPAPKNIDQRKLIPVKDGNERLTSSGFACSQCCQPLYVYKLEQ
VNDKGKPHTNYFGRCNVSEHERLILLSPHKPEANDELVTYSLGKFGQR
ALDFYSIHVTRESNHPVKPLEQIGGNSCASGPVGKALSDACMGAVASF
LTKYQDIILEHQKVIKKNEKRLANLKDIASANGLAFPKITLPPQPHTK
EGIEAYNNVVAQIVIWVNLNLWQKLKIGRDEAKPLQRLKGFPSFPLVE
RQANEVDWWDMVCNVKKLINEKKEDGKVFWQNLAGYKRQEALLPYLSS
EEDRKKGKKFARYQFGDLLLHLEKKHGEDWGKVYDEAWERIDKKVEGL
SKHIKLEEERRSEDAQSKAALTDWLRAKASFVIEGLKEADKDEFCRCE
LKLQKWYGDLRGKPFAIEAENSILDISGFSKQYNCAFIWQKDGVKKLN
LYLIINYFKGGKLRFKKIKPEAFEANRFYTVINKKSGEIVPMEVNENF
DDPNLIILPLAFGKRQGREFIWNDLLSLETGSLKLANGRVIEKTLYNR
RTRQDEPALFVALTFERREVLDSSNIKPMNLIGIDRGENIPAVIALTD
PEGCPLSRFKDSLGNPTHILRIGESYKEKORTIQAAKEVECIRRAGGY
SRKYASKAKNLADDMVRNTARDLLYYAVTQDAMLIFENLSRGFGRQGK
RTFMAERQYTRMEDWLTAKLAYEGLPSKTYLSKTLAQYTSKTCSNCGF
TITSADYDRVLEKLKKTATGWMTTINGKELKVEGQITYYNRYKRQNVV
KDLSVELDRLSEESVNNDISSWTKGRSGEALSLLKKRFSHRPVQEKFV
CLNCGFETHADEQAALNIARSWLFLRSQEYKKYQTNKTTGNTDKRAFV
ETWQSFYRKKLKEVWKPAV
CasX.1 Planctomycetes nucleic acid sequence
(SEQ ID NO: 6):
atgct tcttatttat cggagatatc ttcaaacacc
atcaacatgg caatggtgaa ccattaatat tctttgatgc
ttcttattta tcggagatat cttcaaacat tgcccatttt
acaggcatat cttctggctc tttgatgctt cttatttatc
ggagatatct tcaaacgtaa tgtattgaga aagacatcaa
gattagataa ctttgatgct tcttatttat cggagatatc
ttcaaacaca gaaacctgca aagattgtat atatataagc
tttgatgctt cttatttatc ggagatatct tcaaacgata
cgtattttag cccgtctatt tggggattaa ctttgatgct
tcttatttat cggagatatc ttcaaacccc gcatatccag
atttttcaat gacttctgga aattgtattt tcaatatttt
acaagttgcg gaggatacct ttaataattt agcagagtta
cgcactgtaa acctgttctt ctcacaaaaa gctttaacat
cagattttca aagaacttct tatgtaattt ataagaatct
aaaaaaacag ctctgggttt gcatccagaa ctctccgata
aataagcgct ttacccatac gacatagtcg ctggtgatgg
ctctcaaagt aatgagataa aagcgccagt aataatttac
tattcacaaa tcctttcgtc aagcttaaaa tcaatcaaag
accatatccc cttcattcca aatagcagcg cttccgtacc
tttctatccg ttcatatatc tcctctgaga gaggataaat
taccagactt atagagccat ccataaatcc tttttcttta
aggttgagct ttagatcagc ccaccttgct tttgaaaggt
taaactcaaa gacagaatat tgaatccgaa
caccataggc ttccagaagt ttaactaacc gtgccctgac
cttatcatct tcaatatcat aacaaatgag atgtcgcatt
ttaaagctct ataggcttat aacattccct atcatcttga
atatgctggc taaacaacct aacctgccgc tcaactgcgt
gctgatacgt tattgattgg ataagtaaat tggttttctg
ctcatctacc ttaaagaatt gatgccattt tttgattact
tttggatagg catccttatt cagccaaaca cctttttggt
cagtttcttt cctgaaatcg tctgtatcca cttcccttct
atttatcaaa ttgatcacaa aacggtcagc caacggccgc
cactcctcca gaagatcgca tattaaagag ggacgaccat
aatagacgtc atgcaagtaa ccaaaggccg ggtcaaaacc
gacgagtaat gcagtcgaat gtatttcgtt gaacaggagg
gtgtagataa ggctcatcat ggcgttgatt tcatcctcag
gaggtctctt ggtacggcgc acaaaaacaa agcttggatg
ctttaagata gccgaaaaat tgccataata ctgccttgtt
gttgcgcctt ctattccacg caaggtctct aaatcagtga
cggcgttgat ttcggtacac tcgattctca aaccaagtct
atatttatca agtaatgatt gctggttttt gatcttaccg
gcaacgatac tttttgcaat ttcaagtttt ttgtggggat
caaaatgctt atgaatttgc gcccgacgaa taaacagatt
tttgacgggt tcaaattgaa ggctcccttg atattcccat
ctgccgctaa agaaatgtat cggtatagat tattctctgc
aaaggctaat aacacggcta tcgagggtaa cccggccaac
taccacgata tcttttacct tcattgcggg aatcttctgc
cccttctctt cattgtcctt ttttatgaga aatgcccgac
cacgacaatc caaaatgaat tcatcacccg tgagatagag
ggttatcctg tcggttatag cggtcatcag taagcctttt
atttttctaa ccaagtattg aaggaagaca cgattcacta
tactggcact gcggacacct atggtcatca accttgggaa
acctgcttat atcaaaggac aagaagcagt ctcgcagatt
tgtaacaact tctacacaac gcactttcag ggttttatct
ataacaattt ctttccgtct ccgtgtttca cagaaaaata
tttcaccaac tggtatattg acattataca tctcttcaag
gcaaattgcc tgtaacccaa tctgaacgtg gaagttctca
aaatccctta ccttccctgt ctttgtttcg ataggaatcg
gtatcccatc cctccactcg ataaggtctg cccggcctgc
caaaccgagc ttattgctgt aaagatacac gcctgttacc
tgcttacaat cagggcagct tctctgcgat gatttatcca
ccgccctgtg cgcgtgtatg gcctctgtaa agtggatgct
cttagccata ttacgccgtt ctccaacaaa ggcataccat
gcattgcgcg gacaatagat tgactccatt accgtgctga
tgtgcaatat cagacggctg gtttccatac ttctttgagc
ttctttctgt aaaaggattg ccatgtttca acaaatgccc
ttttgtcagt atttccggtc gttttattgg
tttgatacttcttatattct tgagaacgga gaaagagcca
cgaccttgca atattcagtg ctgcttgttc gtctgcatgg
gtttcaaaac cacagttcag gcaaacaaac ttttcctgca
ccggcctgtg actaaatctc ttttttagca gagataaagc
ttcaccactg cggccttttg tccaactaga aatatcatta
tttaccgact cttccgaaag tctatccagc tctacagaga
ggtcttttac cacattctgc cttttatacc ggttatagta
tgttatctgt ccttcaactt ttaactcttt tccattgatt
gtagtcatcc atccagtagc cgtcttcttg agcttttcga
gcaccctgtc ataatctgca cttgtgattg taaaaccaca
attagaacat gtctttgagg tatactgtgc cagagtcttt
gaaagatagg tttttgatgg cagaccttca taggcaagct
ttgcagtcag ccagtcttcc atcctcgtgt actgcctttc
cgccataaaa gtcctcttgc cttgtctacc aaaaccgcgg
gaaagatttt caaaaatgag cattgcatct tgagtaacag
cataatataa gaggtcacga gctgtatttc ttaccatatc
gtccgccaga ttcttcgcct ttgatgcata ttttctcgaa
tatccgcctg cccgcctttg ttcaacttct ttagcagcct
gaatagtccg ttgtttttcc ttataacttt ctcctattcg
caaaatatgc gttggattgc ccaatgaatc tttgaatctt
gacaaggggc atccttccgg gtctgttaat gctatgactg
ccgggatatt ttctccccgg tctattccta tcagattcat
cggttttata ttcgatgagt caagcacctc tcttctttca
aatgtcaggg caacaaaaag tgctggttca tcctgtctcg
tccttctgtt atagagcgtt ttttcaataa ccctgccatt
ggcgagtttc aatgaacccg tctcaaggct caataggtcg
ttccagataa actccctccc ctgccttttt ccaaaggcca
aaggcagaat tatcaaattc gggtcatcaa aattgaagtt
gacctccata ggcacaatct caccgctttt tttattaatt
actgtataaa acctatttgc ttcaaaagct tctggcttga
tttttttgaa gcgtagctta ccacctttga agtaatttat
tattaaataa agatttaact tctttacgcc gtctttctgc
catataaatg cacaattata ctgtttagaa aatccgctta
tatctaaaat gctgttctct gcttctatag caaatggttt
tcctctcaaa tctccatacc acttttgaag ctttaactca
cacctgcaaa actcatcctt atcagcttct ttgagccctt
caataacaaa agaggccttt gccctgagcc aatcagtgag
ggcagccttt gattgagcat cttcagacct tctttcttcc
tccaacttta tgtgcttact cagaccttca acttttttat
ctattctttc ccatgcctca tcataaactt tgccccaatc
ttcaccgtgt ttcttttcaa ggtgaagcaa aaggtcacca
aactgataac gcgcaaactt ttttcctttt ttacggtctt
cttcagacga aagatatgga agcaaggctt cctgcctttt
atatccagca agattttgcc agaagacctt cccgtcctct
ttcttttcgt taatcaactt tttgacatta cagaccatat
cccaccaatc aacctcattc gcctggcgtt caacaagagg
gaaggacgga aaacccttaa gccgctgtaa gggctttgcc
tcatccctgc caattttgag tttctgccaa agattcaggt
ttacccagat cactatctga gcaacaacat tgttataagc
ttcaatccct tcttttgtat gcggttgcgg tggaagagtg
attttaggaa atgcaagccc gtttgcactt gctatatcct
ttagatttgc caatctcttt tcgttttttt ttataacctt
ttggtgttcg aggatgatgt cctggtactt tgtaaggaaa
ctggctactg ctcccataca ggcatcagat aaagccttac
caacgggacc acttgcgcag ctattgccac cgatctgttc
tagcggcttt acaggatggt tcgattctct tgttacgtgg
attgaataaa agtccaatgc cctttgaccg aacttcccca
acgaatacgt tactagctcg tcatttgcct ccggtttatg
cggcgagagc aatatcaaac gttcatgctc ggagacatta
caacggccaa agtaatttgt atggggctta cccttgtcat
tcacttgttc aagcttataa acatagaggg gttgacagca
ctgagaacag gcaaatccag aacttgttag tctctcattt
ccgtccttca ccggaatcaa ttttctctga tcaatattct
tgggcgctgg ttgtgcaacc ctgctcatca atccgacagg
gtctttttgg aactcttccc aataaacatg caggattgct
ttcttcattt ccgtatagtc agtgaggagt ttatttaaat
ttgcacgtga agtatttgaa atgggctgag gaatgttttc
cggctttttg cgaagattct ctaacctttc tctcaggtca
ggtgtcataa cccgaacgag caaggttttc atagggccgg
ttttgccggc ttttttcgtg ttgctatcct ttaccaatct
ccttcgtatt ttatttatcc tttttatttc ctgcatcttt
CasX.1 Deltaproteobacteria amino acid sequence
986 aa
(SEQ ID NO: 7):
MEKRINKIRKKLSADNATKPVSRSGPMKTLLVRVMTDDLKKRLEKRRK
KPEVMPQVISNNAANNLRMLLDDYTKMKEAILQVYWQEFKDDHVGLMC
KFAQPASKKIDONKLKPEMDEKGNLTTAGFACSQCGQPLFVYKLEQVS
EKGKAYTNYFGRCNVAEHEKLILLAQLKPEKDSDEAVTYSLGKFGQRA
LDFYSIHVTKESTHPVKPLAQIAGNRYASGPVGKALSDACMGTIASFL
SKYQDIIIEHQKVVKGNQKRLESLRELAGKENLEYPSVTLPPQPHTKE
GVDAYNEVIARVRMWVNLNLWQKLKLSRDDAKPLLRLKGFPSFPVVER
RENEVDWWNTINEVKKLIDAKRDMGRVFWSGVTAEKRNTILEGYNYLP
NENDHKKREGSLENPKKPAKRQFGDLLLYLEKKYAGDWGKVFDEAWER
IDKKIAGLTSHIEREEARNAEDAQSKAVLTDWLRAKASFVLERLKEMD
EKEFYACEIQLQKWYGDLRGNPFAVEAENRVVDISGFSIGSDGHSIQY
RNLLAWKYLENGKREFYLLMNYGKKGRIRFTDGTDIKKSGKWQGLLYG
GGKAKVIDLTFDPDDEQLIILPLAFGTRQGREFIWNDLLSLETGLIKL
ANGRVIEKTIYNKKIGRDEPALFVALTFERREVVDPSNIKPVNLIGVD
RGENIPAVIALTDPEGCPLPEFKDSSGGPTDILRIGEGYKEKQRAIQA
AKEVEQRRAGGYSRKFASKSRNLADDMVRNSARDLFYHAVTHDAVLVF
ENLSRGFGRQGKRTFMTERQYTKMEDWLTAKLAYEGLTSKTYLSKTLA
QYTSKTCSNCGFTITTADYDGMLVRLKKTSDGWATTLNNKELKAEGQI
TYYNRYKRQTVEKELSAELDRLSEESGNNDISKVVTKGRRDEALFLLK
KRFSHRPVQEQFVCLDCGHEVHADEQAALNIARSWLFLNSNSTEFKSY
KSGKQPFVGAWQAFYKRRLKEVWKPNA
CasX.1 Deltaproteobacteria nucleic acid sequence
(SEQ ID NO: 8):
at ggaaaagaga ataaacaaga
tacgaaagaa actatcggcc gataatgcca caaagcctgt
gagcaggagc ggccccatga aaacactcct tgtccgggtc
atgacggacg acttgaaaaa aagactggag aagcgtcgga
aaaagccgga agttatgccg caggttattt caaataacgc
agcaaacaat cttagaatgc tccttgatga ctatacaaag
atgaaggagg cgatactaca agtttactgg caggaattta
aggacgacca tgtgggcttg atgtgcaaat ttgcccagcc
tgcttccaaa aaaattgacc agaacaaact aaaaccggaa
atggatgaaa aaggaaatct aacaactgcc ggttttgcat
gttctcaatg cggtcagccg ctatttgttt ataagcttga
acaggtgagt gaaaaaggca aggcttatac aaattacttc
ggccggtgta atgtggccga gcatgagaaa ttgattcttc
ttgctcaatt aaaacctgaa aaagacagtg acgaagcagt
gacatactcc cttggcaaat tcggccagag ggcattggac
ttttattcaa tccacgtaac aaaagaatcc acccatccag
taaagcccct ggcacagatt gcgggcaacc gctatgcaag
cggacctgtt ggcaaggccc tttccgatgc ctgtatgggc
actatagcca gttttctttc gaaatatcaa gacatcatca
tagaacatca aaaggttgtg aagggtaatc aaaagaggtt
agagagtctc agggaattgg cagggaaaga aaatcttgag
tacccatcgg ttacactgcc gccgcagccg catacgaaag
aaggggttga cgcttataac gaagttattg caagggtacg
tatgtgggtt aatcttaatc tgtggcaaaa gctgaagctc
agccgtgatg acgcaaaacc gctactgcgg ctaaaaggat
tcccatcttt ccctgttgtg gagcggcgtg aaaacgaagt
tgactggtgg aatacgatta atgaagtaaa aaaactgatt
gacgctaaac gagatatggg acgggtattc tggagcggcg
ttaccgcaga aaagagaaat accatccttg aaggatacaa
ctatctgcca aatgagaatg accataaaaa gagagagggc
agtttggaaa accctaagaa gcctgccaaa cgccagtttg
gagacctctt gctgtatctt gaaaagaaat atgccggaga
ctggggaaag gtcttcgatg aggcatggga gaggatagat
aagaaaatag ccggactcac aagccatata gagcgcgaag
aagcaagaaa cgcggaagac gctcaatcca aagccgtact
tacagactgg ctaagggcaa aggcatcatt tgttcttgaa
agactgaagg aaatggatga aaaggaattc tatgcgtgtg
aaatccaact tcaaaaatgg tatggcgatc ttcgaggcaa
cccgtttgcc gttgaagctg agaatagagt tgttgatata
agcgggtttt ctatcggaag cgatggccat tcaatccaat
acagaaatct ccttgcctgg aaatatctgg agaacggcaa
gcgtgaattc tatctgttaa tgaattatgg caagaaaggg
cgcatcagat ttacagatgg aacagatatt aaaaagagcg
gcaaatggca gggactatta tatggcggtg gcaaggcaaa
ggttattgat ctgactttcg accccgatga tgaacagttg
ataatcctgc cgctggcctt tggcacaagg caaggccgcg
agtttatctg gaacgatttg ctgagtcttg aaacaggcct
gataaagctc gcaaacggaa gagttatcga aaaaacaatc
tataacaaaa aaatagggcg ggatgaaccg gctctattcg
ttgccttaac atttgagcgc cgggaagttg ttgatccatc
aaatataaag cctgtaaacc ttataggcgt tgaccgcggc
gaaaacatcc cggcggttat tgcattgaca gaccctgaag
gttgtccttt accggaattc aaggattcat cagggggccc
aacagacatc ctgcgaatag gagaaggata taaggaaaag
cagagggcta ttcaggcagc aaaggaggta gagcaaaggc
gggctggcgg ttattcacgg aagtttgcat ccaagtcgag
gaacctggcg gacgacatgg tgagaaattc agcgcgagac
cttttttacc atgccgttac ccacgatgcc gtccttgtct
ttgaaaacct gagcaggggt tttggaaggc agggcaaaag
gaccttcatg acggaaagac aatatacaaa gatggaagac
tggctgacag cgaagctcgc atacgaaggt cttacgtcaa
aaacctacct ttcaaagacg ctggcgcaat atacgtcaaa
aacatgctcc aactgcgggt ttactataac gactgccgat
tatgacggga tgttggtaag gcttaaaaag acttctgatg
gatgggcaac taccctcaac aacaaagaat taaaagccga
aggccagata acgtattata accggtataa aaggcaaacc
gtggaaaaag aactctccgc agagcttgac aggctttcag
aagagtcggg caataatgat atttctaagt ggaccaaggg
tcgccgggac gaggcattat ttttgttaaa gaaaagattc
agccatcggc ctgttcagga acagtttgtt tgcctcgatt
gcggccatga agtccacgcc gatgaacagg cagccttgaa
tattgcaagg tcatggcttt ttctaaactc aaattcaaca
gaattcaaaa gttataaatc gggtaaacag cccttcgttg
gtgcttggca ggccttttac aaaaggaggc ttaaagaggt
atggaagccc aacgcctgat

The gene editor effector can also be CasY.1-CasY.6, examples of which are shown in FIG. 2. CasY.1-CasY.6 has TA PAM, and a shorter PAM sequence can be useful as there are less targeting limitations. The size of CasY.1-CasY.6 (1125 bp) provides the potential for two gRNA plus one siRNA or four gRNA in a delivery plasmid. CasY.1-CasY.6 can be derived from phyla radiation (CPR) bacteria, such as, but not limited to, katanobacteria, vogelbacteria, parcubacteria, komeilibacteria, or kerfeldbacteria The sequences for CasY.1-CasY.6 are below. CasY.1-CasY.6 are preferably in a cloaked form.

CasY.1 Candidatus katanobacteria amino acid sequence 1125 aa
(SEQ ID NO: 9):
MRKKLFKGYILHNKRLVYTGKAAIRSIKYPLVAPNKTALNNLSEKIIYDYEHLFGP
LNVASYARNSNRYSLVDFWIDSLRAGVIWQSKSTSLIDLISKLEGSKSPSEKIFEQIDFELKNKL
DKEQFKDIILLNTGIRSSSNVRSLRGRFLKCFKEEFRDTEEVIACVDKWSKDLIVEGKSILVSKQ
FLYWEEEFGIKIFPHFKDNHDLPKLTFFVEPSLEFSPHLPLANCLERLKKFDISRESLLGLDNNF
SAFSNYFNELFNLLSRGEIKKIVTAVLAVSKSWENEPELEKRLHFLSEKAKLLGYPKLTSSWAD
YRMIIGGKIKSWHSNYTEQLIKVREDLKKHQIALDKLQEDLKKVVDSSLREQIEAQREALLPLLD
TMLKEKDFSDDLELYRFILSDFKSLLNGSYQRYIQTEEERKEDRDVTKKYKDLYSNLRNIPRFF
GESKKEQFNKFINKSLPTIDVGLKILEDIRNALETVSVRKPPSITEEYVTKQLEKLSRKYKINAFN
SNRFKQITEQVLRKYNNGELPKISEVFYRYPRESHVAIRILPVKISNPRKDISYLLDKYQISPDW
KNSNPGEVVDLIEIYKLTLGWLLSCNKDFSMDFSSYDLKLFPEAASLIKNFGSCLSGYYLSKMIF
NCITSEIKGMITLYTRDKFVVRYVTQMIGSNQKFPLLCLVGEKQTKNFSRNWGVLIEEKGDLGE
EKNQEKCLIFKDKTDFAKAKEVEIFKNNIWRIRTSKYQIQFLNRLFKKTKEWDLMNLVLSEPSLV
LEEEWGVSWDKDKLLPLLKKEKSCEERLYYSLPLNLVPATDYKEQSAEIEQRNTYLGLDVGEF
GVAYAVVRIVRDRIELLSWGFLKDPALRKIRERVQDMKKKQVMAVFSSSSTAVARVREMAIHS
LRNQIHSIALAYKAKIIYEISISNFETGGNRMAKIYRSIKVSDVYRESGADTLVSEMIWGKKNKQ
MGNHISSYATSYTCCNCARTPFELVIDNDKEYEKGGDEFIFNVGDEKKVRGFLQKSLLGKTIK
GKEVLKSIKEYARPPIREVLLEGEDVEQLLKRRGNSYIYRCPFCGYKTDADIQAALNIACRGYIS
DNAKDAVKEGERKLDYILEVRKLWEKNGAVLRSAKFL
CasY.1 Candidatus katanobacteria nucleic acid sequence
(SEQ ID NO: 10):
at gcgcaaaaaa ttgtttaagg gttacatttt acataataag aggcttgtat atacaggtaa agctgcaata
cgttctatta aatatccatt agtcgctcca aataaaacag ccttaaacaa tttatcagaa aagataattt atgattatga
gcatttattc ggacctttaa atgtggctag ctatgcaaga aattcaaaca ggtacagcct tgtggatttt tggatagata
gcttgcgagc aggtgtaatt tggcaaagca aaagtacttc gctaattgat ttgataagta agctagaagg atctaaatcc
ccatcagaaa agatatttga acaaatagat tttgagctaa aaaataagtt ggataaagag caattcaaag atattattct
tcttaataca ggaattcgtt ctagcagtaa tgttcgcagt ttgagggggc gctttctaaa gtgttttaaa gaggaattta
gagataccga agaggttatc gcctgtgtag ataaatggag caaggacctt atcgtagagg gtaaaagtat actagtgagt
aaacagtttc tttattggga agaagagttt ggtattaaaa tttttcctca ttttaaagat aatcacgatt taccaaaact aacttttttt
gtggagcctt ccttggaatt tagtccgcac ctccctttag ccaactgtct tgagcgtttg aaaaaattcg atatttcgcg
tgaaagtttg ctcgggttag acaataattt ttcggccttt tctaattatt tcaatgagct ttttaactta ttgtccaggg gggagattaa
aaagattgta acagctgtcc ttgctgtttc taaatcgtgg gagaatgagc cagaattgga aaagcgctta cattttttga
gtgagaaggc aaagttatta gggtacccta agcttacttc ttcgtgggcg gattatagaa tgattattgg cggaaaaatt
aaatcttggc attctaacta taccgaacaa ttaataaaag ttagagagga cttaaagaaa catcaaatcg cccttgataa
attacaggaa gatttaaaaa aagtagtaga tagctcttta agagaacaaa tagaagctca acgagaagct ttgcttcctt
tgcttgatac catgttaaaa gaaaaagatt tttccgatga tttagagctt tacagattta tcttgtcaga ttttaagagt ttgttaaatg
ggtcttatca aagatatatt caaacagaag aggagagaaa ggaggacaga gatgttacca aaaaatataa agatttatat
agtaatttgc gcaacatacc tagatttttt ggggaaagta aaaaggaaca attcaataaa tttataaata aatctctccc
gaccatagat gttggtttaa aaatacttga ggatattcgt aatgctctag aaactgtaag tgttcgcaaa cccccttcaa
taacagaaga gtatgtaaca aagcaacttg agaagttaag tagaaagtac aaaattaacg cctttaattc aaacagattt
aaacaaataa ctgaacaggt gctcagaaaa tataataacg gagaactacc aaagatctcg gaggtttttt atagataccc
gagagaatct catgtggcta taagaatatt acctgttaaa ataagcaatc caagaaagga tatatcttat cttctcgaca
aatatcaaat tagccccgac tggaaaaaca gtaacccagg agaagttgta gatttgatag agatatataa attgacattg
ggttggctct tgagttgtaa caaggatttt tcgatggatt tttcatcgta tgacttgaaa ctcttcccag aagccgcttc
cctcataaaa aattttggct cttgcttgag tggttactat ttaagcaaaa tgatatttaa ttgcataacc agtgaaataa
aggggatgat tactttatat actagagaca agtttgttgt tagatatgtt acacaaatga taggtagcaa tcagaaattt
cctttgttat gtttggtggg agagaaacag actaaaaact tttctcgcaa ctggggtgta ttgatagaag agaagggaga
tttgggggag gaaaaaaacc aggaaaaatg tttgatattt aaggataaaa cagattttgc taaagctaaa gaagtagaaa
tttttaaaaa taatatttgg cgtatcagaa cctctaagta ccaaatccaa tttttgaata ggctttttaa gaaaaccaaa
gaatgggatt taatgaatct tgtattgagc gagcctagct tagtattgga ggaggaatgg ggtgtttcgt gggataaaga
taaactttta cctttactga agaaagaaaa atcttgcgaa gaaagattat attactcact tccccttaac ttggtgcctg
ccacagatta taaggagcaa tctgcagaaa tagagcaaag gaatacatat ttgggtttgg atgttggaga atttggtgtt
gcctatgcag tggtaagaat agtaagggac agaatagagc ttctgtcctg gggattcctt aaggacccag ctcttcgaaa
aataagagag cgtgtacagg atatgaagaa aaagcaggta atggcagtat tttctagctc ttccacagct gtcgcgcgag
tacgagaaat ggctatacac tctttaagaa atcaaattca tagcattgct ttggcgtata aagcaaagat aatttatgag
atatctataa gcaattttga gacaggtggt aatagaatgg ctaaaatata ccgatctata aaggtttcag atgtttatag
ggagagtggt gcggataccc tagtttcaga gatgatctgg ggcaaaaaga ataagcaaat gggaaaccat atatcttcct
atgcgacaag ttacacttgt tgcaattgtg caagaacccc ttttgaactt gttatagata atgacaagga atatgaaaag
ggaggcgacg aatttatttt taatgttggc gatgaaaaga aggtaagggg gtttttacaa aagagtctgt taggaaaaac
aattaaaggg aaggaagtgt tgaagtctat aaaagagtac gcaaggccgc ctataaggga agtcttgctt gaaggagaag
atgtagagca gttgttgaag aggagaggaa atagctatat ttatagatgc cctttttgtg gatataaaac tgatgcggat
attcaagcgg cgttgaatat agcttgtagg ggatatattt cggataacgc aaaggatgct gtgaaggaag gagaaagaaa
attagattac attttggaag ttagaaaatt gtgggagaag aatggagctg ttttgagaag cgccaaattt ttatagtt
CasY.2 Candidatus vogelbacteria amino acid sequence 1226 aa
(SEQ ID NO: 11):
MQKVRKTLSEVHKNPYGTKVRNAKTGYSLQIERLSYTGKEGMRSFKIPLENKN
KEVFDEFVKKIRNDYISQVGLLNLSDWYEHYQEKQEHYSLADFWLDSLRAGVIFAHKETEIKNL
ISKIRGDKSIVDKFNASIKKKHADLYALVDIKALYDFLTSDARRGLKTEEEFFNSKRNTLFPKFRK
KDNKAVDLWVKKFIGLDNKDKLNFTKKFIGFDPNPQIKYDHTFFFHQDINFDLERITTPKELIST
YKKFLGKNKDLYGSDETTEDQLKMVLGFHNNHGAFSKYFNASLEAFRGRDNSLVEQIINNSPY
WNSHRKELEKRIIFLQVQSKKIKETELGKPHEYLASFGGKFESWVSNYLRQEEEVKRQLFGYE
ENKKGQKKFIVGNKQELDKIIRGTDEYEIKAISKETIGLTQKCLKLLEQLKDSVDDYTLSLYRQLI
VELRIRLNVEFQETYPELIGKSEKDKEKDAKNKRADKRYPQIFKDIKLIPNFLGETKQMVYKKFI
RSADILYEGINFIDQIDKQITQNLLPCFKNDKERIEFTEKQFETLRRKYYLMNSSRFHHVIEGIIN
NRKLIEMKKRENSELKTFSDSKFVLSKLFLKKGKKYENEVYYTFYINPKARDQRRIKIVLDINGN
NSVGILQDLVQKLKPKWDDIIKKNDMGELIDAIEIEKVRLGILIALYCEHKFKIKKELLSLDLFASA
YQYLELEDDPEELSGTNLGRFLQSLVCSEIKGAINKISRTEYIERYTVQPMNTEKNYPLLINKEG
KATWHIAAKDDLSKKKGGGTVAMNQKIGKNFFGKQDYKTVFMLQDKRFDLLTSKYHLQFLSK
TLDTGGGSWWKNKNIDLNLSSYSFIFEQKVKVEWDLTNLDHPIKIKPSENSDDRRLFVSIPFVI
KPKQTKRKDLQTRVNYMGIDIGEYGLAWTIINIDLKNKKINKISKQGFIYEPLTHKVRDYVATIKD
NQVRGTFGMPDTKLARLRENAITSLRNQVHDIAMRYDAKPVYEFEISNFETGSNKVKVIYDSV
KRADIGRGQNNTEADNTEVNLVWGKTSKQFGSQIGAYATSYICSFCGYSPYYEFENSKSGDE
EGARDNLYQMKKLSRPSLEDFLQGNPVYKTFRDFDKYKNDQRLQKTGDKDGEWKTHRGNT
AIYACQKCRHISDADIQASYWIALKQVVRDFYKDKEMDGDLIQGDNKDKRKVNELNRLIGVHK
DVPIINKNLITSLDINLL
CasY.2 Candidatus vogelbacteria nucleic acid sequence
(SEQ ID NO: 12):
a tggtattagg ttttcataat aatcacggcg ctttttctaa gtatttcaac gcgagcttgg aagcttttag
ggggagagac aactccttgg ttgaacaaat aattaataat tctccttact ggaatagcca tcggaaagaa ttggaaaaga
gaatcatttt tttgcaagtt cagtctaaaa aaataaaaga gaccgaactg ggaaagcctc acgagtatct tgcgagtttt
ggcgggaagt ttgaatcttg ggtttcaaac tatttacgtc aggaagaaga ggtcaaacgt caactttttg gttatgagga
gaataaaaaa ggccagaaaa aatttatcgt gggcaacaaa caagagctag ataaaatcat cagagggaca gatgagtatg
agattaaagc gatttctaag gaaaccattg gacttactca gaaatgttta aaattacttg aacaactaaa agatagtgtc
gatgattata cacttagcct atatcggcaa ctcatagtcg aattgagaat cagactgaat gttgaattcc aagaaactta
tccggaatta atcggtaaga gtgagaaaga taaagaaaaa gatgcgaaaa ataaacgggc agacaagcgt
tacccgcaaa tttttaagga tataaaatta atccccaatt ttctcggtga aacgaaacaa atggtatata agaaatttat
tcgttccgct gacatccttt atgaaggaat aaattttatc gaccagatcg ataaacagat tactcaaaat ttgttgcctt
gttttaagaa cgacaaggaa cggattgaat ttaccgaaaa acaatttgaa actttacggc gaaaatacta tctgatgaat
agttcccgtt ttcaccatgt tattgaagga ataatcaata ataggaaact tattgaaatg aaaaagagag aaaatagcga
gttgaaaact ttctccgata gtaagtttgt tttatctaag ctttttctta aaaaaggcaa aaaatatgaa aatgaggtct attatacttt
ttatataaat ccgaaagctc gtgaccagcg acggataaaa attgttcttg atataaatgg gaacaattca gtcggaattt
tacaagatct tgtccaaaag ttgaaaccaa aatgggacga catcataaag aaaaatgata tgggagaatt aatcgatgca
atcgagattg agaaagtccg gctcggcatc ttgatagcgt tatactgtga gcataaattc aaaattaaaa aagaactctt
gtcattagat ttgtttgcca gtgcctatca atatctagaa ttggaagatg accctgaaga actttctggg acaaacctag
gtcggttttt acaatccttg gtctgctccg aaattaaagg tgcgattaat aaaataagca ggacagaata tatagagcgg
tatactgtcc agccgatgaa tacggagaaa aactatcctt tactcatcaa taaggaggga aaagccactt ggcatattgc
tgctaaggat gacttgtcca agaagaaggg tgggggcact gtcgctatga atcaaaaaat cggcaagaat ttttttggga
aacaagatta taaaactgtg tttatgcttc aggataagcg gtttgatcta ctaacctcaa agtatcactt gcagttttta
tctaaaactc ttgatactgg tggagggtct tggtggaaaa acaaaaatat tgatttaaat ttaagctctt attctttcat tttcgaacaa
aaagtaaaag tcgaatggga tttaaccaat cttgaccatc ctataaagat taagcctagc gagaacagtg atgatagaag
gcttttcgta tccattcctt ttgttattaa accgaaacag acaaaaagaa aggatttgca aactcgagtc aattatatgg
ggattgatat cggagaatat ggtttggctt ggacaattat taatattgat ttaaagaata aaaaaataaa taagatttca
aaacaaggtt tcatctatga gccgttgaca cataaagtgc gcgattatgt tgctaccatt aaagataatc aggttagagg
aacttttggc atgcctgata cgaaactagc cagattgcga gaaaatgcca ttaccagctt gcgcaatcaa gtgcatgata
ttgctatgcg ctatgacgcc aaaccggtat atgaatttga aatttccaat tttgaaacgg ggtctaataa agtgaaagta
atttatgatt cggttaagcg agctgatatc ggccgaggcc agaataatac cgaagcagac aatactgagg ttaatcttgt
ctgggggaag acaagcaaac aatttggcag tcaaatcggc gcttatgcga caagttacat ctgttcattt tgtggttatt
ctccatatta tgaatttgaa aattctaagt cgggagatga agaaggggct agagataatc tatatcagat gaagaaattg
agtcgcccct ctcttgaaga tttcctccaa ggaaatccgg tttataagac atttagggat tttgataagt ataaaaacga
tcaacggttg caaaagacgg gtgataaaga tggtgaatgg aaaacacaca gagggaatac tgcaatatac gcctgtcaaa
agtgtagaca tatctctgat gcggatatcc aagcatcata ttggattgct ttgaagcaag ttgtaagaga tttttataaa
gacaaagaga tggatggtga tttgattcaa ggagataata aagacaagag aaaagtaaac gagcttaata gacttattgg
agtacataaa gatgtgccta taataaataa aaatttaata acatcactcg acataaactt actataga
CasY.3 Candidatus vogelbacteria amino acid sequence 1200 aa
(SEQ ID NO: 13):
MKAKKSFYNQKRKFGKRGYRLHDERIAYSGGIGSMRSIKYELKDSYGIAGLRNR
IADATISDNKWLYGNINLNDYLEWRSSKTDKQIEDGDRESSLLGFWLEALRLGFVFSKQSHAP
NDFNETALQDLFETLDDDLKHVLDRKKWCDFIKIGTPKINDQGRLKKQIKNLLKGNKREEIEKTL
NESDDELKEKINRIADVFAKNKSDKYTIFKLDKPNTEKYPRINDVQVAFFCHPDFEEITERDRTK
TLDLIINRFNKRYEITENKKDDKTSNRMALYSLNQGYIPRVLNDLFLFVKDNEDDFSQFLSDLEN
FFSFSNEQIKIIKERLKKLKKYAEPIPGKPQLADKWDDYASDFGGKLESWYSNRIEKLKKIPESV
SDLRNNLEKIRNVLKKQNNASKILELSQKIIEYIRDYGVSFEKPEIIKFSWINKTKDGQKKVFYVA
KMADREFIEKLDLWMADLRSQLNEYNQDNKVSFKKKGKKIEELGVLDFALNKAKKNKSTKNE
NGWQQKLSESIQSAPLFFGEGNRVRNEEVYNLKDLLFSEIKNVENILMSSEAEDLKNIKIEYKE
DGAKKGNYVLNVLARFYARFNEDGYGGWNKVKTVLENIAREAGTDFSKYGNNNNRNAGRFY
LNGRERQVFTLIKFEKSITVEKILELVKLPSLLDEAYRDLVNENKNHKLRDVIQLSKTIMALVLSH
SDKEKQIGGNYIHSKLSGYNALISKRDFISRYSVCITTNGTQCKLAIGKGKSKKGNEIDRYFYAF
QFFKNDDSKINLKVIKNNSHKNIDFNDNENKINALQVYSSNYQIQFLDWFFEKHQGKKTSLEVG
GSFTIAEKSLTIDWSGSNPRVGFKRSDTEEKRVFVSQPFTLIPDDEDKERRKERMIKTKNRFIG
IDIGEYGLAWSLIEVDNGDKNNRGIRQLESGFITDNQQQVLKKNVKSWRQNQIRQTFTSPDTKI
ARLRESLIGSYKNQLESLMVAKKANLSFEYEVSGFEVGGKRVAKIYDSIKRGSVRKKIDNNSQ
NDQSWGKKGINEWSFETTAAGTSQFCTHCKRVVSSLAIVDIEEYELKDYNDNLFKVKINDGEV
RLLGKKGWRSGEKIKGKELFGPVKDAMRPNVDGLGMKIVKRKYLKLDLRDWVSRYGNMAIFI
CPYVDCHHISHADKQAAFNIAVRGYLKSVNPDRAIKHGDKGLSRDFLCQEEGKLNFEQIGLL
CasY.3 Candidatus vogelbacteria nucleic acid sequence
(SEQ ID NO: 14):
atgaaa gctaaaaaaa gtttttataa tcaaaagcgg aagttcggta aaagaggtta tcgtcttcac
gatgaacgta tcgcgtattc aggagggatt ggatcgatgc gatctattaa atatgaattg aaggattcgt atggaattgc
tgggcttcgt aatcgaatcg ctgacgcaac tatttctgat aataagtggc tgtacgggaa tataaatcta aatgattatt
tagagtggcg atcttcaaag actgacaaac agattgaaga cggagaccga gaatcatcac tcctgggttt ttggctggaa
gcgttacgac tgggattcgt gttttcaaaa caatctcatg ctccgaatga ttttaacgag accgctctac aagatttgtt
tgaaactctt gatgatgatt tgaaacatgt tcttgatagg aaaaaatggt gtgactttat caagatagga acacctaaga
caaatgacca aggtcgttta aaaaaacaaa tcaagaattt gttaaaagga aacaagagag aggaaattga aaaaactctc
aatgaatcag acgatgaatt gaaagagaaa ataaacagaa ttgccgatgt ttttgcaaaa aataagtctg ataaatacac
aattttcaaa ttagataaac ccaatacgga aaaatacccc agaatcaacg atgttcaggt ggcgtttttt tgtcatcccg
attttgagga aattacagaa cgagatagaa caaagactct agatctgatc attaatcggt ttaataagag atatgaaatt
accgaaaata aaaaagatga caaaacttca aacaggatgg ccttgtattc cttgaaccag ggctatattc ctcgcgtcct
gaatgattta ttcttgtttg tcaaagacaa tgaggatgat tttagtcagt ttttatctga tttggagaat ttcttctctt tttccaacga
acaaattaaa ataataaagg aaaggttaaa aaaacttaaa aaatatgctg aaccaattcc cggaaagccg caacttgctg
ataaatggga cgattatgct tctgattttg gcggtaaatt ggaaagctgg tactccaatc gaatagagaa attaaagaag
attccggaaa gcgtttccga tctgcggaat aatttggaaa agatacgcaa tgttttaaaa aaacaaaata atgcatctaa
aatcctggag ttatctcaaa agatcattga atacatcaga gattatggag tttcttttga aaagccggag ataattaagt
tcagctggat aaataagacg aaggatggtc agaaaaaagt tttctatgtt gcgaaaatgg cggatagaga attcatagaa
aagcttgatt tatggatggc tgatttacgc agtcaattaa atgaatacaa tcaagataat aaagtttctt tcaaaaagaa
aggtaaaaaa atagaagagc tcggtgtctt ggattttgct cttaataaag cgaaaaaaaa taaaagtaca aaaaatgaaa
atggctggca acaaaaattg tcagaatcta ttcaatctgc cccgttattt tttggcgaag ggaatcgtgt acgaaatgaa
gaagtttata atttgaagga ccttctgttt tcagaaatca agaatgttga aaatatttta atgagctcgg aagcggaaga
cttaaaaaat ataaaaattg aatataaaga agatggcgcg aaaaaaggga actatgtctt gaatgtcttg gctagatttt
acgcgagatt caatgaggat ggctatggtg gttggaacaa agtaaaaacc gttttggaaa atattgcccg agaggcgggg
actgattttt caaaatatgg aaataataac aatagaaatg ccggcagatt ttatctaaac ggccgcgaac gacaagtttt
tactctaatc aagtttgaaa aaagtatcac ggtggaaaaa atacttgaat tggtaaaatt acctagccta cttgatgaag
cgtatagaga tttagtcaac gaaaataaaa atcataaatt acgcgacgta attcaattga gcaagacaat tatggctctg
gttttatctc attctgataa agaaaaacaa attggaggaa attatatcca tagtaaattg agcggataca atgcgcttat
ttcaaagcga gattttatct cgcggtatag cgtgcaaacg accaacggaa ctcaatgtaa attagccata ggaaaaggca
aaagcaaaaa aggtaatgaa attgacaggt atttctacgc ttttcaattt tttaagaatg acgacagcaa aattaattta
aaggtaatca aaaataattc gcataaaaac atcgatttca acgacaatga aaataaaatt aacgcattgc aagtgtattc
atcaaactat cagattcaat tcttagactg gttttttgaa aaacatcaag ggaagaaaac atcgctcgag gtcggcggat
cttttaccat cgccgaaaag agtttgacaa tagactggtc ggggagtaat ccgagagtcg gttttaaaag aagcgacacg
gaagaaaaga gggtttttgt ctcgcaacca tttacattaa taccagacga tgaagacaaa gagcgtcgta aagaaagaat
gataaagacg aaaaaccgtt ttatcggtat cgatatcggt gaatatggtc tggcttggag tctaatcgaa gtggacaatg
gagataaaaa taatagagga attagacaac ttgagagcgg ttttattaca gacaatcagc agcaagtctt aaagaaaaac
gtaaaatcct ggaggcaaaa ccaaattcgt caaacgttta cttcaccaga cacaaaaatt gctcgtcttc gtgaaagttt
gatcggaagt tacaaaaatc aactggaaag tctgatggtt gctaaaaaag caaatcttag ttttgaatac gaagtttccg
ggtttgaagt tgggggaaag agggttgcaa aaatatacga tagtataaag cgtgggtcgg tgcgtaaaaa ggataataac
tcacaaaatg atcaaagttg gggtaaaaag ggaattaatg agtggtcatt cgagacgacg gctgccggaa catcgcaatt
ttgtactcat tgcaagcggt ggagcagttt agcgatagta gatattgaag aatatgaatt aaaagattac aacgataatt
tatttaaggt aaaaattaat gatggtgaag ttcgtctcct tggtaagaaa ggttggagat ccggcgaaaa gatcaaaggg
aaagaattat ttggtcccgt caaagacgca atgcgcccaa atgttgacgg actagggatg aaaattgtaa aaagaaaata
tctaaaactt gatctccgcg attgggtttc aagatatggg aatatggcta ttttcatctg tccttatgtc gattgccacc atatctctca
tgcggataaa caagctgctt ttaatattgc cgtgcgaggg tatttgaaaa gcgttaatcc tgacagagca ataaaacacg
gagataaagg tttgtctagg gactttttgt gccaagaaga gggtaagctt aattttgaac aaatagggtt attatgaa
CasY.4 Candidatus parcubacteria amino acid sequence 1210 aa
(SEQ ID NO: 15):
MSKRHPRISGVKGYRLHAQRLEYTGKSGAMRTIKYPLYSSPSGGRTVPREIVSA
INDDYVGLYGLSNFDDLYNAEKRNEEKVYSVLDFWYDCVQYGAVFSYTAPGLLKNVAEVRGG
SYELTKILKGSHLYDELQIDKVIKFLNKKEISRANGSLDKLKKDIIDCFKAEYRERHKDQCNKLA
DDIKNAKKDAGASLGERQKKLFRDFFGISEQSENDKPSFTNPLNLTCCLLPFDTVNNNRNRGE
VLFNKLKEYAQKLDKNEGSLEMVVEYIGIGNSGTAFSNFLGEGFLGRLRENKITELKKAMMDIT
DAWRGQEQEEELEKRLRILAALTIKLREPKFDNHWGGYRSDINGKLSSWLQNYINQTVKIKED
LKGHKKDLKKAKEMINRFGESDTKEEAVVSSLLESIEKIVPDDSADDEKPDIPAIAIYRRFLSDG
RLTLNRFVQREDVQEALIKERLEAEKKKKPKKRKKKSDAEDEKETIDFKELFPHLAKPLKLVPN
FYGDSKRELYKKYKNAAIYTDALWKAVEKIYKSAFSSSLKNSFFDTDFDKDFFIKRLQKIFSVYR
RFNTDKWKPIVKNSFAPYCDIVSLAENEVLYKPKQSRSRKSAAIDKNRVRLPSTENIAKAGIAL
ARELSVAGFDWKDLLKKEEHEEYIDLIELHKTALALLLAVTETQLDISALDFVENGTVKDFMKTR
DGNLVLEGRFLEMFSQSIVFSELRGLAGLMSRKEFITRSAIQTMNGKQAELLYIPHEFQSAKITT
PKEMSRAFLDLAPAEFATSLEPESLSEKSLLKLKQMRYYPHYFGYELTRTGQGIDGGVAENAL
RLEKSPVKKREIKCKQYKTLGRGQNKIVLYVRSSYYQTQFLEWFLHRPKNVQTDVAVSGSFLI
DEKKVKTRWNYDALTVALEPVSGSERVFVSQPFTIFPEKSAEEEGQRYLGIDIGEYGIAYTALE
ITGDSAKILDQNFISDPQLKTLREEVKGLKLDQRRGTFAMPSTKIARIRESLVHSLRNRIHHLAL
KHKAKIVYELEVSRFEEGKOXIKKVYATLKKADVYSEIDADKNLQTTVWGKLAVASEISASYTS
QFCGACKKLWRAEMQVDETITTQELIGTVRVIKGGTLIDAIKDFMRPPIFDENDTPFPKYRDFC
DKHHISKKMRGNSCLFICPFCRANADADIQASQTIALLRYVKEEKKVEDYFERFRKLKNIKVLG
QMKKI
CasY.4 Candidatus parcubacteria nucleic acid sequence
(SEQ ID NO: 16):
atgagtaagc gacatcctag aattagcggc gtaaaagggt accgtttgca tgcgcaacgg ctggaatata
ccggcaaaag tggggcaatg cgaacgatta aatatcctct ttattcatct ccgagcggtg gaagaacggt tccgcgcgag
atagtttcag caatcaatga tgattatgta gggctgtacg gtttgagtaa ttttgacgat ctgtataatg cggaaaagcg
caacgaagaa aaggtctact cggttttaga tttttggtac gactgcgtcc aatacggcgc ggttttttcg tatacagcgc
cgggtctttt gaaaaatgtt gccgaagttc gcgggggaag ctacgaactt acaaaaacgc ttaaagggag ccatttatat
gatgaattgc aaattgataa agtaattaaa tttttgaata aaaaagaaat ttcgcgagca aacggatcgc ttgataaact
gaagaaagac atcattgatt gcttcaaagc agaatatcgg gaacgacata aagatcaatg caataaactg gctgatgata
ttaaaaatgc aaaaaaagac gcgggagctt ctttagggga gcgtcaaaaa aaattatttc gcgatttttt tggaatttca
gagcagtctg aaaatgataa accgtctttt actaatccgc taaacttaac ctgctgttta ttgccttttg acacagtgaa
taacaacaga aaccgcggcg aagttttgtt taacaagctc aaggaatatg ctcaaaaatt ggataaaaac gaagggtcgc
ttgaaatgtg ggaatatatt ggcatcggga acagcggcac tgccttttct aattttttag gagaagggtt tttgggcaga
ttgcgcgaga ataaaattac agagctgaaa aaagccatga tggatattac agatgcatgg cgtgggcagg aacaggaaga
agagttagaa aaacgtctgc ggatacttgc cgcgcttacc ataaaattgc gcgagccgaa atttgacaac cactggggag
ggtatcgcag tgatataaac ggcaaattat ctagctggct tcagaattac ataaatcaaa cagtcaaaat caaagaggac
ttaaagggac acaaaaagga cctgaaaaaa gcgaaagaga tgataaatag gtttggggaa agcgacacaa
aggaagaggc ggttgtttca tctttgcttg aaagcattga aaaaattgtt cctgatgata gcgctgatga cgagaaaccc
gatattccag ctattgctat ctatcgccgc tttctttcgg atggacgatt aacattgaat cgctttgtcc aaagagaaga
tgtgcaagag gcgctgataa aagaaagatt ggaagcggag aaaaagaaaa aaccgaaaaa gcgaaaaaag
aaaagtgacg ctgaagatga aaaagaaaca attgacttca aggagttatt tcctcatctt gccaaaccat taaaattggt
gccaaacttt tacggcgaca gtaagcgtga gctgtacaag aaatataaga acgccgctat ttatacagat gctctgtgga
aagcagtgga aaaaatatac aaaagcgcgt tctcgtcgtc tctaaaaaat tcattttttg atacagattt tgataaagat ttttttatta
agcggcttca gaaaattttt tcggtttatc gtcggtttaa tacagacaaa tggaaaccga ttgtgaaaaa ctctttcgcg
ccctattgcg acatcgtctc acttgcggag aatgaagttt tgtataaacc gaaacagtcg cgcagtagaa aatctgccgc
gattgataaa aacagagtgc gtctcccttc cactgaaaat atcgcaaaag ctggcattgc cctcgcgcgg gagctttcag
tcgcaggatt tgactggaaa gatttgttaa aaaaagagga gcatgaagaa tacattgatc tcatagaatt gcacaaaacc
gcgcttgcgc ttcttcttgc cgtaacagaa acacagcttg acataagcgc gttggatttt gtagaaaatg ggacggtcaa
ggattttatg aaaacgcggg acggcaatct ggttttggaa gggcgtttcc ttgaaatgtt ctcgcagtca attgtgtttt
cagaattgcg cgggcttgcg ggtttaatga gccgcaagga atttatcact cgctccgcga ttcaaactat gaacggcaaa
caggcggagc ttctctacat tccgcatgaa ttccaatcgg caaaaattac aacgccaaag gaaatgagca gggcgtttct
tgaccttgcg cccgcggaat ttgctacatc gcttgagcca gaatcgcttt cggagaagtc attattgaaa ttgaagcaga
tgcggtacta tccgcattat tttggatatg agcttacgcg aacaggacag gggattgatg gtggagtcgc ggaaaatgcg
ttacgacttg agaagtcgcc agtaaaaaaa cgagagataa aatgcaaaca gtataaaact ttgggacgcg gacaaaataa
aatagtgtta tatgtccgca gttcttatta tcagacgcaa tttttggaat ggtttttgca tcggccgaaa aacgttcaaa
ccgatgttgc ggttagcggt tcgtttctta tcgacgaaaa gaaagtaaaa actcgctgga attatgacgc gcttacagtc
gcgcttgaac cagtttccgg aagcgagcgg gtctttgtct cacagccgtt tactattttt ccggaaaaaa gcgcagagga
agaaggacag aggtatcttg gcatagacat cggcgaatac ggcattgcgt atactgcgct tgagataact ggcgacagtg
caaagattct tgatcaaaat tttatttcag acccccagct taaaactctg cgcgaggagg tcaaaggatt aaaacttgac
caaaggcgcg ggacatttgc catgccaagc acgaaaatcg cccgcatccg cgaaagcctt gtgcatagtt tgcggaaccg
catacatcat cttgcgttaa agcacaaagc aaagattgtg tatgaattgg aagtgtcgcg ttttgaagag ggaaagcaaa
aaattaagaa agtctacgct acgttaaaaa aagcggatgt gtattcagaa attgacgcgg ataaaaattt acaaacgaca
gtatggggaa aattggccgt tgcaagcgaa atcagcgcaa gctatacaag ccagttttgt ggtgcgtgta aaaaattgtg
gcgggcggaa atgcaggttg acgaaacaat tacaacccaa gaactaatcg gcacagttag agtcataaaa gggggcactc
ttattgacgc gataaaggat tttatgcgcc cgccgatttt tgacgaaaat gacactccat ttccaaaata tagagacttt
tgcgacaagc atcacatttc caaaaaaatg cgtggaaaca gctgtttgtt catttgtcca ttctgccgcg caaacgcgga
tgctgatatt caagcaagcc aaacaattgc gcttttaagg tatgttaagg aagagaaaaa ggtagaggac tactttgaac
gatttagaaa gctaaaaaac attaaagtgc tcggacagat gaagaaaata tgatag
CasY.5 Candidatus komeilibacteria amino acid sequence 1192 aa
(SEQ ID NO: 17):
MAESKQMQCRKCGASMKYEVIGLGKKSCRYMCPDCGNHTSARKIQNKKKRDK
KYGSASKAQSQRIAVAGALYPDKKVQTIKTYKYPADLNGEVHDRGVAEKIEQAIQEDEIGLLGP
SSEYACWIASQKCISEPYSVVDFWFDAVCAGGVFAYSGARLLSTVLQLSGEESVLRAALASSP
FVDDINLAQAEKFLAVSRRTGQDKLGKRIGECFAEGRLEALGIKDRMREFVQAIDVAQTAGQR
FAAKLKIFGISQMPEAKQWNNDSGLTVCILPDYYVPEENRADQLVVIIRRLREIAYCMGIEDEA
GFEHLGIDPGALSNFSNGNPKRGFLGRLLNNDIIALANNMSAMTPYWEGRKGELIERLAWLKH
RAEGLYLKEPHFGNSWADHRSRIFSRIAGWLSGCAGKLKIAKDOISGVRTDLELLKRLLDAVP
QSAPSPDFIASISALDRFLEAAESSQDPAEQVRALYAFHLNAPAVRSIANKAVQRSDSQEWLIK
ELDAVDHLEFNKAFPFFSDTGKKKKKGANSNGAPSEEEYTETESIQQPEDAEQEVNGQEGN
GASKNQKKFQRIPRFFGEGSRSEYRILTEAPQYFDMFCNNMRAIFMQLESQPRKAPRDFKCF
LQNRLQKLYKQTFLNARSNKCRALLESVLISWGEFYTYGANEKKFRLRHEASERSSDPDYVV
QQALEIARRLFLFGFEWRDCSAGERVDLVEIHKKAISFLLAITQAEVSVGSYNWLGNSTVSRYL
SVAGTDTLYGTQLEEFLNATVLSQMRGLAIRLSSQELKDGFDVQLESSCQDNLQHLLVYRAS
RDLAACKRATCPAELDPKILVLPAGAFIASVMKMIERGDEPLAGAYLRHRPHSFGWQIRVRGV
AEVGMDQGTALAFQKPTESEPFKIKPFSAQYGPVLWLNSSSYSQSQYLDGFLSQPKNWSMR
VLPQAGSVRVEQRVALIWNLQAGKMRLERSGARAFFMPVPFSFRPSGSGDEAVLAPNRYLG
LFPHSGGIEYAVVDVLDSAGFKILERGTIAVNGFSQKRGERQEEAHREKQRRGISDIGRKKPV
QAEVDAANELHRKYTDVATRLGCRIVVQWAPQPKPGTAPTAQTVYARAVRTEAPRSGNQED
HARMKSSWGYTWSTYWEKRKPEDILGISTQVYWTGGIGESCPAIRATSTQTEWEKEEVVFG
RLKKFFPS
CasY.5 Candidatus komeilibacteria nucleic acid sequence
(SEQ ID NO: 18):
accaaccacc tattgcgtct ttttcgctca ttttagcaaa agtggctgtc tagacataca ggtggaaagg
tgagagtaaa gacatggcct gaatagcgtc ctcgtcctcg tctagacata caggtggaaa ggtgagagta aagaccggag
cactcatcct ctcactctat tttgtctaga catacaggtg gaaaggtgag agtaaagaca aaccgtgcca cactaaaccg
atgagtctag acatacaggt ggaaaggtga gagtaaagac tcaagtaact acctgttctt tcacaagtct agacatacag
gtggaaaggt gagagtaaag actcaagtaa ctacctgttc tttcacaagt ctagacctgc aggtggtaag gtgagagtaa
agactcaagt aactacctgt tctttcacaa gtctagacct gcaggtggta aggtgagagt aaagactttt atcctcctct
ctatgcttct gagtctagac atttaggtgg aaaggtgaga gtaaagactt gtggagatcc atgaacttcg gcagtctaga
cctgcaggtg gaaaggtgag agtaaagacg tccttcacac gatcttcctc tgttagtcta ggcctgcagg tggaaaggtg
agagtaaaga cgcataagcg taattgaagc tctctccggt ccagaccttg tcgcgcttgt gttgcgacaa aggcggagtc
cgcaataagt tctttttaca atgttttttc cataaaaccg atacaatcaa gtatcggttt tgcttttttt atgaaaatat gttatgctat
gtgctcaaat aaaaatatca ataaaatagc gtttttttga taatttatcg ctaaaattat acataatcac gcaacattgc
cattctcaca caggagaaaa gtcatggcag aaagcaagca gatgcaatgc cgcaagtgcg gcgcaagcat gaagtatgaa
gtaattggat tgggcaagaa gtcatgcaga tatatgtgcc cagattgcgg caatcacacc agcgcgcgca agattcagaa
caagaaaaag cgcgacaaaa agtatggatc cgcaagcaaa gcgcagagcc agaggatagc tgtggctggc gcgctttatc
cagacaaaaa agtgcagacc ataaagacct acaaataccc agcggatctg aatggcgaag ttcatgacag aggcgtcgca
gagaagattg agcaggcgat tcaggaagat gagatcggcc tgcttggccc gtccagcgaa tacgcttgct ggattgcttc
acaaaaacaa agcgagccgt attcagttgt agatttttgg tttgacgcgg tgtgcgcagg cggagtattc gcgtattctg
gcgcgcgcct gctttccaca gtcctccagt tgagtggcga ggaaagcgtt ttgcgcgctg ctttagcatc tagcccgttt
gtagatgaca ttaatttggc gcaagcggaa aagttcctag ccgttagccg gcgcacaggc caagataagc taggcaagcg
cattggagaa tgtttcgcgg aaggccggct tgaagcgctt ggcatcaaag atcgcatgcg cgaattcgtg caagcgattg
atgtggccca aaccgcgggc cagcggttcg cggccaagct aaagatattc ggcatcagtc agatgcctga agccaagcaa
tggaacaatg attccgggct cactgtatgt attttgccgg attattatgt cccggaagaa aaccgcgcgg accagctggt
tgttttgctt cggcgcttac gcgagatcgc gtattgcatg ggaattgagg atgaagcagg atttgagcat ctaggcattg
accctggcgc tctttccaat ttttccaatg gcaatccaaa gcgaggattt ctcggccgcc tgctcaataa tgacattata
gcgctggcaa acaacatgtc agccatgacg ccgtattggg aaggcagaaa aggcgagttg attgagcgcc ttgcatggct
taaacatcgc gctgaaggat tgtatttgaa agagccacat ttcggcaact cctgggcaga ccaccgcagc aggattttca
gtcgcattgc gggctggctt tccggatgcg cgggcaagct caagattgcc aaggatcaga tttcaggcgt gcgtacggat
ttgtttctgc tcaagcgcct tctggatgcg gtaccgcaaa gcgcgccgtc gccggacttt attgcttcca tcagcgcgct
ggatcggttt ttggaagcgg cagaaagcag ccaggatccg gcagaacagg tacgcgcttt gtacgcgttt catctgaacg
cgcctgcggt ccgatccatc gccaacaagg cggtacagag gtctgattcc caggagtggc ttatcaagga actggatgct
gtagatcacc ttgaattcaa caaagcattt ccgttttttt cggatacagg aaagaaaaag aagaaaggag cgaatagcaa
cggagcgcct tctgaagaag aatacacgga aacagaatcc attcaacaac cagaagatgc agagcaggaa gtgaatggtc
aagaaggaaa tggcgcttca aagaaccaga aaaagtttca gcgcattcct cgatttttcg gggaagggtc aaggagtgag
tatcgaattt taacagaagc gccgcaatat tttgacatgt tctgcaataa tatgcgcgcg atctttatgc agctagagag
tcagccgcgc aaggcgcctc gtgatttcaa atgctttctg cagaatcgtt tgcagaagct ttacaagcaa acctttctca
atgctcgcag taataaatgc cgcgcgcttc tggaatccgt ccttatttca tggggagaat tttatactta tggcgcgaat
gaaaagaagt ttcgtctgcg ccatgaagcg agcgagcgca gctcggatcc ggactatgtg gttcagcagg cattggaaat
cgcgcgccgg cttttcttgt tcggatttga gtggcgcgat tgctctgctg gagagcgcgt ggatttggtt gaaatccaca
aaaaagcaat ctcatttttg cttgcaatca ctcaggccga ggtttcagtt ggttcctata actggcttgg gaatagcacc
gtgagccggt atctttcggt tgctggcaca gacacattgt acggcactca actggaggag tttttgaacg ccacagtgct
ttcacagatg cgtgggctgg cgattcggct ttcatctcag gagttaaaag acggatttga tgttcagttg gagagttcgt
gccaggacaa tctccagcat ctgctggtgt atcgcgcttc gcgcgacttg gctgcgtgca aacgcgctac atgcccggct
gaattggatc cgaaaattct tgttctgccg gctggtgcgt ttatcgcgag cgtaatgaaa atgattgagc gtggcgatga
accattagca ggcgcgtatt tgcgtcatcg gccgcattca ttcggctggc agatacgggt tcgtggagtg gcggaagtag
gcatggatca gggcacagcg ctagcattcc agaagccgac tgaatcagag ccgtttaaaa taaagccgtt ttccgctcaa
tacggcccag tactttggct taattcttca tcctatagcc agagccagta tctggatgga tttttaagcc agccaaagaa
ttggtctatg cgggtgctac ctcaagccgg atcagtgcgc gtggaacagc gcgttgctct gatatggaat ttgcaggcag
gcaagatgcg gctggagcgc tctggagcgc gcgcgttttt catgccagtg ccattcagct tcaggccgtc tggttcagga
gatgaagcag tattggcgcc gaatcggtac ttgggacttt ttccgcattc cggaggaata gaatacgcgg tggtggatgt
attagattcc gcgggtttca aaattcttga gcgcggtacg attgcggtaa atggcttttc ccagaagcgc ggcgaacgcc
aagaggaggc acacagagaa aaacagagac gcggaatttc tgatataggc cgcaagaagc cggtgcaagc
tgaagttgac gcagccaatg aattgcaccg caaatacacc gatgttgcca ctcgtttagg gtgcagaatt gtggttcagt
gggcgcccca gccaaagccg ggcacagcgc cgaccgcgca aacagtatac gcgcgcgcag tgcggaccga
agcgccgcga tctggaaatc aagaggatca tgctcgtatg aaatcctctt ggggatatac ctggagcacc tattgggaga
agcgcaaacc agaggatatt ttgggcatct caacccaagt atactggacc ggcggtatag gcgagtcatg tcccgcagtc
gcggttgcgc ttttggggca cattagggca acatccactc aaactgaatg ggaaaaagag gaggttgtat tcggtcgact
gaagaagttc tttccaagct agacgatctt tttaaaaact gggctgctgg ctatcgtatg gtcagtagct cttatttttt tacttgatat
atggtattat
CasY.6 Candidatus kerfeldbacteria amino acid sequence 1287 aa
(SEQ ID NO: 19):
MKRILNSLKVAALRLLFRGKGSELVKTVKYPLVSPVQGAVEELAEAIRHDNLHLFGQKEIVDLM
EKDEGTQVYSVVDFWLDTLRLGMFFSPSANALKITLGKFNSDQVSPFRKVLEQSPFFLAGRLK
VEPAERILSVEIRKIGKRENRVENYAADVETCFIGQLSSDEKQSIQKLANDIWDSKDHEEQRML
KADFFAIPLIKDPKAVTEEDPENETAGKQKPLELCVCLVPELYTRGFGSIADFLVQRLTLLRDK
MSTDTAEDCLEYVGIEEEKGNGMNSLLGTFLKNLQGDGFEQIFQFMLGSYVGWQGKEDVLR
ERLDLLAEKVKRLPKPKFAGEWSGHRMFLHGQLKSWSSNFFRLFNETRELLESIKSDIQHATM
LISYVEEKGGYHPQLLSQYRKLMEQLPALRTKVLDPEIEMTHMSEAVRSYIMIHKSVAGFLPDL
LESLDRDKDREFLLSIFPRIPKIDKKTKEIVAWELPGEPEEGYLFTANNLFRNFLENPKHVPRFM
AERIPEDWTRLRSAPVWFDGMVKQWQKVVNQLVESPGALYQFNESFLRQRLQAMLTVYKR
DLQTEKFLKLLADVCRPLVDFFGLGGNDIIFKSCQDPRKQVVQTVIPLSVPADVYTACEGLAIR
LRETLGFEWKNLKGHEREDFLRLHQLLGNLLFWIRDAKLVVKLEDWMNNPCVQEYVEARKAI
DLPLEIFGFEVPIFLNGYLFSELRQLELLLRRKSVMTSYSVKTTGSPNRLFQLVYLPLNPSDPEK
KNSNNFQERLDTPTGLSRRFLDLTLDAFAGKLLTDPVTQELKTMAGFYDHLFGFKLPCKLAAM
SNHPGSSSKMVVLAKPKKGVASNIGFEPIPDPAHPVFRVRSSWPELKYLEGLLYLPEDTPLTIE
LAETSVSCQSVSSVAFDLKNLTTILGRVGEFRVTADQPFKLTPIIPEKEESFIGKTYLGLDAGER
SGVGFAIVTVDGDGYEVQRLGVHEDTQLMALQQVASKSLKEPVFQPLRKGTFRQQERIRKSL
RGCYWNFYHALMIKYRAKVVHEESVGSSGLVGQWLRAFQKDLKKADVLPKKGGKNGVDKKK
RESSAQDTLWGGAFSKKEEQQIAFEVQAAGSSQFCLKCGVVWFQLGMREVNRVQESGVVLD
WNRSIVTFLIESSGEKVYGFSPQQLEKGFRPDIETFKKMVRDFMRPPMFDRKGRPAAAYERF
VLGRRHRRYRFDKVFEERFGRSALFICPRVGCGNFDHSSEQSAVVLALIGYIADKEGMSGKKL
VYVRLAELMAEWKLKKLERSRVEEQSSAQ
CasY.6 Candidatus kerfeldbacteria nucleic acid sequence
(SEQ ID NO: 20):
atgaagag aattctgaac agtctgaaag ttgctgcctt gagacttctg tttcgaggca aaggttctga
attagtgaag acagtcaaat atccattggt ttccccggtt caaggcgcgg ttgaagaact tgctgaagca attcggcacg
acaacctgca cctttttggg cagaaggaaa tagtggatct tatggagaaa gacgaaggaa cccaggtgta ttcggttgtg
gatttttggt tggataccct gcgtttaggg atgtttttct caccatcagc gaatgcgttg aaaatcacgc tgggaaaatt
caattctgat caggtttcac cttttcgtaa ggttttggag cagtcacctt tttttcttgc gggtcgcttg aaggttgaac ctgcggaaag
gatactttct gttgaaatca gaaagattgg taaaagagaa aacagagttg agaactatgc cgccgatgtg gagacatgct
tcattggtca gctttcttca gatgagaaac agagtatcca gaagctggca aatgatatct gggatagcaa ggatcatgag
gaacagagaa tgttgaaggc ggattttttt gctatacctc ttataaaaga ccccaaagct gtcacagaag aagatcctga
aaatgaaacg gcgggaaaac agaaaccgct tgaattatgt gtttgtcttg ttcctgagtt gtatacccga ggtttcggct
ccattgctga ttttctggtt cagcgactta ccttgctgcg tgacaaaatg agtaccgaca cggcggaaga ttgcctcgag
tatgttggca ttgaggaaga aaaaggcaat ggaatgaatt ccttgctcgg cacttttttg aagaacctgc agggtgatgg
ttttgaacag atttttcagt ttatgcttgg gtcttatgtt ggctggcagg ggaaggaaga tgtactgcgc gaacgattgg
atttgctggc cgaaaaagtc aaaagattac caaagccaaa atttgccgga gaatggagtg gtcatcgtat gtttctccat
ggtcagctga aaagctggtc gtcgaatttc ttccgtcttt ttaatgagac gcgggaactt ctggaaagta tcaagagtga
tattcaacat gccaccatgc tcattagcta tgtggaagag aaaggaggct atcatccaca gctgttgagt cagtatcgga
agttaatgga acaattaccg gcgttgcgga ctaaggtttt ggatcctgag attgagatga cgcatatgtc cgaggctgtt
cgaagttaca ttatgataca caagtctgta gcgggatttc tgccggattt actcgagtct ttggatcgag ataaggatag
ggaatttttg ctttccatct ttcctcgtat tccaaagata gataagaaga cgaaagagat cgttgcatgg gagctaccgg
gcgagccaga ggaaggctat ttgttcacag caaacaacct tttccggaat tttcttgaga atccgaaaca tgtgccacga
tttatggcag agaggattcc cgaggattgg acgcgtttgc gctcggcccc tgtgtggttt gatgggatgg tgaagcaatg
gcagaaggtg gtgaatcagt tggttgaatc tccaggcgcc ctttatcagt tcaatgaaag ttttttgcgt caaagactgc
aagcaatgct tacggtctat aagcgggatc tccagactga gaagtttctg aagctgctgg ctgatgtctg tcgtccactc
gttgattttt tcggacttgg aggaaatgat attatcttca agtcatgtca ggatccaaga aagcaatggc agactgttat
tccactcagt gtcccagcgg atgtttatac agcatgtgaa ggcttggcta ttcgtctccg cgaaactctt ggattcgaat
ggaaaaatct gaaaggacac gagcgggaag attttttacg gctgcatcag ttgctgggaa atctgctgtt ctggatcagg
gatgcgaaac ttgtcgtgaa gctggaagac tggatgaaca atccttgtgt tcaggagtat gtggaagcac gaaaagccat
tgatcttccc ttggagattt tcggatttga ggtgccgatt tttctcaatg gctatctctt ttcggaactg cgccagctgg aattgttgct
gaggcgtaag tcggtgatga cgtcttacag cgtcaaaacg acaggctcgc caaataggct cttccagttg gtttacctac
ctctaaaccc ttcagatccg gaaaagaaaa attccaacaa ctttcaggag cgcctcgata cacctaccgg tttgtcgcgt
cgttttctgg atcttacgct ggatgcattt gctggcaaac tcttgacgga tccggtaact caggaactga agacgatggc
cggtttttac gatcatctct ttggcttcaa gttgccgtgt aaactggcgg cgatgagtaa ccatccagga tcctcttcca
aaatggtggt tctggcaaaa ccaaagaagg gtgttgctag taacatcggc tttgaaccta ttcccgatcc tgctcatcct
gtgttccggg tgagaagttc ctggccggag ttgaagtacc tggaggggtt gttgtatctt cccgaagata caccactgac
cattgaactg gcggaaacgt cggtcagttg tcagtctgtg agttcagtcg ctttcgattt gaagaatctg acgactatct
tgggtcgtgt tggtgaattc agggtgacgg cagatcaacc tttcaagctg acgcccatta ttcctgagaa agaggaatcc
ttcatcggga agacctacct cggtcttgat gctggagagc gatctggcgt tggtttcgcg attgtgacgg ttgacggcga
tgggtatgag gtgcagaggt tgggtgtgca tgaagatact cagcttatgg cgcttcagca agtcgccagc aagtctctta
aggagccggt tttccagcca ctccgtaagg gcacatttcg tcagcaggag cgcattcgca aaagcctccg cggttgctac
tggaatttct atcatgcatt gatgatcaag taccgagcta aagttgtgca tgaggaatcg gtgggttcat ccggtctggt
ggggcagtgg ctgcgtgcat ttcagaagga tctcaaaaag gctgatgttc tgcccaagaa gggtggaaaa aatggtgtag
acaaaaaaaa gagagaaagc agcgctcagg ataccttatg gggaggagct ttctcgaaga aggaagagca
gcagatagcc tttgaggttc aggcagctgg atcaagccag ttttgtctga agtgtggttg gtggtttcag ttggggatgc
gggaagtaaa tcgtgtgcag gagagtggcg tggtgctgga ctggaaccgg tccattgtaa ccttcctcat cgaatcctca
ggagaaaagg tatatggttt cagtcctcag caactggaaa aaggctttcg tcctgacatc gaaacgttca aaaaaatggt
aagggatttt atgagacccc ccatgtttga tcgcaaaggt cggccggccg cggcgtatga aagattcgta ctgggacgtc
gtcaccgtcg ttatcgcttt gataaagttt ttgaagagag atttggtcgc agtgctcttt tcatctgccc gcgggtcggg
tgtgggaatt tcgatcactc cagtgagcag tcagccgttg tccttgccct tattggttac attgctgata aggaagggat
gagtggtaag aagcttgttt atgtgaggct ggctgaactt atggctgagt ggaagctgaa gaaactggag agatcaaggg
tggaagaaca gagctcggca caataa

Any of the gene editor effectors herein can also be tagged with Tev or any other suitable homing protein domains. According to Wolfs, et al. (Proc Natl Acad Sci USA. 2016 Dec. 27; 113(52):14988-14993. doi: 10.1073/pnas.1616343114. Epub 2016 Dec. 12), Tev is an RNA-guided dual active site nuclease that generates two noncompatible DNA breaks at a target site, effectively deleting the majority of the target site such that it cannot be regenerated.

The siRNA and C2c2 in the compositions herein are targeted to a particular gene in a virus or gene mRNA. The siRNA can have a first strand of a duplex substantially identical to the nucleotide sequence of a portion of the viral gene or gene mRNA sequence. The second strand of the siRNA duplex is complementary to both the first strand of the siRNA duplex and to the same portion of the viral gene mRNA. Isolated siRNA can include short double-stranded RNA from about 17 nucleotides to about 29 nucleotides in length, preferably from about 19 to about 25 nucleotides in length, that are targeted to the target mRNA. The siRNA's comprise a sense RNA strand and a complementary antisense RNA strand annealed together by standard Watson-Crick base-pairing interactions. The sense strand comprises a nucleic acid sequence which is substantially identical to a target sequence contained within the target mRNA. The siRNA of the invention can be obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference. Preferably, the siRNA of the invention is chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Alternatively, siRNA can also be expressed from recombinant circular or linear DNA plasmids using any suitable promoter. Suitable promoters for expressing siRNA of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the siRNA in a particular tissue or in a particular intracellular environment. The siRNA expressed from recombinant plasmids can either be isolated from cultured cell expression systems by standard techniques or can be expressed intracellularly. siRNA of the invention can be expressed from a recombinant plasmid either as two separate, complementary RNA molecules, or as a single RNA molecule with two complementary regions. For example, siRNA can be useful in targeting JC Virus, BKV, or SV40 polyomaviruses (U.S. Patent Application Publication No. 2007/0249552 to Khalili, et al.), wherein siRNA is used which targets JCV agnoprotein gene or large T antigen gene mRNA and wherein the sense RNA strand comprises a nucleotide sequence substantially identical to a target sequence of about 19 to about 25 contiguous nucleotides in agnoprotein gene or large T antigen gene mRNA.

Various viruses can be targeted by the compositions and methods of the present invention. Depending on whether they are lytic or lysogenic, different compositions and methods can be used as appropriate.

TABLE 2 lists viruses in the picornaviridae/hepeviridae/flaviviridae families and their method of replication.

TABLE 2

| | | |
|---|---|---|
| [symbol] patitis A | [symbol] RNA viral genome | [symbol] ic/Lysogenic Replication cycle |
| [symbol] patitis B | [symbol] NA-RT viral genome | [symbol] ogenic Replication cycle |
| [symbol] patitis C | [symbol] RNA viral genome | [symbol] ic Replication cycle |
| [symbol] patitis D | [symbol] RNA viral genome | [symbol] ic/Lysogenic Replication cycle |
| [symbol] patitis E | [symbol] RNA viral genome | |
| [symbol] xsachievirus | | [symbol] ic Replication cycle |

[symbol]

indicates data missing or illegible when filed

It should be noted that Hepatitis D propagates only in the presence of Hepatitis B, therefore, the composition particularly useful in treating Hepatitis D is one that targets Hepatitis B as well, such as two or more CRISPR-associated nucleases such as Cas9, Cpf1, C2c1, C2c3, TevCas9, Archaea Cas9, CasY.1-CasY.6, and CasX gRNAs, Argonaute endonuclease gDNAs and other gene editors to treat the lysogenic virus and siRNAs/miRNAs/shRNAs/RNAi to treat the lytic virus.

TABLE 3 lists viruses in the herpesviridae family and their method of replication.

TABLE 3

| | | |
|---|---|---|
| HSV-1 (HHV1) | dsDNA viral genome | [symbol] tic/Lysogenic Replication cycle |
| HSV-2 (HHV2) | dsDNA viral genome | [symbol] tic/Lysogenic Replication cycle |
| Cytomegalovirus (HHV5) | dsDNA viral genome | [symbol] tic/Lysogenic Replication cycle |
| Epstein-Barr Virus (HHV4) | dsDNA viral genome | [symbol] tic/Lysogenic Replication cycle |
| Varicella Zoster Virus (HHV3) | dsDNA viral genome | [symbol] tic/Lysogenic Replication cycle |
| Roseolovirus (HHV6A/B) | | |
| HHV7 | | |
| HHV8 | | |

[symbol]

indicates data missing or illegible when filed

TABLE 4 lists viruses in the orthomyxoviridae family and their method of replication.

TABLE 4

| | |
|---|---|
| Influenza Types A, B, C, D | −ssRNA viral genome |

TABLE 5 lists viruses in the retroviridae family and their method of replication.

TABLE 5

| | | |
|---|---|---|
| HIV1 and HIV2 | +ssRNA viral genome | Lytic/Lysogenic Replication cycle |
| HTLV1 and HTLV2 | +ssRNA viral genome | Lytic/Lysogenic Replication cycle |
| Rous Sarcoma Virus | +ssRNA viral genome | Lytic/Lysogenic Replication cycle |

TABLE 6 lists viruses in the papillomaviridae family and their method of replication.

TABLE 6

| | | |
|---|---|---|
| [symbol] V family | DNA viral genome | [symbol] dding from desquamating cells (semi-lysogenic) |

[symbol]

indicates data missing or illegible when filed

TABLE 7 lists viruses in the flaviviridae family and their method of replication.

TABLE 7

| | | |
|---|---|---|
| Yellow Fever | +ssRNA viral genome | Budding/Lysogenic Replication |
| Zika | +ssRNA viral genome | Budding/Lysogenic Replication |
| Dengue | +ssRNA viral genome | Budding/Lysogenic Replication |
| West Nile | +ssRNA viral genome | Budding/Lysogenic Replication |
| Japanese Encephalitis | +ssRNA viral genome | Budding/Lysogenic Replication |

TABLE 8 lists viruses in the reoviridae family and their method of replication.

TABLE 8

| | | |
|---|---|---|
| Rota | dsRNA viral genome | Lytic Replication cycle |
| Seadornvirus | dsRNA viral genome | Lytic Replication cycle |
| Coltivirus | dsRNA viral genome | Lytic Replication cycle |

TABLE 9 lists viruses in the rhabdoviridae family and their method of replication.

TABLE 9

| | | |
|---|---|---|
| Lyssa Virus (Rabies) | −ssRNA viral genome | Budding/Lysogenic Replication |
| Vesiculovirus | −ssRNA viral genome | Budding/Lysogenic Replication |
| Cytorhabdovirus | −ssRNA viral genome | Budding/Lysogenic Replication |

TABLE 10 lists viruses in the bunyanviridae family and their method of replication.

TABLE 10

| | | |
|---|---|---|
| Hantaan Virus | tripartite −ssRNA viral genome | Budding/Lysogenic Replication |
| Rift Valley Fever | tripartite −ssRNA viral genome | Budding/Lysogenic Replication |
| Bunyamwera Virus | tripartite −ssRNA viral genome | Budding/Lysogenic Replication |

TABLE 11 lists viruses in the arenaviridae family and their method of replication.

TABLE 11

| | | |
|---|---|---|
| Lassa Virus | ssRNA viral genome | Budding/Lysogenic Replication |
| Junin Virus | ssRNA viral genome | Budding/Lysogenic Replication |
| Machupo Virus | ssRNA viral genome | Budding/Lysogenic Replication |
| Sabia Virus | ssRNA viral genome | Budding/Lysogenic Replication |
| Taca ibe Virus | ssRNA viral genome | Budding/Lysogenic Replication |
| Flexal Virus | ssRNA viral genome | Budding/Lysogenic Replication |
| Whitewater Arroyo Virus | ssRNA viral genome | Budding/Lysogenic Replication |

TABLE 12 lists viruses in the filoviridae family and their method of replication.

TABLE 12

| | | |
|---|---|---|
| Ebola | RNA viral genome | Budding/Lysogenic Replication |
| Marburg Virus | RNA viral genome | Budding/Lysogenic Replication |

TABLE 13 lists viruses in the polyomaviridae family and their method of replication.

TABLE 13

| | | |
|---|---|---|
| JC Virus | dsDNA circular viral genome | [symbol] ytic/Lysogenic Replication cycl [symbol] |
| BK Virus | dsDNA circular viral genome | [symbol] ytic/Lysogenic Replication cycl [symbol] |

[symbol]

indicates data missing or illegible when filed

The compositions of the present invention can be used to treat either active or latent viruses. The compositions of the present invention can be used to treat individuals in which latent virus is present but the individual has not yet presented symptoms of the virus. The compositions can target virus in any cells in the individual, such as, but not limited to, CD4+ lymphocytes, macrophages, fibroblasts, monocytes, T lymphocytes, B lymphocytes, natural killer cells, dendritic cells such as Langerhans cells and follicular dendritic cells, hematopoietic stem cells, endothelial cells, brain microglial cells, and gastrointestinal epithelial cells.

In the present invention, when any of the compositions are contained within an expression vector, the CRISPR endonuclease can be encoded by the same nucleic acid or vector as the gRNA sequences. Alternatively or in addition, the CRISPR endonuclease can be encoded in a physically separate nucleic acid from the gRNA sequences or in a separate vector.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). As noted above, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col El, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Yeast expression systems can also be used. For example, the non-fusion pYES2 vector (XbaI, SphI, ShoI, NotI, GstXI, EcoRI, BstXI, BamH1, SacI, KpnI, and HindIII cloning sites; Invitrogen) or the fusion pYESHisA, B, C (XbaI, SphI, ShoI, NotI, BstXI, EcoRI, BamH1, SacI, KpnI, and HindIII cloning sites, N-terminal peptide purified with ProBond resin and cleaved with enterokinase; Invitrogen), to mention just two, can be employed according to the invention. A yeast two-hybrid expression system can also be prepared in accordance with the invention.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Suitable nucleic acid delivery systems include recombinant viral vector, typically sequence from at least one of an adenovirus, adenovirus-associated virus (AAV), helper-dependent adenovirus, retrovirus, or hemagglutinating virus of Japan-liposome (HVJ) complex. In such cases, the viral vector comprises a strong eukaryotic promoter operably linked to the polynucleotide e.g., a cytomegalovirus (CMV) promoter. The recombinant viral vector can include one or more of the polynucleotides therein, preferably about one polynucleotide. In some embodiments, the viral vector used in the invention methods has a pfu (plague forming units) of from about 10⁸ to about 5×10¹⁰ pfu. In embodiments in which the polynucleotide is to be administered with a non-viral vector, use of between from about 0.1 nanograms to about 4000 micrograms will often be useful e.g., about 1 nanogram to about 100 micrograms.

Additional vectors include viral vectors, fusion proteins and chemical conjugates. Retroviral vectors include Moloney murine leukemia viruses and HIV-based viruses. One HIV-based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

Pox viral vectors introduce the gene into the cells cytoplasm. Avipox virus vectors result in only a short-term expression of the nucleic acid. Adenovirus vectors, adeno-associated virus vectors and herpes simplex virus (HSV) vectors may be an indication for some invention embodiments. The adenovirus vector results in a shorter-term expression (e.g., less than about a month) than adeno-associated virus, in some embodiments, may exhibit much longer expression. The particular vector chosen will depend upon the target cell and the condition being treated. The selection of appropriate promoters can readily be accomplished. An example of a suitable promoter is the 763-base-pair cytomegalovirus (CMV) promoter. Other suitable promoters which may be used for gene expression include, but are not limited to, the Rous sarcoma virus (RSV) (Davis, et al., Hum Gene Ther 4:151 (1993)), the SV40 early promoter region, the herpes thymidine kinase promoter, the regulatory sequences of the metallothionein (MMT) gene, prokaryotic expression vectors such as the β-lactamase promoter, the tac promoter, promoter elements from yeast or other fungi such as the GAL4 promoter, the ADH (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells, insulin gene control region which is active in pancreatic beta cells, immunoglobulin gene control region which is active in lymphoid cells, mouse mammary tumor virus control region which is active in testicular, breast, lymphoid and mast cells, albumin gene control region which is active in liver, alpha-fetoprotein gene control region which is active in liver, alpha 1-antitrypsin gene control region which is active in the liver, beta-globin gene control region which is active in myeloid cells, myelin basic protein gene control region which is active in oligodendrocyte cells in the brain, myosin light chain-2 gene control region which is active in skeletal muscle, and gonadotropic releasing hormone gene control region which is active in the hypothalamus. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely affect the metabolism of the organism being treated. The cassette can also be bound to a nucleic acid binding moiety in a synthetic delivery system, such as the system disclosed in WO 95/22618.

If desired, the polynucleotides of the invention can also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Feigner and Holm, Bethesda Res. Lab. Focus, 11(2):21 (1989) and Maurer, R.A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the expressed products intracellularly. See for example, Chen et al, BioTechniques, 34: 167-171 (2003), which is incorporated herein, by reference, in its entirety.

As described above, the compositions of the present invention can be prepared in a variety of ways known to one of ordinary skill in the art. Regardless of their original source or the manner in which they are obtained, the compositions of the invention can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment, e.g., the treatment of a subject having a virus or at risk for contracting a virus. When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intra-arterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This invention also includes pharmaceutical compositions which contain, as the active ingredient, nucleic acids and vectors described herein in combination with one or more pharmaceutically acceptable carriers. The terms “pharmaceutically acceptable” (or “pharmacologically acceptable”) refer to molecular entities and compositions that do not produce an adverse, allergic or other untoward reaction when administered to an animal or a human, as appropriate. The methods and compositions disclosed herein can be applied to a wide range of species, e.g., humans, non-human primates (e.g., monkeys), horses or other livestock, dogs, cats, ferrets or other mammals kept as pets, rats, mice, or other laboratory animals. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of HIV infection, for example, brain macrophages, microglia, astrocytes, and gut-associated lymphoid cells. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding a CRISPR-associated endonuclease and a guide RNA is operatively linked to a promoter or enhancer-promoter combination. Promoters and enhancers are described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol-modified (PEGylated) low molecular weight LPEI.

The nucleic acids and vectors may also be applied to a surface of a device (e.g., a catheter) or contained within a pump, patch, or other drug delivery device. The nucleic acids and vectors of the invention can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In any of the methods described herein, treatment can be in vivo (directly administering the composition) or ex vivo (for example, a cell or plurality of cells, or a tissue explant, can be removed from a subject having an viral infection and placed in culture, and then treated with the composition). Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems. In any of the methods described herein, the amount of the compositions administered is enough to inactivate all of the virus present in the individual. An individual is effectively treated whenever a clinically beneficial result ensues. This may mean, for example, a complete resolution of the symptoms of a disease, a decrease in the severity of the symptoms of the disease, or a slowing of the disease's progression. The present methods may also include a monitoring step to help optimize dosing and scheduling as well as predict outcome.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion overtime. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. The term “effective” as used herein refers to any amount that induces a desired response while not inducing significant toxicity in the patient. Such an amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method of excising undesired DNA or RNA from cells, including the steps of:
administering a composition including a vector encoding at least one gene editor and at least one gRNA to an individual; and
excising the undesired DNA or RNA from cells, wherein cut repair is made by microhomology-mediated end joining (MMEJ).

2. The method of claim 1, wherein the at least one gene editor targets DNA and is chosen from the group consisting of Argonaute proteins, C2c1, C2c2, C2c3, Cas9, Cpf1, TevCas9, Archaea Cas9, CasY.1, CasY.2, CasY.3, CasY.4, CasY.5, CasY.6, CasX, and combinations thereof.

3. The method of claim 1, wherein the composition further includes a gene editor that targets viral RNA chosen from the group consisting of C2c2 and RNase P RNA.

4. The method of claim 3, wherein the composition further includes a composition that targets viral RNA chosen from the group consisting of siRNA, miRNA, shRNAs, and RNAi.

5. The method of claim 1, wherein said excising step includes removing a replication critical segment of the viral DNA or RNA.

6. The method of claim 1, wherein said excising step is further defined as excising an entire viral genome of a virus from a host cell.

7. The method of claim 1, wherein the undesired DNA or RNA is a lysogenic virus chosen from the group consisting of hepatitis A, hepatitis B, hepatitis D, HSV-1, HSV-2, cytomegalovirus, Epstein-Barr virus, Varicella Zoster virus, HIV1, HIV2, HTLV1, HTLV2, Rous Sarcoma virus, HPV virus, yellow fever, zika, dengue, West Nile, Japanese encephalitis, lyssa virus, vesiculovirus, cytohabdovirus, Hantaan virus, Rift Valley virus, Bunyamwera virus, Lassa virus, Junin virus, Machupo virus, Sabia virus, Tacaribe virus, Flexal virus, Whitewater Arroyo virus, ebola, Marburg virus, JC virus, and BK virus.

8. The method of claim 1, wherein the undesired DNA or RNA is a lytic virus chosen from the group consisting of hepatitis A, hepatitis C, hepatitis D, coxsachievirus, HSV-1, HSV-2, cytomegalovirus, Epstein-Barr virus, varicella zoster virus, HIV1, HIV2, HTLV1, HTLV2, Rous Sarcoma virus, rota, seadornvirus, coltivirus, JC virus, and BK virus.

9. The method of claim 1, wherein the undesired DNA or RNA is cancer chosen from the group consisting of adenoid cystic carcinoma, adrenal gland tumors, amyloidosis, anal cancer, appendix cancer, astrocytoma, ataxia-telangiectasia, attenuated familial adenomatous polyposis, Beckwith-Wiedermann Syndrome, bile duct cancer, Birt-Hogg-Dube Syndrome, bladder cancer, bone cancer, brain stem glioma, brain tumors, breast cancer, carcinoid tumors, Carney complex, central nervous system tumors, cervical cancer, colorectal cancer, Cowden syndrome, craniopharyngioma, desmoplastic infantile ganglioglioma, endocrine tumors, ependymoma, esophageal cancer, Ewing sarcoma, eye cancer, eyelid cancer, fallopian tube cancer, familial adenomatous polyposis, familial malignant melanoma, familial non-VHL clear cell renal cell carcinoma, gallbladder cancer, Gardner Syndrome, gastrointestinal stromal tumor, germ cell tumor, gestational trophoblastic disease, head and neck cancer, diffuse gastric cancer, leiomyomatosis and renal cell cancer, mixed polyposis syndrome, pancreatitis, papillary renal cell carcinoma, HIV and AIDS-related cancer, islet cell tumors, juvenile polyposis syndrome, kidney cancer, lacrimal gland tumor, laryngeal and hypopharyngeal cancer, acute lymphoblastic leukemia, acute lymphocytic leukemia, acute myeloid leukemia, B-cell prolymphocytic leukemia, hairy cell leukemia, chronic lymphocytic leukemia, chronic myeloid leukemia, chronic T-cell lymphocytic leukemia, eosinophilic leukemia, Li-Fraumeni Syndrome, liver cancer, lung cancer, Hodgkin lymphoma, Non-Hodgkin lymphoma, Lynch Syndrome, mastocytosis, medulloblastoma, melanoma, meningioma, mesothelioma, Muir-Torre Syndrome, multiple endocrine neoplasia type 1, multiple endocrine neoplasia type 2, multiple myeloma, myelodysplastic syndromes, MYH-associated polyposis, nasal cavity and paranasal sinus cancer, nasopharyngeal cancer, neuroblastoma, neuroendocrine tumors, neurofibromatosis type 1, neurofibromatosis type 2, nevoid basal cell carcinoma syndrome, oral and oropharyngeal cancer, osteosarcoma, ovarian cancer, pancreatic cancer, parathyroid cancer, penile cancer, Peutz-Jeghers Syndrome, pituitary gland tumors, pleuropulmonary blastoma, prostate cancer, retinoblastoma, rhabdomyosarcoma, salivary gland cancer, sarcoma, alveolar soft part and cardiac sarcoma, Kaposi sarcoma, skin cancer, small bowel cancer, stomach cancer, testicular cancer, thymoma, thyroid cancer, tuberous sclerosis syndrome, Turcot Syndrome, unknown primary, uterine cancer, vaginal cancer, Von Hippel-Lindau Syndrome, Wilms tumors, and Xeroderma pigmentosum.
